SEQUENCE LISTING (30 sequences)

SEQ ID NO: 1 - protein, 424 aa, Artificial Sequence
  Chemically synthesized PEPAa amino acid sequence Fig. 1
MQLLQSLIVAVCFSYGVLSLPHGPSNQHKARSFKVERVRRGTGALHGPAALRKAYRKYGIAPSSFNIDLADFKPITTTHAAAGSEIAEPDQTGAVSATSVENDAEFVSPVLIGGQKIVMTFDTGSSDFWVFDTNLNETLTGHTEYNPSNSSTFKKMDGYTFDVSYGDDSYASGPVGTDTVNIGGAIVKEQAFGVPDQVSQSFIEDTNSNGLVGLGFSSINTIKPEAQDTFFANVAPSLDEPVMTASLKADGVGEYEFGTIDKDKYQGNIANISVDSSNGYWQFSTPKYSVADGELKDIGSLNTSIADTGTSLMLLDEDVVTAYYAQVPNSVYVSSAGGYIYPCNTTLPSFSLVLGESSLATIPGNLINFSKVGTNTTTGQALCFGGIQSNGNTSLQILGDIFLKAFFVVFDMRGPSLGVASPKN

SEQ ID NO: 2 - protein, 426 aa, Artificial Sequence
  Chemically synthesized PEPAb amino acid sequence Fig. 2
MATKIKLIPNLNYKRSGTKSYVHLMRKYRFHPTKPGPYTLSSSIQQTGRPYTEKPIGGRAHIRQLVRKKSTTSDEVGEVPAEDVQNDSMYLATVGIGTPAQNLKLDFDTGSADLWVWSNKLPSTLLSENKTHAIFDSSKSSTFKTLEGESWQISYGDGSSASGSVGTDDVNIGGVVVKNQAVELAEKMSSTFAQGEGDGLLGLAFSNINTVQPKSVKTPVENMILQDDIPKSAELFTAKLDTWRDTDDESFYTFGFIDQDLVKTAGEEVYYTPVDNSQGFWLFNSTSATVNGKTINRSGNTAIADTGTTLALVDDDTCEAIYSAIDGAYYDQEVQGWIYPTDTAQDKLPTVSFAVGEKQFVVQKEDLAFSEAKTGYVYGGIQSRGDMTMDILGDTFLKSIYAIFDVGNLRFGAVQREELRQSLKSE

SEQ ID NO: 3 - protein, 453 aa, Artificial Sequence
  Chemically synthesized PEPAc amino acid sequence Fig. 3
MYIPVGTLATASLLAGAALAAPTPSPLKGRNIVRRSGSHTVYKPAAFAAPSHNKASSKYLELSKTKSKGNVNPRSAAYVKRSTSSGSSSLISLFEGEEFATSITIGGDSFDVIVDTGSSDTWVVKTGFTCIDLDTGRETSESSCDFGSTWTVESSFKEIEGEEFAIEYGDGEYLYGVMGNETVALADITVDQTIGVVTEAAWEGDGTTSGLTGLAYPALTSAYSTTTDEQIVYSNIITTMWEEGLIEPLFSLAIERDVSGAAGYLALGGLPPVDFVEDFTKTSILVTNIEGYSKAYDFYTINIDAVTLNGKSLTSAGGDDIQYIMQVDSGTTLNYYPTSIAEEINAAFSPAATYSDEEGAYIVDCDATPPTHGITISGKTFYINPLDMILDAGTDDEGNTICISGIVDGGSDTSEDLYILGDTFQKNVVTVFDIGATELRFAARENYTSNDTY

SEQ ID NO: 4 - protein, 480 aa, Artificial Sequence
  Chemically synthesized PEPAd amino acid sequence Fig. 4
MHLPQRLVTAACLCASATAFIPYTIKLDTSDDISARDSLARRFLPVPNPSDALADDSTSSASDESLSLNIKRIPVRRDNDFKIVVAETPSWSNTAALDQDGSDISYISVVNIGSDEKSMYMLLDTGGSDTWVFGSNCTSTPCTMHNTFGSDDSSTLEMTSEEWSVGYGTGSVSGLLGKDKLTIANVTVRMTFGLASNASDNFESYPMDGILGLGRTNDSSYDNPTFMDAVAESNVFKSNIVGFALSRSPAKDGTVSFGTTDKDKYTGDITYTDTVGSDSYWRIPVDDVYVGGTSCDFSNKSAIIDTGTSYAMLPSSDSKTLHSLIPGAKSSGSYHIIPCNTTTKLQVAFSGVNYTISPKDYVGATSGSGCVSNIISYDLFGDDIWLLGDTFLKNVYAVFDYDELRVGFAERSSNTTSASNSTSSGTSSTSGSTTTGSSTTTTSSASSSSSSDAESGSSMTIPAPQYFFSALAIASFMLWL

SEQ ID NO: 5 - DNA, 29 bp, Artificial Sequence
  Synthetic primer
cactcgaggccaccatgcagctcctccag

SEQ ID NO: 6 - DNA, 27 bp, Artificial Sequence
  Synthetic primer
aggaaactagttcttgggagaggcaac

SEQ ID NO: 7 - DNA, 1410 bp, Artificial Sequence
  Chemically synthesized pepAa amplicon Fig. 5
cactcgaggccaccatgcagctcctccagtccctcattgttgccgtttgcttcagctacggcgtcctctccttaccccatggcccgtcaaaccagcacaaagcacgttccttcaaggttgaacgggtccgtcgtggaaccggtgctctgcatgggcccgctgctctccgcaaagcataccggaagtacggaatagctcccagcagtttcaacatcgatctggcagactttaaacccattacgacaacccatgctgctgctgggagcgagattgcagagcctgatcagactggcgctgtcagtgctacttccgtcgagaacgatgccgagttcgtttcgcctgttcttattggcggccagaagatcgtcatgacatttgacactggttcttctgacttgtaagtcttggatgcagctgtttactctttggtacagtgattaacgtcgatctacagttgggtgttcgatacgaatctcaatgaaaccttgacgggacacacggagtacaacccttcgaactcctcgaccttcaagaagatggacggatacaccttcgatgtctcgtatggtgacgactcgtacgcctctggccccgtcggaacggataccgtcaacattggcggcgccattgtcaaggagcaagccttcggtgtccccgaccaggtatcccagtcgttcatcgaggacacgaactccaacggcctggtcgggttgggcttttcctccatcaacaccatcaaaccggaggcgcaagacacgttcttcgccaatgtcgcaccaagtctggacgagcccgtcatgaccgcctcgctcaaggctgacggagtgggcgagtacgagttcggcacgatcgacaaagacaagtaccagggcaacattgccaacatcagcgtggactcatcgaacggatactggcagttctccactcccaagtactccgtggcagacggagagctgaaggacattggaagcttgaacacctcgatcgcggacaccggtacctcccttatgctgctggatgaagacgtggttactgcctactatgcgcaagttcccaactcggtctacgtgagcagtgccggtggttacatctacccctgcaacaccactcttcccagcttctcgcttgtcctcggcgagtcgagcctggccacgatccccggtaacctgatcaatttctccaaggttggcaccaacaccaccaccggacaggcctgtaagttgctccccttcttttgcatgattgaacatgattgactgattgtgctggttagtgtgctttggcggcattcaatccaacggaaacacctcgctgcagattctgggcgatattttcctgaaggcctttttcgttgtcttcgacatgcgcggcccctcgcttggtgttgcctctcccaagaactagtttcct

SEQ ID NO: 8 - DNA, 27 bp, Artificial Sequence
  Synthetic primer
actctagatcaatcatgttcaatcatg

SEQ ID NO: 9 - DNA, 1264 bp, Artificial Sequence
  Chemically synthesized pepAa* (truncated pepAa) amplicon Fig. 9
cactcgaggccaccatgcagctcctccagtccctcattgttgccgtttgcttcagctacggcgtcctctccttaccccatggcccgtcaaaccagcacaaagcacgttccttcaaggttgaacgggtccgtcgtggaaccggtgctctgcatgggcccgctgctctccgcaaagcataccggaagtacggaatagctcccagcagtttcaacatcgatctggcagactttaaacccattacgacaacccatgctgctgctgggagcgagattgcagagcctgatcagactggcgctgtcagtgctacttccgtcgagaacgatgccgagttcgtttcgcctgttcttattggcggccagaagatcgtcatgacatttgacactggttcttctgacttgtaagtcttggatgcagctgtttactctttggtacagtgattaacgtcgatctacagttgggtgttcgatacgaatctcaatgaaaccttgacgggacacacggagtacaacccttcgaactcctcgaccttcaagaagatggacggatacaccttcgatgtctcgtatggtgacgactcgtacgcctctggccccgtcggaacggataccgtcaacattggcggcgccattgtcaaggagcaagccttcggtgtccccgaccaggtatcccagtcgttcatcgaggacacgaactccaacggcctggtcgggttgggcttttcctccatcaacaccatcaaaccggaggcgcaagacacgttcttcgccaatgtcgcaccaagtctggacgagcccgtcatgaccgcctcgctcaaggctgacggagtgggcgagtacgagttcggcacgatcgacaaagacaagtaccagggcaacattgccaacatcagcgtggactcatcgaacggatactggcagttctccactcccaagtactccgtggcagacggagagctgaaggacattggaagcttgaacacctcgatcgcggacaccggtacctcccttatgctgctggatgaagacgtggttactgcctactatgcgcaagttcccaactcggtctacgtgagcagtgccggtggttacatctacccctgcaacaccactcttcccagcttctcgcttgtcctcggcgagtcgagcctggccacgatccccggtaacctgatcaatttctccaaggttggcaccaacaccaccaccggacaggcctgtaagttgctccccttcttttgcatgattgaacatgattgatctagagt

SEQ ID NO: 10 - DNA, 29 bp, Artificial Sequence
  Synthetic primer
aactcgagtccatcatggctaccaaaatc

SEQ ID NO: 11 - DNA, 27 bp, Artificial Sequence
  Synthetic primer
cctctagactactccgacttcaggctc

SEQ ID NO: 12 - DNA, 1303 bp, Artificial Sequence
  Chemically synthesized pepAb amplicon Fig. 10
aactcgagtccatcatggctaccaaaatcaagctcatccccaatctcaactacaagcgctcaggcaccaagtcctacgtgcacttgatgcgcaagtaccgcttccatcccaccaagcctggtccctacactctcagcagctccatccaacagaccggtcgtccgtacactgaaaagcccatcgggggtcgggcccatatccggcagctggtgcggaagaagagcaccaccagcgatgaggttggcgaggttccggccgaagatgtgcagaacgactccatgtatctggcgaccgtggggatcggaaccccggcgcagaacctgaagttggactttgacactggttcagctgatctttgggtctggtccaacaaactcccctcaacccttctatccgagaacaagacccatgcgatcttcgactcgtccaaatcgagcaccttcaagaccttggaaggtgaatcctggcaaatctcctacggagatggatcctccgcatcagggagtgtgggcaccgacgacgtcaacattggcggcgtagtcgtcaagaaccaagccgttgagctggcagagaagatgtccagcacattcgcccaaggcgaaggggacggattgctcggtctagcattcagcaacatcaacacggtacagccaaagtccgtgaaaacgcccgtcgagaacatgatcctgcaggatgacattcccaagtcggctgagctgttcacggccaagctggatacctggcgggacactgatgacgagtcgttttacacctttggcttcattgaccaggatctggtgaagacggcaggtgaagaggtctactacacccctgtcgataacagtcaaggcttctggctattcaactcgacctccgcgacggtaaatggaaagaccattaaccggtcgggtaacaccgccattgctgataccggtacgacgctggccttggtggacgatgacacgtgtgaggccatttatagtgcaattgacggcgcctattatgatcaggaagtacagggctggatctatccgaccgatacggcgcaggataagctacccactgtgtcgtttgccgtgggtgaaaagcagttcgtggtgcagaaggaggacctggcgttttcggaggcgaagacgggctatgtctatggaggaatccaaagtcgtggtgatatgaccatggacatcttgggagacacatttttgaagagtatttatgctatctttgatgtcgggaacctgcgctttggagccgtccagcgcgaggagttgcgccagagcctgaagtcggagtagtctagagg

SEQ ID NO: 13 - DNA, 30 bp, Artificial Sequence
  Synthetic primer
cactcgagtgccgccatgtatatccccgtc

SEQ ID NO: 14 - DNA, 27 bp, Artificial Sequence
  Synthetic primer
ttgctcagttcgaggtacttggaggag

SEQ ID NO: 15 - DNA, 26 bp, Artificial Sequence
  Synthetic primer
agtacctcgaactgagcaagaccaag

SEQ ID NO: 16 - DNA, 26 bp, Artificial Sequence
  Synthetic primer
tctagtaggtgtcgttggaggtgtag

SEQ ID NO: 17 - DNA, 1471 bp, Artificial Sequence
  Chemically synthesized pepAc amplicon Fig. 13
cactcgagtgccgccatgtatatccccgtcggtacccttgccactgcctcgctccttgccggggctgctctggctgcacccactccctccccgctcaagggaagaaatattgtccgcagaagcggcagccacaccgtctacaagcccgccgccttcgctgctcccagtcacaacaaggcctcctccaagtacctcgaactgagcaagaccaagtccaagggaaatgtgaaccctcgtagtgctgcctacgtgaagcgctccaccagcagtggcagctccagcttgatctccctcttcgaaggcgaggagttcgctacctcaatcaccatcggtggtgattcttttgacgttatcgtcgacaccggttccagtgatacctgggtcgttaagacgggcttcacatgtattgacctcgacaccggccgcgaaacctccgagtcgagctgcgactttggctccacctggactgtcgagagctctttcaaggaaattgagggcgaagaattcgccatcgagtatggtgatggtgagtacctctacggagtgatgggtaacgaaaccgtcgctcttgccgatatcactgtggatcaaaccatcggagtggtcactgaggccgcctgggagggcgacggaaccacctctggcctgactggtcttgcgtaccctgccctgtatgtattctgccccaattactgatagtcaggcttctaacatgtatcagtacgagcgcctactccacaaccaccgacgagcagattgtctacagcaatatcattaccaccatgtgggaggagggcctgatcgaacctctgtttagtctggccattgagcgcgatgtttccggtgccgctggctatctggctctcggcggtctgcctcctgtggacttcgtggaagacttcaccaacacctcaatcctggtcaccaacatcgaaggctactccaaggcctacgacttctacaccatcaacattgacgctgtcactctgaacggcaagagcttgaccagcgctggtggcgacgacatccagtacatcgtacgttgactccagcacccctttacctgatggtgtctaacagatgcaggtcgattcgggcaccaccctgaactactacccaacttccatcgctgaggagatcaacgccgctttctcccctgcggcgacttactcggacgaggagggcgcctacattgtagactgcgacgccacccctcccactcacggcatcaccatcagcggaaagaccttctacatcaaccccctcgacatgatcctcgatgctggcaccgacgacgagggcaacaccatctgcatttccggtatcgttgacggtggtagcgacacctccgaagatctctacattctgggcgacaccttccagaagaatgtcgtcactgtgttcgacattggcgccacggagctgagattcgctgctcgcgagaactacacctccaacgacacctactaga

SEQ ID NO: 18 - DNA, 28 bp, Artificial Sequence
  Synthetic primer
tgactcgagcaagttatgcatctcccac

SEQ ID NO: 19 - DNA, 44 bp, Artificial Sequence
  Synthetic primer
ttctagagccaaagcatgaaggaagcacgctctgcaaatccgac

SEQ ID NO: 20 - DNA, 1274 bp, Artificial Sequence
  Chemically synthesized pepAd* amplicon - Fig.16
tgactcgagcaagttatgcatctcccacagcgtctcgttacagcagcgtgtctttgcgccagtgccacggctttcatcccatacaccatcaaactcgatacgtcggacgacatctcagcccgtgattcattagctcgtcgtttcctgccagtaccaaaaccaagcgatgctctagcagacgattccacctcatctgccagcgatgagtccctgtcactgaacatcaaaaggattcccgttcgtcgtgacaatgatttcaagattgtggtagcggaaactccctcttggtctaacaccgccgctctcgatcaagatggtagcgacatttcatacatctctgtcgtcaacattgggtctgatgagaaatctatgtacatgttgctcgacacaggcggctctgatacctgggttttcggttccaactgcacgtccacaccctgcacgatgcacaataccttcggttcggacgattcttcgacccttgaaatgacatcggaagagtggagtgtgggctatggaactgggtctgtcagcggcttgctaggaaaagacaagctcacgattgcaaatgtcactgtacgcatgactttcggacttgcttccaacgcatcggataacttcgagtcgtacccaatggacggcattctcggtctcggtcgaaccaacgatagttcctacgacaacccaacattcatggatgccgttgcagaaagtaacgttttcaagtcgaatatcgttggcttcgccctttcacgtagccccgccaaggatggcacggtcagctttggcactactgacaaggacaagtacaccggcgatatcacctacaccgataccgtcggatcggacagctattggcgcattcccgtggacgatgtctatgttggcggcacttcatgcgatttctccaacaaatcagccatcatcgataccggaacttcttatgctatgctgccttcaagcgactcgaagacgctgcacagtctcattcccggcgccaaatcttcggggagctaccacattattccgtgcaacacaactactaagctacaagtggcattctctggtgtgaattacaccatctcgccgaaggactacgtgggagcaacttcaggttctggatgcgtttcgaacattatcagctacgacttatttggtgatgacatctggctcctgggtgacacgtttctcaaaaatgtgtatgctgtgtttgactacgatgagttacgggtcggatttgcagagcgtgcttccttcatgctttggctctagaa

SEQ ID NO: 21 - protein, 418 aa, Artificial Sequence
  Chemically synthesized truncated PEPAd* amino acid sequence Fig. 17
MHLPQRLVTAACLCASATAFIPYTIKLDTSDDISARDSLARRFLPVPNPSDALADDSTSSASDESLSLNIKRIPVRRDNDFKIVVAETPSWSNTAALDQDGSDISYISVVNIGSDEKSMYMLLDTGGSDTWVFGSNCTSTPCTMHNTFGSDDSSTLEMTSEEWSVGYGTGSVSGLLGKDKLTIANVTVRMTFGLASNASDNFESYPMDGILGLGRTNDSSYDNPTFMDAVAESNVFKSNIVGFALSRSPAKDGTVSFGTTDKDKYTGDITYTDTVGSDSYWRIPVDDVYVGGTSCDFSNKSAIIDTGTSYAMLPSSDSKTLHSLIPGAKSSGSYHIIPCNTTTKLQVAFSGVNYTISPKDYVGATSGSGCVSNIISYDLFGDDIWLLGDTFLKNVYAVFDYDELRVGFAERASFMLWL

SEQ ID NO: 22 - protein, 62 aa, Artificial Sequence
  Chemically synthesized C-terminal GPI anchor sequence of PEPAd deleted in PEPAd* - Fig. 17
SSNTTSASNSTSSGTSSTSGSTTTGSSTTTTSSASSSSSSDAESGSSMTIPAPQYFFSALAI

SEQ ID NO: 23 - protein, 394 aa, Artificial Sequence
  Chemically synthesized PEPAa* amino acid sequence - Fig. 24
MQLLQSLIVAVCFSYGVLSLPHGPSNQHKARSFKVERVRRGTGALHGPAALRKAYRKYGIAPSSFNIDLADFKPITTTHAAAGSEIAEPDQTGAVSATSVENDAEFVSPVLIGGQKIVMTFDTGSSDFWVFDTNLNETLTGHTEYNPSNSSTFKKMDGYTFDVSYGDDSYASGPVGTDTVNIGGAIVKEQAFGVPDQVSQSFIEDTNSNGLVGLGFSSINTIKPEAQDTFFANVAPSLDEPVMTASLKADGVGEYEFGTIDKDKYQGNIANISVDSSNGYWQFSTPKYSVADGELKDIGSLNTSIADTGTSLMLLDEDVVTAYYAQVPNSVYVSSAGGYIYPCNTTLPSFSLVLGESSLATIPGNLINFSKVGTNTTTGQACKLLPFFCMIEHD

SEQ ID NO: 24 - DNA, 25 bp, Artificial Sequence
  Synthetic primer
aactagagccaaagcatgaaggaag

SEQ ID NO: 25 - DNA, 1460 bp, Artificial Sequence
  Chemically synthesized pepAd amplicon - Fig. 20
tgactcgagcaagttatgcatctcccacagcgtctcgttacagcagcgtgtctttgcgccagtgccacggctttcatcccatacaccatcaaactcgatacgtcggacgacatctcagcccgtgattcattagctcgtcgtttcctgccagtaccaaaaccaagcgatgctctagcagacgattccacctcatctgccagcgatgagtccctgtcactgaacatcaaaaggattcccgttcgtcgtgacaatgatttcaagattgtggtagcggaaactccctcttggtctaacaccgccgctctcgatcaagatggtagcgacatttcatacatctctgtcgtcaacattgggtctgatgagaaatctatgtacatgttgctcgacacaggcggctctgatacctgggttttcggttccaactgcacgtccacaccctgcacgatgcacaataccttcggttcggacgattcttcgacccttgaaatgacatcggaagagtggagtgtgggctatggaactgggtctgtcagcggcttgctaggaaaagacaagctcacgattgcaaatgtcactgtacgcatgactttcggacttgcttccaacgcatcggataacttcgagtcgtacccaatggacggcattctcggtctcggtcgaaccaacgatagttcctacgacaacccaacattcatggatgccgttgcagaaagtaacgttttcaagtcgaatatcgttggcttcgccctttcacgtagccccgccaaggatggcacggtcagctttggcactactgacaaggacaagtacaccggcgatatcacctacaccgataccgtcggatcggacagctattggcgcattcccgtggacgatgtctatgttggcggcacttcatgcgatttctccaacaaatcagccatcatcgataccggaacttcttatgctatgctgccttcaagcgactcgaagacgctgcacagtctcattcccggcgccaaatcttcggggagctaccacattattccgtgcaacacaactactaagctacaagtggcattctctggtgtgaattacaccatctcgccgaaggactacgtgggagcaacttcaggttctggatgcgtttcgaacattatcagctacgacttatttggtgatgacatctggctcctgggtgacacgtttctcaaaaatgtgtatgctgtgtttgactacgatgagttacgggtcggatttgcagagcgttcctcgaacaccacctctgcgtcgaactctacgagctctggaacaagcagcacctcgggttccactacaacgggcagctcaacgactacgacgagctctgctagctctagtagttcatctgatgctgaatcaggaagtagcatgaccattcccgctcctcagtatttcttctctgctctggcgattgcttccttcatgctttggctctagtt

SEQ ID NO: 26 - DNA, 28 bp, Artificial Sequence
  Synthetic primer
atgctacttgattcagcatcagatgaac

SEQ ID NO: 27 - DNA, 1458 bp, Artificial Sequence
  Synthetic PEPAd** amplicon - Fig. 25
tgactcgagcaagttatgcatctcccacagcgtctcgttacagcagcgtgtctttgcgccagtgccacggctttcatcccatacaccatcaaactcgatacgtcggacgacatctcagcccgtgattcattagctcgtcgtttcctgccagtaccaaaaccaagcgatgctctagcagacgattccacctcatctgccagcgatgagtccctgtcactgaacatcaaaaggattcccgttcgtcgtgacaatgatttcaagattgtggtagcggaaactccctcttggtctaacaccgccgctctcgatcaagatggtagcgacatttcatacatctctgtcgtcaacattgggtctgatgagaaatctatgtacatgttgctcgacacaggcggctctgatacctgggttttcggttccaactgcacgtccacaccctgcacgatgcacaataccttcggttcggacgattcttcgacccttgaaatgacatcggaagagtggagtgtgggctatggaactgggtctgtcagcggcttgctaggaaaagacaagctcacgattgcaaatgtcactgtacgcatgactttcggacttgcttccaacgcatcggataacttcgagtcgtacccaatggacggcattctcggtctcggtcgaaccaacgatagttcctacgacaacccaacattcatggatgccgttgcagaaagtaacgttttcaagtcgaatatcgttggcttcgccctttcacgtagccccgccaaggatggcacggtcagctttggcactactgacaaggacaagtacaccggcgatatcacctacaccgataccgtcggatcggacagctattggcgcattcccgtggacgatgtctatgttggcggcacttcatgcgatttctccaacaaatcagccatcatcgataccggaacttcttatgctatgctgccttcaagcgactcgaagacgctgcacagtctcattcccggcgccaaatcttcggggagctaccacattattccgtgcaacacaactactaagctacaagtggcattctctggtgtgaattacaccatctcgccgaaggactacgtgggagcaacttcaggttctggatgcgtttcgaacattatcagctacgacttatttggtgatgacatctggctcctgggtgacacgtttctcaaaaatgtgtatgctgtgtttgactacgatgagttacgggtcggatttgcagagcgttcctcgaacaccacctctgcgtcgaactctacgagctctggaacaagcagcacctcgggttccactacaacgggcagctcaacgactacgacgagctctgctagctctagtagttcatctgatgctgaatcaagtagcatgaccattcccgctcctcagtatttcttctctgctctggcgattgcttccttcatgctttggctctagtta

SEQ ID NO: 28 - protein, 479 aa, Artificial Sequence
  Synthetic PEPAd** amino acid sequence - Fig. 26
MHLPQRLVTAACLCASATAFIPYTIKLDTSDDISARDSLARRFLPVPNPSDALADDSTSSASDESLSLNIKRIPVRRDNDFKIVVAETPSWSNTAALDQDGSDISYISVVNIGSDEKSMYMLLDTGGSDTWVFGSNCTSTPCTMHNTFGSDDSSTLEMTSEEWSVGYGTGSVSGLLGKDKLTIANVTVRMTFGLASNASDNFESYPMDGILGLGRTNDSSYDNPTFMDAVAESNVFKSNIVGFALSRSPAKDGTVSFGTTDKDKYTGDITYTDTVGSDSYWRIPVDDVYVGGTSCDFSNKSAIIDTGTSYAMLPSSDSKTLHSLIPGAKSSGSYHIIPCNTTTKLQVAFSGVNYTISPKDYVGATSGSGCVSNIISYDLFGDDIWLLGDTFLKNVYAVFDYDELRVGFAERSSNTTSASNSTSSGTSSTSGSTTTGSSTTTTSSASSSSSSDAESSSMTIPAPQYFFSALAIASFMLWL

SEQ ID NO: 29 - DNA, 27 bp, Artificial Sequence
  Synthetic primer
tgctgaatcaagtagcatgaccattcc

SEQ ID NO: 30 - DNA, 25 bp, Artificial Sequence
  Synthetic primer
taactagagccaaagcatgaaggaa

(19) United States
(12) Patent Application Publication
(10) Pub. No.: US 2011/0294191 A1
(43) Pub. Date: Dec. 1, 2011

(54) INCREASED PRODUCTION OF ASPARTIC PROTEASES IN FILAMENTOUS FUNGAL CELLS

(75) Inventor: Huaming Wang, Fremont, CA (US)

(73) Assignee: DANISCO US INC., Palo Alto, CA (US)

(21) Appl. No.: 13/054,753

(22) PCT Filed: Jul. 28, 2009
(86) PCT No.: PCT/US09/51910

§ 371 Date: Aug. 15, 2011

Related U.S. Application Data
(60) Provisional application No. 61/084,491, filed on Jul. 29, 2008.

(51) Int. Cl. C12N 9/48; C12N 15/80; C07H 21/00; C12N 1/15

(52) U.S. Cl. 435/212; 435/254.11; 435/254.3; 435/254.9; 435/254.6; 435/254.8; 435/320.1; 536/23.2

(57) ABSTRACT

Described are compositions and methods relating to filamentous fungal cells genetically engineered to provide increased production of aspartic proteases, such as PEPAa, PEPAb, PEPAc, and PEPAd. Also described are nucleic acids and methods for making the engineered filamentous fungal cells.

### PRIORITY

The present application claims priority to U.S. Provisional Patent Application Ser. No. 61/084,491, filed on Jul. 29, 2008, which is herein incorporated by reference in its entirety.

### TECHNICAL FIELD

The compositions and methods relate to filamentous fungal cells genetically engineered to provide increased production aspartic protease enzymes, including the PEPA homologs: PEPAa, PEPAb, PEPAc and PEPAd.

### BACKGROUND

Genetic engineering has allowed improvements in microorganisms used as industrial bioreactors, cell factories and in food fermentations. Important enzymes and proteins produced by engineered microorganisms include glucoamylases, α-amylases, cellulases, neutral proteases, and alkaline (or serine) proteases, hormones and antibodies.

For example, enzymes having granular starch hydrolyzing (GSH) activity, such as glucoamylase, are important industrial enzymes used for producing compounds such as organic acids (e.g., lactic acids), amino acids (e.g., glutamic acids), sugar sweetener products (e.g., glucose and high fructose corn syrup), alcohols (e.g., ethanol) and other compounds from starch substrates derived from grains and cereals. In addition, during microbial fermentations, and particularly during simultaneous saccharification and fermentation (SSF), enzymes are needed to reduce the amount of residual starch in the fermentation when granular starch substrates are used as a carbon feed.

Filamentous fungi (e.g., Aspergillus and Trichoderma species) and certain bacteria (e.g., Bacillus species) have been engineered to produce and secrete a large number of useful proteins and metabolites (see, e.g., Bio/Technol. 5:369-76, 713-19, and 1301-04 (1987) and Zukowski, “Production of commercially valuable products,” In: Doi and McGlouglin (eds.) Biology of Bacilli: Applications to Industry, Butterworth-Heinemann, Stoneham. Mass, pp. 311-37 (1992)).

U.S. Pat. No. 7,332,319, which is hereby incorporated by reference, relates to an engineered filamentous fungal host cell comprising a heterologous polynucleotide that encodes an acid-stable alpha amylase (asAA) having GSH activity. This host cell system may be used in combination with a glucoamylase to enhance starch hydrolysis and alcohol production.

However, the occurrence of protein degradation can interfere with efficient production of heterologous proteins in genetically engineered cells. Thus, filamentous fungi have been engineered with reduced or inactivated production of certain proteases.

WO 97/22705, which is hereby incorporated by reference, relates to fungi, which do not produce certain proteases, and can be used as hosts for the production of proteins susceptible to proteolytic degradation by the proteases usually produced.

U.S. Pat. Nos. 5,840,570 and 6,509,171, each of which is hereby incorporated by reference, relate to mutant filamentous fungi which are deficient in a gene for an aspartic protease and are useful hosts for the production of heterologous polypeptides such as chymosin.

U.S. Patent Publication No. 2006/0246545, which is hereby incorporated by reference, relates to recombinant filamentous fungal cells engineered for heterologous protein (e.g., laccase) production by inactivation of chromosomal genes including the aspartic protease genes, pepAa, pepAb, pepAc, and pepAd.

Aspartic proteases are pepsin-like enzymes that are members of the A1 family of peptidases (see, e.g., Rawlings et al., “MEROPS: the peptidase database,” Nucleic Acids Res. 32:160-164 (2004)). Generally, this enzyme family comprises proteins with a three-dimensional structure close to that of pepsin. The three-dimensional structure has two domains with different amino acid sequences, but basically similar folds. The catalytic site is formed at the junction of the two domains and contains two aspartic acid residues, Asp32 and Asp215 (based on human pepsin numbering), one in each domain (see, e.g., Blundell et al., “The aspartic proteinases: an historical overview,” Adv. Exp. Med. Biol. 436:1-13 (1998)). In accordance with the accepted mechanism of the pepsin-like enzyme function (see, e.g., James, “Catalytic pathway of aspartic peptidases,” In: Handbook of Proteolytic Enzymes, Barrett, A. J., Rawlings, N. D., Woessner, J. F. (eds.), Elsevier, London, pp. 12-19 (2004)), the Asp215 is charged and Asp32 has to be protonated for catalysis. The catalytic center exhibits activity in the acidic pH range. Aspartic proteases have been identified from Botrystis cinerea (see, e.g., ten Have et al., “An aspartic proteinase gene family in the filamentous fungus Botrytis cinerea contains members with novel features,” Microbiology 150, 2475-89 (2004)) as well as A. oryzae (see, e.g., Machida et al., “Genome sequencing and analysis of Aspergillus oryzae,” Nature 438:1157-61 (2005)).

At least eight predicted ORFs for aspartic proteases (i.e., members of the A1 family of peptidases) have been identified in the genome sequences of the NRRL3, ATCC 1015, and CBS 513.88 strains of A. niger (see, e.g., Wang et al., “Isolation of four pepsin-like protease genes from Aspergillus niger and analysis of the effect of disruptions on heterologous laccase expression,” Fungal Genetics and Biology 45:17-27 (2008); Pel et al., “Genome sequencing and analysis of the versatile cell factory Aspergillus niger CBS 513.88,” Nat. Biotechnol. 25:221-31 (2007)). One of these eight, PEPA, is known to contribute significantly to acidic proteolysis in A. niger. Deletion of the pepA gene has been found to increase heterologous bovine prochymosin production by more than 66% (see, e.g., Berka et al., “Molecular cloning and deletion of the gene encoding aspergillopepsin A from A. awamori,” Gene 86:153-62 (1990)). It has been shown that the PEPA aspartic protease encoded by pepA in A. niger (also referred to as “aspergillopepsin A”) constitutes ˜80% of the extracellular acidic proteolytic activity (using BSA as a broad substrate to assay proteolytic activity) (see, e.g., van den Hombergh et al., “Disruption of three acid proteases in Aspergillus niger—effects on protease spectrum, intracellular proteolysis, and degradation of target proteins,” Eur. J. Biochem. 247:605-13 (1997)). It has also been shown that a defect in the pepA gene reduced degradation of overexpressed thaumatin in A. niger (see, e.g., Moralejo et al., “Overexpression and lack of degradation of thaumatin in an aspergillopepsin A-defective mutant of Aspergillus awamori containing an insertion in the pepA gene,” Appl. Microbiol. Biotechnol. 54:772-77 (2000)).

The other seven aspartic protease ORFs identified in the various Aspergillus genomes are homologs of the pepA gene. Four of these pepA homolog genes are pepAa, pepAb, pepAc and pepAd, and were reported as encoding proteins of 424, 426, 453 and 480 amino acids, respectively (see, e.g., Wang et al., Fungal Genetics and Biology 45:17-27 (2008)). The inactivation of pepAa, pepAb, or pepAd, in an A. niger strain with inactivated pepA, was found to increase the strain's secretion level of heterologous laccase about 18.7%, 37.0%, and 5.20%, respectively (see, e.g., Wang et al. Fungal Genetics and Biology 45:17-27 (2008)).

Although their inactivation has been shown to improve heterologous protein production, the aspartic proteases are acidic proteases that are useful industrial enzymes in applications such as ethanol production and corn steeping for animal feed production. The ability to produce large amounts of individual aspartic proteases, such as PEPAa, PEPAb, PEPAc, and PEPAd, in filamentous fungal cell systems could provide for these industrial applications.

### SUMMARY

In some embodiments, a filamentous fungal cell is provided, comprising an inactivated pepA gene and a recombinant gene comprising a PEPA homolog selected from the group consisting of pepAa, pepAb, pepAc, and pepAd. In some embodiments, the recombinant gene comprises a nucleotide sequence selected from the group consisting of SEQ ID NOs: 7 (pepAa amplicon), 9 (pepAa*amplicon), 12 (pepAb amplicon), 17 (pepAc amplicon), 25 (pepAd amplicon), 20 (pepAd amplicon), and 27 (pepAd** amplicon). In some embodiments, the recombinant gene comprises a promoter sequence and a terminator sequence. In some embodiments, the recombinant gene comprises an A. niger glucoamylase promoter sequence, a pepA homolog sequence, and an A. tubingensis glucoamylase terminator sequence. In some embodiments, the recombinant gene is heterologously integrated into the chromosomal DNA of the cell.

In some embodiments of the filamentous fungal cell, the pepA homolog encodes a polypeptide secreted by the cell. In some embodiments, the pepA homolog encodes a polypeptide having at least 85% identity to a sequence selected from group consisting of SEQ ID NOs: 1 (PEPAa), 2 (PEPAb), 3 (PEPAc), 4 (PEPAd), 21 (PEPAd*), 23 (PEPAa*), and 28 (PEPAd**).

In some embodiments, the filamentous fungal cell secretes the polypeptide encoded by the pepA homolog in an amount at least about 10% to about 1000% greater than the corresponding parent strain. In some embodiments, the polypeptide encoded by the pepA homolog is at least about 5%, at least about 10%, at least about 15%, at least about 20%, at least about 25%, or even more, of the total protein secreted by the filamentous fungal cell.

In some embodiments of the filamentous fungal cell, the protease activity of a culture supernatant of the cell is at least about 0.5-fold to about 100-fold greater than the protease activity of a culture supernatant of a corresponding parent strain. In some embodiments, the protease activity of the PEPA homolog is greater at about pH 2.0 than at about pH 3.0.

In some embodiments, the filamentous fungal cell is selected from the group consisting of an Aspergillus spp., a Rhizopus spp., a Trichoderma spp., and a Mucor spp. In some embodiments, the filamentous fungus is an Aspergillus sp. selected from the group consisting of A. oryzae, A. niger, A. awamori, A. nidulans, A. sojae, A. japonicus, A. kawachi and A. aculeatus.

In some embodiments, a method for producing an acidic protease is provided, comprising: (a) providing a filamentous fungal cell comprising an inactivated pepA gene; (b) transforming the cell with a recombinant gene comprising a pepA homolog selected from the group consisting of pepAa, pepAb, pepAc, and a truncated pepAd; and (c) growing the cell under conditions suitable for expressing the recombinant gene. In some embodiments, the method further comprises recovering the acidic protease. In some embodiments of the method, the recombinant gene comprises a nucleotide sequence selected from the group consisting of SEQ ID NOs: 7, 9, 12, 17, 20, 25, and 27. In some embodiments, the recombinant gene comprises an A. niger glucoamylase promoter sequence, a pepA homolog sequence, and an A. tubingensis glucoamylase terminator sequence.

In some embodiments, an isolated nucleic acid is provided, comprising an A. niger glucoamylase promoter sequence, a pepA homolog sequence, and an A. tubingensis glucoamylase terminator sequence, wherein the pepA homolog sequence encodes a polypeptide having at least 85% identity to an amino acid sequence selected from the group consisting of SEQ ID NOs: 1, 2, 3, 4, 21, 23, and 28. In some embodiments of the isolated nucleic acid, the pepA homolog comprises a nucleotide sequence selected from the group consisting of SEQ ID NOs: 7, 12, 17, and 20.

In some embodiments, a method for producing an acidic protease is provided, comprising: a) introducing a nucleic acid into a filamentous fungal cell, wherein the cell comprises an inactivated pepA gene and wherein the nucleic acid comprises a promoter sequence, a pepA homolog sequence, and a terminator sequence; and b) growing the cell under conditions suitable for producing the acidic protease. In some embodiments, the method is carried out wherein introducing the nucleic acid into the filamentous fungal cell comprises transforming the cell with a vector, and in some embodiments wherein the vector is a plasmid. In some embodiments, the plasmid introducted is selected from the group consisting of pGAMD-pepAa, pGAMD-pepAb, pGAMD-pepAc, pGAMD-pepAd, pGAMD-pepAd*, pGAMD-pepAa*, and pGAMD-pepAd**.

In some embodiments, a vector is provided, comprising an A. niger glucoamylase promoter sequence, a pepA homolog sequence, and an A. tubingensis glucoamylase terminator sequence, wherein the pepA homolog sequence encodes a polypeptide having at least 85% identity to a sequence selected from the group consisting of SEQ ID NOs: 1, 2, 3, 4, 21, 23, and 28. In some embodiments, the vector comprises a nucleotide sequence selected from the group consisting of SEQ ID NOs: 7, 9, 12, 17, 20, 25, and 27. In some embodiments, the vector is a plasmid, and in some embodiments the plasmid comprises the pGAMD plasmid with a pepA homolog sequence insert. In some embodiments, the plasmid is selected from the group consisting of pGAMD-pepAa, pGAMD-pepAb, pGAMD-pepAc, pGAMD-pepAd, pGAMD-pepAd*, pGAMD-pepAa*, and pGAMD-pepAd**.

In some embodiments, a method for producing an acidic protease is provided, comprising: (a) transforming a filamentous fungal cell with a plasmid, wherein the cell comprises an inactivated native pepA gene and wherein the plasmid comprises an A. niger glucoamylase promoter sequence, a pepA homolog sequence, and an A. tubingensis glucoamylase terminator sequence; and (b) growing the cell under conditions suitable for producing the protein encoded by the pepA homolog sequence. In some embodiments, the method further comprises recovering the acidic protease. In some embodiments of the method, the pepA homolog sequence encodes a polypeptide having at least 85% identity to a sequence selected from the group consisting of SEQ ID NOs: 1, 2, 3, 4, 21, 23, and 28. In one embodiment, the plasmid comprises a nucleotide sequence selected from the group consisting of SEQ ID NOs: 7, 9, 12, 17, 20, 25, and 27.

In some embodiments, an isolated nucleic acid encoding a polypeptide having at least 85%, at least 90%, at least 91%, at least 92%, at least 93%, at least 94%, at least 95%, at least 96%, at least 97%, at least 98%, at least 99%, or even greater identity to SEQ ID NO: 21, is provided.

In some embodiments, an isolated polypeptide having at least 85%, at least 90%, at least 91%, at least 92%, at least 93%, at least 94%, at least 95%, at least 96%, at least 97%, at least 98%, at least 99%, or even greater identity to SEQ ID NO: 21, is provided.

In some embodiments, an enzyme composition comprising a polypeptide having at least 85%, at least 90%, at least 91%, at least 92%, at least 93%, at least 94%, at least 95%, at least 96%, at least 97%, at least 98%, at least 99%, or even greater identity to SEQ ID NO: 21, is provided.

### BRIEF DESCRIPTION OF THE DRAWINGS

FIG. 1 depicts the PEPAa amino acid sequence (SEQ ID NO: 1).

FIG. 2 depicts the PEPAb amino acid sequence (SEQ ID NO: 2).

FIG. 3 depicts the PEPAc amino acid sequence (SEQ ID NO: 3).

FIG. 4 depicts the PEPAd amino acid sequence (SEQ ID NO: 4).

FIG. 5 depicts the nucleotide sequence of the pepAa amplicon (SEQ ID NO: 7), which comprises the pepAa gene nucleotide sequence encoding PEPAa (SEQ ID NO: 1).

FIG. 6 depicts the pGAMD vector, which comprises the A. niger glucoamylase promoter sequence and an A. tubingensis glucoamylase terminator sequence.

FIG. 7 depicts the 10.4 kb pGAMD-pepAa vector, which comprises the A. niger glucoamylase promoter sequence, the pepAa sequence, and the A. tubingensis glucoamylase terminator sequence.

FIG. 8 depicts an SDS-PAGE gel image of supernatant from pGAMD-pepAa transformed A. niger GAP3 strain and non-transformed GAP3 parent strain (far right lane). The circled broad band at −55 kD, which is not present in the GAP3 control lanes, is attributed to the recombinant produced PEPAa enzyme secreted by the cells into the supernatant. The breadth/heterogeneity of the recombinant PEPAa bands is believed to be due to glycosylation of the protein.

FIG. 9 depicts the nucleotide sequence of the pepAa* (“truncated pepAa”) amplicon (SEQ ID NO: 9), which comprises the pepAa* nucleotide sequence.

FIG. 10 depicts the nucleotide sequence of the pepAb amplicon (SEQ ID NO: 12), which comprises the pepAb gene nucleotide sequence encoding PEPAb (SEQ ID NO: 2).

FIG. 11 depicts the 10.3 kb pGAMD-pepAb vector, which comprises the A. niger glucoamylase promoter sequence, the pepAb sequence, and the A. tubingensis glucoamylase terminator sequence.

FIG. 12 depicts an SDS-PAGE gel image of supernatant from spore-purified strain #1-3 of the pGAMD-pepAb transformed A. niger GAP3 strain and non-transformed GAP3 parent strain (far right lane). The circled band at −47 kD, which is not present in the GAP3 control lane, is attributed to the recombinant produced PEPAb enzyme secreted into the supernatant.

FIG. 13 depicts the nucleotide sequence of the pepAc amplicon (SEQ ID NO: 17), which comprises the pepAc gene nucleotide sequence encoding PEPAc (SEQ ID NO: 3).

FIG. 14 depicts the 10.4 kb pGAMD-pepAc vector, which comprises the A. niger glucoamylase promoter sequence, the pepAc sequence, and the A. tubingensis glucoamylase terminator sequence.

FIG. 15 depicts an SDS-PAGE gel image of supernatant from spore-purified strain #12-2 of the pGAMD-pepAc transformed A. niger GAP3 strain and non-transformed GAP3 parent strain (far right lane). The circled broad band at ˜60 kD, which is not present in the GAP3 control lane, is attributed to the recombinant produced PEPAc enzyme secreted into the supernatant.

FIG. 16 depicts the nucleotide sequence of the pepAd* (i.e., “truncated pepAd”) amplicon (SEQ ID NO: 20), which comprises the pepAd* gene nucleotide sequence encoding PEPAd* (SEQ ID NO: 21).

FIG. 17 depicts the amino acid sequence of the PEPAd* (i.e., “truncated pepAd”) protein (SEQ ID NO: 21) and the C-terminal GPI anchor sequence (SEQ ID NO: 22) of PEPAd that is deleted to form PEPAd*.

FIG. 18 depicts the 10.2 kb pGAMD-pepAd'vector, which comprises the A. niger glucoamylase promoter sequence, the pepAd* sequence, and the A. tubingensis glucoamylase terminator sequence.

FIG. 19 depicts an SDS-PAGE gel image with lanes including supernatant from the four distinct spore-purified strains of A. niger engineered to overproduce recombinant PEPAd* proteins, as well as the GAP3 control and wild-type A. niger 13528 strain (CGMCC No. AS3.10145). The lane labeled Ad#9-2 is the spore purified strain of the pGAMD-pepAd* transformed A. niger GAP3 strain. The circled broad band at ˜60 kD, which is not present in the GAP3 control lane is, attributed to the recombinantly produced PEPAd* enzyme secreted into the supernatant.

FIG. 20 depicts the nucleotide sequence of the pepAd amplicon (SEQ ID NO: 25), which comprises the full-length pepAd gene nucleotide sequence encoding PEPAd (SEQ ID NO: 4).

FIG. 21 depicts a plot of the protease activity (measured using casein proteolysis assay described in Example 7) of wild-type 13528 strain (CGMCC No. AS3.10145), GAP3 strain, and the spore-purified A. niger strains containing the recombinant genes: pepAa, pepAb, pepAc, and pepAd*.

FIG. 22 depicts plots of protease activity (measured using casein proteolysis assay described in Example 7) versus pH for: (A) wild-type 13528 strain; (B) GAP3 strain; (C) strain PepAa#2-9; (D) strain PepAb#1-3; (E) strain PepAc#12-2; and (F) strain PepAd49-2.

FIG. 23 depicts plots of protease activity (measured using casein proteolysis assay described in Example 7) versus temperature for: (A) wild-type 13528 strain; (B) GAP3 strain; (C) strain PepAa#2-9; (D) strain PepAb#1-3; (E) strain PepAc#12-2; and (F) strain PepAd49-2.

FIG. 24 depicts the PEPAa* amino acid sequence (SEQ ID NO: 23).

FIG. 25 depicts the depicts the nucleotide sequence of the pepAd** amplicon (SEQ ID NO: 24), which comprises the pepAd** nucleotide sequence encoding mutant PEPAd** protein (SEQ ID NO: 28).

FIG. 26 depicts the PEPAd** amino acid sequence (SEQ ID NO: 28).

FIG. 27 depicts an SDS-PAGE gel image with lanes including supernatant from the three distinct spore-purified strains of A. niger engineered to overproduce recombinant PEPAd* (lane 1), PEPAd (lane 2) and PEPAd** proteins (lane 3). Corresponding protein bands are indicated by arrows.

FIG. 28 depicts a plot of the protease activity (measured using casein proteolysis assay described in Example 7) of wild-type GAP3 strain, the spore-purified A. niger strains producing recombinant PEPAd* (3# and 9#), PEPAd (5# and 8#) and PEPAd** (3# and 7#).

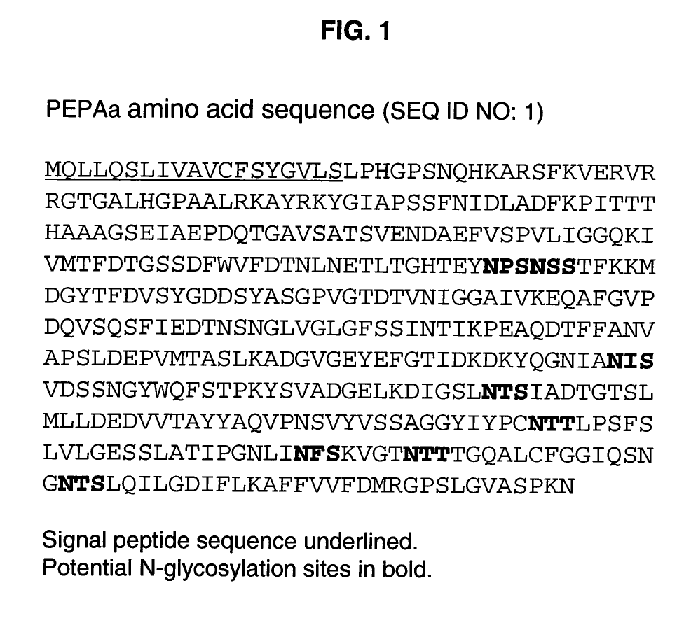

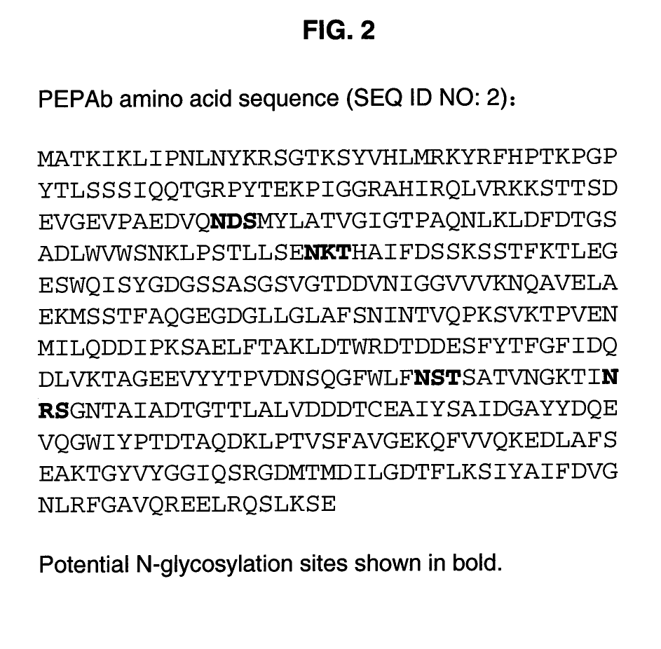

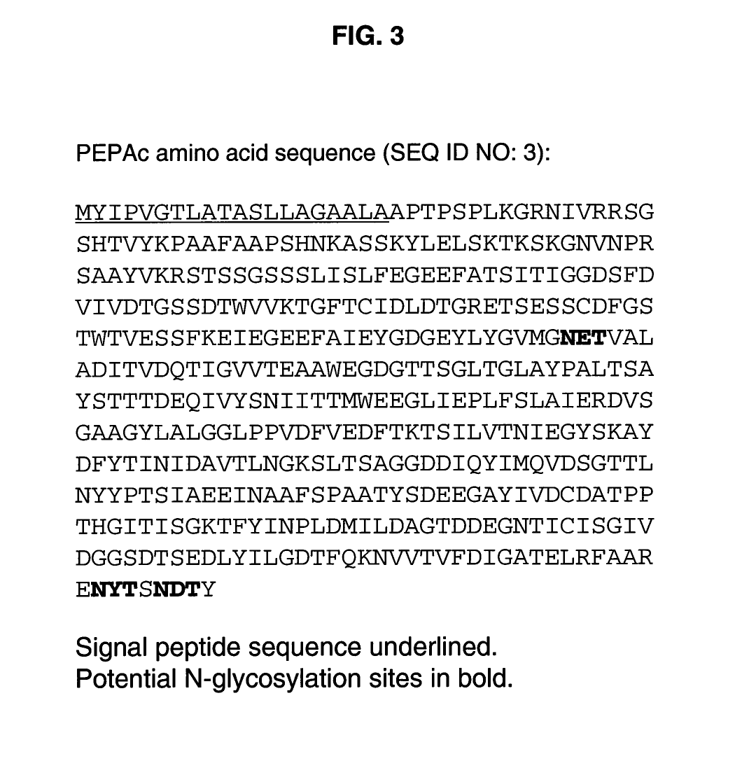

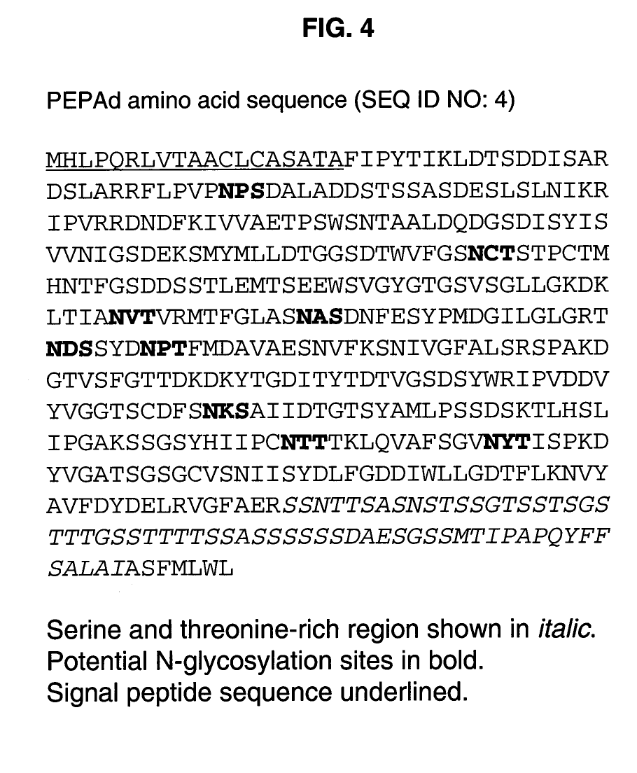

### DETAILED DESCRIPTION

### I. Overview

The present compositions and methods relate to a filamentous fungal cell, such as a cell of an Aspergillus sp., having an inactivated pepA aspartic protease gene and an integrated recombinant gene that is a homolog of pepA selected from pepAa, pepAb, pepAc, and pepAd, wherein the cell produces the aspartic protease encoded by the recombinant pepA homolog. Such a filamentous fungal cell secretes the recombinant encoded PEPA homolog enzyme in an amount that is substantially greater than the amount of aspartic protease produced by the corresponding parental cell/strain. In some embodiments, the production of the PEPA homolog enzyme, measured as casein proteolytic activity of a cell culture supernatant, is at least about 0.5-fold to about 100-fold greater than the corresponding parent cell/strain.

In some embodiments, the integrated recombinant gene encoding the PEPA homolog enzyme comprises an A. niger glucoamylase promoter sequence, the pepA homolog sequence, and an A. tubingensis glucoamylase terminator sequence. In some embodiments, the integrated recombinant gene comprises a nucleotide sequence selected from SEQ ID NOs: 7, 9, 12, 17, 20, 25, and 27.

The present compositions and methods provide a plasmid vector comprising a recombinant gene encoding the PEPA homolog enzyme comprises an A. niger glucoamylase promoter sequence, the pepA homolog sequence, and an A. tubingensis glucoamylase terminator sequence. In some embodiments, the recombinant gene included in the plasmid vector comprises a nucleotide sequence selected from SEQ ID NOs: 7, 9, 12, 17, 20, 25, and 27. The plasmid vector can be used to transform filamentous fungal cells (e.g., Aspergillus sp.) resulting in cells with an integrated recombinant gene comprising a pepA homolog sequence that is capable of producing substantially greater amounts of a PEPA homolog enzyme than the corresponding parent strain.

The present compositions and methods also provide a truncated version of the pepA homolog gene, pepAd. This truncated pepAd gene (i.e., “pepAd*”) has deleted a portion of its 3′ sequence that encodes a C-terminal GPI anchor sequence (SEQ ID NO: 22). Unlike the full-length protein PEPAd (SEQ ID NO: 4), the truncated PEPAd* enzyme (SEQ ID NO: 21) is secreted by a transformed filamentous fungal cell.

### II. Definitions

All patents and publications referred to herein, including all sequences disclosed in such patents and publications, are expressly incorporated by reference. Unless otherwise defined, all technical and scientific terms used herein have the same meaning as commonly understood by one of ordinary skill in the art to which the compositions and methods belong (see, e.g., Singleton et al., DICTIONARY OF MICROBIOLOGY AND MOLECULAR BIOLOGY, 2D ED., John Wiley and Sons, New York (1994); and Hale and Marham, THE HARPER COLLINS DICTIONARY OF BIOLOGY, Harper Perennial, N.Y. (1991), both of which provide one of skill with a general dictionary of many of the terms used herein). Any methods and materials similar or equivalent to those described herein can be used in the practice or testing of the present compositions and methods.

It is intended that every disclosed maximum (or minimum) numerical limitation includes every lower (or higher) numerical limitation, as if such lower (or higher) numerical limitations were expressly described. Moreover, every disclosed numerical range is intended to include every narrower numerical range that falls within such broader numerical range, as if such narrower numerical ranges were expressly described.

In some aspects, the present compositions and methods rely on routine techniques and methods used in the fields of genetic engineering and molecular biology. The following resources include descriptions of general methodology useful in accordance with the present description: Sambrook et al., MOLECULAR CLONING: A LABORATORY MANUAL (2nd Ed., 1989); Kreigler, GENE TRANSFER AND EXPRESSION; A LABORATORY MANUAL (1990) and Ausubel et al., (eds.) CURRENT PROTOCOLS IN MOLECULAR BIOLOGY (1994). These general references provide definitions and methods known to those in the art. However, it is not intended that the present compositions and methods be limited to any particular methods, protocols, and reagents described, as these may vary.

As used herein, the singular articles “a,” “an,” and “the” include the plural referents unless context clearly dictates otherwise. Thus, for example, reference to “a host cell” includes a plurality of such host cells.

As used herein, the phrase “at least,” when used in combination with a list of values or terms is meant to apply to each value or term in the list. For example, the phrase “at least 85%, 90%, 95% and 99% sequence identity” is used to denote at least 85%, at least 90%, at least 95% and/or at least 99% sequence identity.

As used herein, the term “comprising” and its cognates are used in their inclusive sense; that is, equivalent to the term “including” and its corresponding cognates.

Unless otherwise indicated, nucleic acids are written left to right in 5′ to 3′ orientation; amino acid sequences are written left to right in amino to carboxyl orientation, respectively.

Headings are not intended as limitations, and the various aspects or embodiments described under one heading may apply to the description as a whole.

As used herein, when describing proteins and genes that encode them, the term for the gene is generally italicized, (e.g., the gene that encodes A. niger PEPA aspartic protease may be denoted as “pepA”). The term for the protein is generally not italicized and all letters are generally capitalized, (e.g., the protein encoded by the pepA gene may be denoted as “PEPA”).

The following terms are defined for clarity:

As used herein, the term “derived from” encompasses the terms “originated from,” “obtained from,” “obtainable from,” and “isolated from,” and indicates that a polypeptide encoded by a nucleotide sequence is produced from a cell in which the nucleotide is naturally present or in which the nucleotide sequence has been inserted.

As used herein, the terms “aspartic protease” or “aspartic proteinase” refer to the pepsin-like protease enzymes that are members of the A1 family of peptidases (see, e.g., Rawlings et al., “MEROPS: the peptidase database,” Nucleic Acids Res. 32:160-64 (2004). The term “aspartic protease” includes but is not limited to the enzymes encoded by the A. niger genes: pepA, pepAa, pepAb, pepAc, and pepAd.

As used herein, the term “PEPAx” refers to proteins encoded by homologs of the pepA gene including but not limited to the A. niger homologs: pepAa, truncated pepAa (pepAa*), pepAb, pepAc, pepAd, and truncated pepAd (pepAd* and pepAd**).

As used herein, the term “truncated,” such as “truncated PepAx,” refers to an enzyme, wherein at least part of the amino acid sequence has been eliminated, but the remaining portion retains at least some catalytic function.

As used herein, the terms “native” or “endogenous,” with reference to a polynucleotide or protein, refers to a polynucleotide or protein that occurs naturally in a subject host cell.

As used herein, the term “recombinant” used with reference to a cell, nucleic acid, or protein, indicates that the cell, nucleic acid, or protein has been modified by the introduction of a native or heterologous nucleic acid or protein using a vector, or derived from a cell so modified. Thus, the terms “recombinant PEPAx,” “recombinantly expressed PEPAx” and “recombinant(ly) produced PPEAx” refer to a mature PEPAx protein sequence that is produced in a host cell from the expression of a pepAx gene introduced into the cell.

As used herein, the term “vector” refers to any nucleic acid molecule into which another nucleic acid molecule (e.g., a gene) can be inserted and which can be introduced into and replicate within cells. Thus, the term refers to any nucleic acid construct (and, if necessary, any associated delivery system) capable of use for transferring of genetic material between different host cells. Many prokaryotic and eukaryotic vectors are commercially available. Selection of appropriate vectors is within the knowledge of those having skill in the art.

As used herein, the term “plasmid” refers to a circular double-stranded (ds) DNA construct that can be used as a vector for introducing DNA into a cell. Plasmids act as extrachromosomal self-replicating genetic element in many bacteria and some eukaryotes. In some embodiments, one or more plasmids can be integrated into the genome of the host cell into which it is introduced.

As used herein, the terms “host,” “host cell,” or “host strain” refer to a cell that can express a DNA sequence introduced into the cell. Exemplary host cells are those of an Aspergillus sp.

As used herein, the term “filamentous fungal cell” refers to a cell of any of the species of microscopic filamentous fungi that grow as multicellular filamentous strands including but not limited to: Aspergillus spp. (e.g., A. oryzae, A. niger, A. kawachi, and A. awamori), Trichoderma spp. (e.g., Trichoderma reesei (previously classified as T. longibrachiatum and currently also known as Hypocrea jecorina), Trichoderma viride, Trichoderma koningii, Trichoderma harzianum); Penicillium spp., Humicola spp. (e.g., Humicola insolens and Humicola grisea); Chrysosporium spp. (e.g., C. lucknowense), Gliocladium spp., Fusarium spp., Neurospora spp., Hypocrea spp., Rhizopus spp., Mucor spp., and Emericella spp. (see also, Innis et al., (1985) Science 228:21-26).

As used herein, the term “filamentous fungi” refers to all filamentous forms of the subdivision Eumycotina (see, Alexopoulos, C. J. (1962), INTRODUCTORY MYCOLOGY, Wiley, N.Y. and AINSWORTH AND BISBY DICTIONARY OF THE FUNGI, 9th Ed. (2001) Kirk et al., Eds., CAB International University Press, Cambridge UK). These fungi are characterized by a vegetative mycelium with a cell wall composed of chitin, cellulose, and other complex polysaccharides. The filamentous fungi are morphologically, physiologically, and genetically distinct from yeasts. Vegetative growth by filamentous fungi is by hyphal elongation and carbon catabolism is obligatory aerobic.

As used herein, “Aspergillus” or “Aspergillus spp.” includes all species within the genus “Aspergillus,” as known to those of skill in the art, including but not limited to A. niger, A. oryzae, A. awamori, A. kawachi and A. nidulans.

As used herein, the term “Trichoderma” or “Trichoderma spp.” refer to any fungal genus previously or currently classified as Trichoderma.

As used herein, the term “inactivated” in the context of a cell refers to a host organism (e.g., Aspergillus niger cells) having one or more inactivated genes. The term is intended to encompass progeny of an inactivated mutant or inactivated strain and is not limited to the cells subject to the original inactivation means (e.g., the initially transfected cells).

As used herein, the term “inactivation” refers to any method that substantially prevents the functional expression of one or more genes, fragments or homologues thereof, wherein the gene or gene product is unable to exert its known function. It is intended to encompass any means of gene inactivation include deletions, disruptions of the protein-coding sequence, insertions, additions, mutations, gene silencing (e.g., RNAi genes, antisense nucleic acids, etc.), and the like. Accordingly, the term “inactivated” refers to the result of “inactivation” as described above. In some embodiments, “inactivation” results in a cell having no detectable activity for the gene or gene product corresponding to the inactivated gene. In some embodiments, “inactivation” results in little or no functional expression of a gene but still functional expression of a homolog to the gene. Consequently, an “inactivated strain” may exhibit a partially active phenotype due to the homolog gene.

As used herein, the term “corresponding parent(al) strain” refers to the host strain from which an inactivated mutant is derived (e.g., the originating and/or wild-type strain). A corresponding parent strain can include a strain that has been engineered to include an inactivated gene, e.g., the GAP3 strain of A. niger which has an inactivated pepA gene.

As used herein the term “gene” means a segment of DNA involved in producing a polypeptide and can include regions preceding and following the coding regions (e.g., promoter, terminator, 5′ untranslated (5′ UTR) or leader sequences and 3′ untranslated (3′ UTR) or trailer sequences, as well as intervening sequence (introns) between individual coding segments (exons)).

As used herein, the terms “homolog,” “gene homolog,” or “homologous gene,” refer to a gene which has a homologous sequence and results in a protein having an identical or similar function. The terms encompasse genes that are separated by speciation (i.e., the development of new species) (e.g., orthologs or orthologous genes), as well as genes that have been separated by genetic duplication (e.g., paralogs or paralogous genes).

As used herein, the term “homologous sequence” refers to a nucleotide or polypeptide sequence having at least about 60%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or even greater sequence identity to a subject nucleotide or amino acid sequence when optimally aligned for comparison. In some embodiments, homologous sequences have between about 80% and 100% sequence identity, in some embodiments between about 90% and 100% sequence identity, and in some embodiments, between about 95% and 100% sequence identity.

Sequence homology can be determined using standard techniques known in the art (see, e.g., Smith and Waterman, Adv. Appl. Math., 2:482 (1981); Needleman and Wunsch, J. Mol. Biol., 48:443 (1970); Pearson and Lipman, Proc. Natl. Acad. Sci. USA 85:2444 (1988); programs such as GAP, BESTFIT, FASTA, and TFASTA in the Wisconsin Genetics Software Package (Genetics Computer Group, Madison, Wis.); and Devereux et al., Nucleic Acid Res. 12:387-395 (1984)).

Useful algorithms for determining sequence homology include: PILEUP and BLAST (Altschul et al., J. Mol. Biol., 215:403-10, (1990); and Karlin et al., Proc. Natl. Acad. Sci. USA 90:5873-87 (1993)). PILEUP uses a simplification of the progressive alignment method of Feng and Doolittle (Feng and Doolittle, J. Mol. Evol., 35:351-60 (1987)). The method is similar to that described by Higgins and Sharp (Higgins and Sharp, CABIOS 5:151-53 (1989)). Useful PILEUP parameters including a default gap weight of 3.00, a default gap length weight of 0.10, and weighted end gaps.

A particularly useful BLAST program is the WU-BLAST-2 program (see, Altschul et al., Meth. Enzymol. 266:460-80 (1996)). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. An amino acid sequence % identity value is determined by the number of matching identical residues divided by the total number of residues of the “longer” sequence in the aligned region. The longer sequence is the one having the most actual residues in the aligned region (gaps introduced by WU-Blast-2 to maximize the alignment score are ignored).

As used herein, the term “integrated,” used in reference to a gene, means incorporated into the chromosomal DNA of a host cell. Genes can be integrated heterologously or homologously. For example, a recombinant version of a pepA homolog gene native to the host cell is inserted in a plasmid, used to transform the cell, and integrated heterologously into the host cell's chromosomal DNA. Multiple copies of the plasmid recombine heterologously with the chromosomal DNA of filamentous fungal cell and are capable of expression resulting in increased production of the secreted protein in the host cell's culture supernatant. Genes can also be integrated via the process of “homologous recombination,” wherein the homologous regions of the introduced (transforming) DNA align with homologous regions of the host chromosome. Subsequently, the sequence between the homologous regions is replaced by the incoming sequence in a double crossover.

As used herein, the term “promoter” refers to a nucleic acid sequence that functions to direct transcription of a downstream gene. In some embodiments, the promoter is appropriate to the host cell in which a desired gene is being expressed. An “inducible promoter” is a promoter that is active under environmental or developmental regulation.

As used herein, the term “terminator” refers to a nucleic acid sequence located just downstream of the coding segment of a gene that functions to stop transcription of the gene.

As used herein, nucleic acid sequences are “operably linked” when one nucleic acid sequence is placed in a functional relationship with another nucleic acid sequence. For example, DNA encoding a secretory leader (i.e., a signal peptide), is operably linked to DNA encoding a polypeptide if the resulting polypeptide is expressed as a preprotein in which the leader that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “nucleic acid” refers to a nucleotide or polynucleotide sequence, and fragments or portions thereof, as well as to DNA, cDNA, and RNA of genomic or synthetic origin which may be double-stranded or single-stranded, whether representing the sense or antisense strand. It will be understood that as a result of the degeneracy of the genetic code, a multitude of nucleotide sequences may encode a given protein.

As used herein, the terms “DNA construct,” “nucleic acid construct,” “expression cassette,” and “expression vector,” refer to nucleic acid molecules generated by recombinant or synthetic means, which can be introduced into a host cell or organism (i.e., “transformed into a host cell”) and transcribed. For example, a DNA construct can be incorporated into a plasmid used to transform a host cell or organism. The DNA construct may be generated in vitro using PCR or any other suitable technique. The transforming DNA can include a gene to be integrated into a host genome, and/or can include flanking sequences such as promoters, terminators, or homology boxes. The transforming DNA construct can comprise other non-homologous sequences added to the ends (e.g., stuffer sequences or flanks). The ends can be closed such that the transforming DNA construct forms a closed circle (i.e., a plasmid).

As used herein an “amino acid sequence” refers to peptide or protein, or portions thereof. The terms “protein,” “peptide,” and “polypeptide” are used interchangeably to refer to a contiguous chain of amino acid residues linked by peptide bonds.

As used herein, the term “expression” refers to a process by which a polypeptide is produced in a cell or in an in vitro reaction. The process includes both transcription and translation of the gene. The process may also include secretion of the resulting polypeptide.

As used herein, the terms “overproducing” and “overproduction,” with reference to a recombinant cell, refer to a cell that produces and secretes a recombinant protein in an amount of at least about 5% of the total amount of secreted protein.

As used herein, the terms “insertion” and “addition,” in the context of an amino acid or nucleotide sequence, refer to a change in a nucleic acid or amino acid sequence in which one or more amino acid residues or nucleotides are added compared to the endogenous protein product or mRNA/chromosomal sequence.

As used herein, in the context of “introducing a nucleic acid sequence into a cell,” the term “introducing” (and in past tense, “introduced”) refers to any method suitable for transferring the nucleic acid sequence into the cell, including but not limited to transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, agrobacterium mediated transformation, and protoplast fusion.

As used herein, “an incoming sequence” refers to a DNA sequence that is being introduced into a host cell. The incoming sequence can be a DNA construct, can encode one or more proteins of interest (e.g., a recombinant version of a native protein), can include flanking sequences such as a promoter and terminator around a protein of interest, can be a functional or non-functional gene and/or a mutated or modified gene, and/or can be a selectable marker gene(s). For example, the incoming sequence can include a truncated version of the pepAd gene.

As used herein, a “flanking sequence” or “flanking region” refers to any sequence that is either upstream or downstream of the sequence being discussed (e.g., for genes A-B-C, gene B is flanked by the A and C gene sequences). The incoming sequence may be a sequence encoding a protein and the flanking sequences are a promoter and a terminator sequence. The incoming sequence may be flanked by a homology box on each side. The incoming sequence and the homology boxes may comprise a unit that is flanked by stuffer sequence on each side. A flanking sequence may be present on only a single side (e.g., either 5′ or 3′) or on each side of the sequence being flanked. The sequence of each homology box is preferably homologous to a sequence in the Aspergillus chromosome. These sequences direct where in the Aspergillus chromosome the new construct becomes integrated and what part of the Aspergillus chromosome will be replaced by the incoming sequence. These sequences may direct where in the Aspergillus chromosome the new construct becomes integrated without any part of the chromosome being replaced by the incoming sequence. The 5′ and 3′ ends of a selective/selectable marker may be flanked by a polynucleotide sequence comprising a section of the inactivating chromosomal segment. A flanking sequence may be present on only a single side (e.g., either 5′ or 3′), or present on each side of the sequence being flanked. As used herein, the terms “selectable marker” and “selective marker” refer to a nucleic acid capable of expression in host cell, which allows for ease of selection of those hosts containing the marker. Thus, the term “selectable marker” refers to genes that provide an indication that a host cell has taken up (e.g., has been successfully transformed with) an incoming nucleic acid of interest (e.g., inactivated gene) or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation. Selective markers useful with the present compositions and methods include, but are not limited to, antimicrobial resistance markers (e.g., ampR, phleoR, specR, kanR, eryR, tetR, cmpR, hygroR, and neoR; see e.g., Guerot-Fleury, Gene, 167:335-37 (1995); Palmeros et al., Gene 247:255-64 (2000); and Trieu-Cuot et al., Gene, 23:331-41 (1983)), auxotrophic markers, such as tryptophan, pyrG and amdS, and detection markers, such as β-galactosidase.

As used herein, the term “hybridization” refers to the process by which a strand of nucleic acid joins with a complementary strand through base pairing, as known in the art.

A nucleic acid sequence is considered to be “selectively hybridizable” to a reference nucleic acid/nucleotide sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while an intermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denaturated sheared salmon sperm DNA, followed by washing the filters in 1×SSC at about 37-50° C. Those of skill in the art know how to adjust the temperature, ionic strength, etc., as necessary to accommodate factors such as probe length and the like.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced (i.e., in the presence of nucleotides and an inducing agent such as DNA polymerase and at a suitable temperature and pH). Usually, the primer is single stranded for maximum efficiency in amplification. Most often, the primer is an oligodeoxyribonucleotide.

As used herein, the term “polymerase chain reaction (PCR)” refers to methods for amplifying DNA strands using a pair of primers, DNA polymerase, and repeated cycles of DNA polymerization, melting, and annealing (see, e.g., U.S. Pat. Nos. 4,683,195 4,683,202, and 4,965,188, which are hereby incorporated by reference herein).

As used herein, the term “restriction enzyme” refers to a bacterial enzyme, which cuts double-stranded DNA at or near a specific nucleotide sequence.

As used herein, a “restriction site” refers to a nucleotide sequence recognized and cleaved by a given restriction endonuclease and is frequently the site for insertion of DNA fragments. In certain embodiments of the compositions and methods restriction sites are engineered into the selective marker and into 5′ and 3′ ends of the DNA construct.

As used herein, the terms “isolated” and “purified” are used to refer to a molecule (e.g., a nucleic acid or polypeptide) or other component that is removed from at least one other component with which it is naturally associated.

As used herein, the term “culturing” refers to growing a population of microbial cells under suitable conditions in a liquid or solid medium. In one embodiment, culturing refers to fermentative bioconversion of a starch substrate, such as a substrate comprising granular starch, to an end-product (typically in a vessel or reactor). Fermentation is the enzymatic and anaerobic breakdown of organic substances by microorganisms to produce simpler organic compounds. While fermentation occurs under anaerobic conditions it is not intended that the term be solely limited to strict anaerobic conditions, as fermentation also occurs in the presence of oxygen.

As used herein, the term “contacting” refers to the placing of the respective enzyme(s) in sufficiently close proximity to the respective substrate to enable the enzyme(s) to convert the substrate to the end-product. Those skilled in the art will recognize that mixing solutions of the enzyme with the respective substrates can effect contacting.

As used herein, the term “specific activity” means an enzyme unit defined as the number of moles of substrate converted to product by an enzyme preparation per unit time under specific conditions. Specific activity is expressed as units (U)/mg of protein.

As used herein, the term “enzyme unit” refers to the amount of enzyme that produces a given amount of product per given amount of time under assay conditions. In some embodiments, an enzyme unit refers to the amount of enzyme that produces 1 micromole of product per minute under the specified conditions of the assay.

As used herein, the term “yield” refers to the amount of end-product or desired end-product(s) produced using the described compositions and methods. In some embodiments, the yield is greater than that produced using methods known in the art. In some embodiments, the term refers to the volume of the end product. In some embodiment the term refers to the concentration of the end product.

### III. Compositions and Methods for Increased Production of Aspartic Proteases in Filamentous Fungal Cells

Described are compositions and methods relating to filamentous fungal cells genetically engineered to provide increased production of aspartic proteases, including but not limited to PEPAa, PEPAb, PEPAc, and PEPAd. Various aspects and embodiments of these compositions and methods are to be described.

Recombinant Filamentous Fungal Cells for Production of PEPAx Enzyme

In some embodiments the present compositions and methods relate to a filamentous fungal cell comprising an inactivated native pepA gene and an integrated recombinant gene comprising a pepA homolog selected from the group consisting of pepAa, pepAb, pepAc, and pepAd.

In some embodiments, the integrated recombinant gene comprises a promoter and/or a terminator sequence operably linked to the gene. In some embodiments, the operably linked promoter is an A. niger glucoamylase promoter. In some embodiments, the operably linked terminator is an A. tubingensis glucoamylase terminator.

In some embodiments, the integrated recombinant gene comprises a nucleotide sequence encoding a polypeptide having at least 85% identity to a sequence selected from PEPAa (SEQ ID NO: 1), PEPAb (SEQ ID NO: 2), PEPAc (SEQ ID NO: 3), PEPAd (SEQ ID NO: 4), a truncated PEPAd* (SEQ ID NO: 21), a truncated PEPAa* (SEQ ID NO: 23), and a truncated PEPAd** (SEQ ID NO: 28).

In some embodiments of the compositions and methods, the integrated recombinant gene comprises a nucleotide sequence having sequence identity of at least about 80%, 81%, 82%, 83%, 84%, 85%, 86%, 87%, 88%, 89%, 90%, 91%, 92%, 93%, 94%, 95%, 96%, 97%, 98%, 99%, or higher, to a sequence selected from the group consisting of SEQ ID NOs: 7, 9, 12, 17, 20, 25, and 27.

In one embodiment, the filamentous fungus is selected from the group consisting of an Aspergillus spp., a Rhizopus spp., a Trichoderma spp., and a Mucor spp. In one embodiment, the filamentous fungus is an Aspergillus spp. selected from the group consisting of A. oryzae, A. niger, A. awamori, A. nidulans, A. sojae, A. japonicus, A. kawachi and A. aculeatus.

In some embodiments, the integrated recombinant gene is a homolog of pepA. Gene homologs useful with the present compositions and methods have the same or similar function as pepA (i.e., encode polypeptides having the same or similar function) and share at least about 60%, at least about 70%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or even greater sequence identity.

In some embodiments, in addition to the recombinant pepA homolog gene integrated into the filamentous fungal cell, the cell also includes naturally occurring (i.e., wild-type) versions of pepA homolog genes. For example, pepAa, pepAb, pepAc, and pepAd are all found in the A. niger genome. Typically, however, the protein encoded by the naturally occurring pepA homolog gene is produced and secreted by the cell in only very small amounts (e.g., <1% of the total protein secreted by the cell).

In some embodiments, the recombinant versions of pepA homolog genes integrated into the filamentous fungal cell with inactivated pepA result in overproduction of secreted protein by the cell (e.g., >5% of the total protein secreted by the cell). In some embodiments, the recombinant cells produce the pepA homolog protein (i.e., PepAx) in amounts such that it constitutes at least about 5%, at least about 6%, at least about 7%, at least about 8%, at least about 9%, at least about 10%, at least about 12%, at least about 15%, at least about 20%, or even more of the total protein secreted by the cell.

The present compositions and methods include filamentous fungal cells with additional inactivated genes engineered into them which can provide additional increases in the amount of recombinant pepA homolog protein secreted by the cell. In some embodiments, the filamentous fungal cell may include one or more additional inactivated genes. The additional inactivated genes may include but are not limited to those involved in protein degradation or protein modification, such as proteins in the ER degradation pathway, protease genes, such as secreted serine and aspartic protease genes, glycosylation genes and glycoprotein degradation genes. In some embodiments, the additional inactivated genes may be selected from one or more of the following: derA, derB, htmA, mnn9, mnn10, ochA, dpp4, dpp5, pepF, pepAa, pepAb, pepAc and pepAd. The various coding sequences and functions of these genes, as well as the methods for making and using filamentous fungal cells having one or more them inactivated are described in U.S. Patent Publication No. 2006/0246545, which is hereby incorporated by reference herein (see also, Wang et al., “Isolation of four pepsin-like protease genes from Aspergillus niger and analysis of the effect of disruptions on heterologous laccase expression,” Fungal Genet. Biol. 45:17-27 (2008), which is hereby incorporated by reference).

pepAx Homolog Genes

The pepA homolog genes pepAa, pepAb, pepAc and pepAd of the A. niger genome encode proteins of 424 (SEQ ID NO: 1), 426 (SEQ ID NO: 2), 453 (SEQ ID NO: 3), and 480 (SEQ ID NO: 4) amino acids, respectively. Alignment of the four amino acid sequences encoded by the pepAx genes (i.e., the PEPAx proteins) with PEPA demonstrates that functional regions of these proteins are highly conserved. For example, the active site motif of “Asp101-Thr102-Gly103” (using PEPA numbering). Asp156 in PEPA is not conserved in PEPAc which has a Glu residue at this position as seen in some other aspartic proteases (see, e.g., Capasso et al., “Molecular cloning and sequence determination of a novel aspartic proteinase from Antarctic fish,” Biochim. Biophys. Acta 1387:457-61 (1998)).

These four aspartic protease “pepAx” genes are well conserved throughout the Aspergillus genus. Putative orthologs of pepAa, pepAb, pepAc and pepAd have been identified in A. nidulans, A. oryzae, and A. fumigatus.

Comparison of the four pepAx gene sequences (pepAa, pepAb, pepAc and pepAd) to the predicted aspartic protease sequences listed in the peptidase database (MEROPS) using ClustalW (see, e.g., Thompson et al., “CLUSTALW: improving the sensitivity of progressive multiple sequence alignment through sequence weighting, position-specific gap penalties and weight matrix choice,” Nucleic Acids Res. 11:4673-80 (1994)) indicates that other orthologs are present in other species of filamentous fungi. The PEPAa protease appears to be an ortholog of the pepsin-type aspartic protease previously identified from Talaromyces emersonii (AF439995, unpublished) and Scleotinia sclerotiorum (see, e.g., Poussereau et al., “aspS encoding an unusual aspartyl protease from Sclerotinia sclerotiorum is expressed during phytopathogenesis,” FEMS Microbiol. Lett. 194:27-32 (2001)). PEPAb and PEPAc proteases appear to be orthologs of aspartic proteases (BcAP1 and BcAP5) previously identified from Botryotinia fuckeliana (see, e.g., ten Have et al., “An aspartic proteinase gene family in the filamentous fungus Botrytis cinerea contains members with novel features,” Microbiology 150:2475-89 (2004)). The closest ortholog to PEPAd is aspartyl protease 4 from Coccidioides posadasii (ABA54909, unpublished).

In some embodiments, the filamentous fungal cell comprises a integrated pepA homolog gene, wherein the homolog gene is an ortholog from another species of filamentous fungal cell. For example, the present compositions and methods may provide an A. nidulans cell, wherein the integrated recombinant pepA homolog gene is the pepAa gene from A. niger.

In some embodiments, a pepA gene homolog useful with the present compositions and methods is a native gene in a filamentous fungal cell, wherein the gene encodes a polypeptide having at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or an even greater percentage amino acid sequence identity to a sequence selected from the group consisting of SEQ ID NOs: 1, 2, 3, 4, 21, 23, and 28.

In some embodiments, a homologous nucleotide sequence can be found in a related filamentous fungal species (e.g., Aspergillus niger and Aspergillus oryzae) and has at least about 80%, 81%, 82%, 83%, 84%, 85%, 86%, 87%, 88%, 89%, 90%, 91%, 92%, 93%, 94%, 95%, 96%, 97%, 98%, 99%, or an even greater percentage amino acid sequence identity to a nucleotide sequence selected from the group consisting of SEQ ID NOs: 7, 9, 12, 17, 20, 25, and 27.

Methods for determining homologous sequences from host cells are known in the art and include using a nucleic acid sequence disclosed herein to construct an oligonucleotide probe, said probe corresponding to about 6 to 20 amino acids of the encoded protein. The probe may then be used to clone the homologous gene. The filamentous fungal host genomic DNA is isolated and digested with appropriate restriction enzymes. The fragments are separated and probed with the oligonucleotide probe prepared from the protein degradation sequences by standard methods. A fragment corresponding to the DNA segment identified by hybridization to the oligonucleotide probe is isolated, ligated to an appropriate vector and then transformed into a host to produce DNA clones.

In some embodiments, a gene homolog useful with the present compositions and methods can have a nucleotide sequence encoding in an amino acid sequence differing from PEPA by one or more conservative amino acid replacements. In such embodiments, the conservative amino acid replacements include but are not limited to the groups of glycine and alanine; valine, isoleucine and leucine; aspartic acid and glutamic acid; asparagine and glutamine; serine and threonine; tryptophan, tyrosine and phenylalanine; and lysine and arginine.

Truncation of PEPAd GPI Anchor Sequence

The Aspergillus PEPAd sequences have a conserved C-terminal modification consisting of about 70 residues. This extension contains lengthy stretches of hydrophilic, predominantly serine residues, terminating with roughly 20 hydrophobic residues, which may facilitate extracellular attachment. An algorithm for identifying fungal glycosylphosphatidylinositol (GPI) modification motifs (see, e.g., Eisenhaber et al., “A sensitive predictor for potential GPI lipid modification sites in fungal protein sequences and its application to genome-wide studies for Aspergillus nidulans, Candida albicans, Neurospora crassa, Saccharomyces cerevisiae, and Schizosaccharomyces pombe,” J. Mol. Biol. 337, 243-53 (2004)) predicted that GPI modification in PEPAd occurs at Gly456 with high probability scores of S>15. Moreover, PEPAd contains serine-rich stretches just upstream of the GPI modification site. These stretches might be targets for O-glycosylation, which may facilitate adherence to the extracellular glucan matrix of A. niger.

As demonstrated by the construction and expression of the truncated and non-truncated versions of PEPAd (see Examples 5 and 6, described below) removal of a 62-amino acid C-terminal GPI anchor sequence (SEQ ID NO: 22) results in secretion of a truncated PEPAd (SEQ ID NO: 21) into the cell supernatant. Thus, this C-terminal sequence likely provides a glycosylphosphatidylinositol link to the membrane referred to as a “GPI anchor” (see, e.g., Hamada et al., “Screening for glycosylphosphatidylinositol (GPI)-dependent cell wall proteins in Saccharomyces cerevisiae,” Mol. Gen. Genet. 258:53-59 (1998)).

A PEPAd aspartic protease attached to the cell membrane by a GPI anchor, or embedded in the hyphal matrix, might support various functions such as the maturation of other fungal hydrolytic enzymes, the proteolysis of host cell wall protein in the vicinity of the hyphal tip.

The present compositions and methods provide a truncated PEPAd (“PepAd*”) enzyme, having an amino acid sequence of SEQ ID NO: 21 that can be secreted in large quantities by the host cell, purified, and isolated. It is contemplated that a range of mutations made be produced in the truncated C-terminal sequence of pepAd that can provide a variety of truncated PEPAd proteins with aspartic protease activity.

The present compositions and methods provide a mutant PEPAd (“PEPAd**”) enzyme, having an amino acid sequence of SEQ ID NO: 28 that can be secreted in large quantities by the host cell, purified, and isolated. The mutation comprises of PEPAd** comprises a deletion of the single amino acid, Gly456, located in the GPI anchor sequence. It is contemplated that the deletion at Gly456 can disrupt attachment of the enzyme via a glycosylphosphatidylinositol linkage to the cell membrane. The mutant can thus provide another secreted PEPAd protein with aspartic protease activity.

Host Filamentous Fungal Cells

In the present compositions and methods, the host cell is a filamentous fungal cell (see, e.g., Alexopoulos, C. J. (1962), INTRODUCTORY MYCOLOGY, Wiley, New York). Filamentous fungal cells useful with the present compositions and methods include, but are not limited to: Aspergillus spp., (e.g., A. oryzae, A. niger, A. awamori, A. nidulans, A. sojae, A. japonicus, A. kawachi and A. aculeatus); Rhizopus spp., Trichoderma spp. (e.g., Trichoderma reesei (previously classified as T. longibrachiatum and currently also known as Hypocrea jecorina), Trichoderma viride, Trichoderma koningii, and Trichoderma harzianums)), and Mucor spp. (e.g., M. miehei and M. pusillus). In some embodiments, the host cells are Aspergillus niger cells.

In some embodiments, the filamentous fungal host is selected from the group consisting of Aspergillus spp., Trichoderma spp., Fusarium spp., and Penicillium spp. Filamentous fungal host cells useful with the present compositions and methods include but are not limited to: A. nidulans, A. awamori, A. oryzae, A. aculeatus, A. niger, A. japonicus, T. reesei, T. viride, F. oxysporum, and F. solani.

In some embodiments, the host is a strain of Trichoderma, and particularly a strain of T. reesei. Strains of T. reesei are known and nonlimiting examples include ATCC No. 13631, ATCC No. 26921, ATCC No. 56764, ATCC No. 56765, ATCC No. 56767 and NRRL 15709. In some embodiments, the host strain is a derivative of RL-P37. RL-P37 is described in Sheir-Neiss et al. (1984) Appl. Microbiol. Biotechnology 20:46-53.

In some embodiments, the filamentous fungal host cell is a strain of Aspergillus. Specific Aspergillus strains useful with the present compositions and methods also are disclosed in e.g., Ward et al. (1993) Appl. Microbiol. Biotechnol 39:738-43 and Goedegebuur et al. (2002) Curr Gene 41:89-98. Other useful Aspergillus host strains include without limitation: A. nidulans (Yelton et al. (1984) Proc. Natl. Acad. Sci. USA 81:1470-74; Mullaney et al. (1985) Mol. Gen. Genet. 199:37-45; and Johnston et al. (1985) EMBO J. 4:1307-11); A. niger (Kelly et al. (1985) EMBO J. 4:475-79), A. awamori (NRRL 3112, UVK143f; see e.g., U.S. Pat. No. 5,364,770, which is hereby incorporated by reference), ATCC No. 22342, ATCC No. 44733, ATCC No. 14331 and ATCC No. 11490) and A. oryzae (ATCC No. 11490) and derivative strains thereof. In some embodiments, the Aspergillus strain is a pyrG mutant strain and consequentially requires uridine for growth. In some embodiments, the Aspergillus host strain expresses and produces endogenous aspartic proteases.

In some embodiments, the present compositions and methods may be used with particular strains of Aspergillus niger include ATCC 22342 (NRRL 3112), ATCC 44733, and ATCC 14331 and strains derived there from. In some embodiments, the host cell is capable of expressing a heterologous gene. For example, the host cell may be a recombinant cell, which produces a heterologous protein. In other embodiments, the host is one that overexpresses a protein that has been introduced into the cell.

In some embodiments, the host strain is a mutant strain deficient in one or more protease genes other than pepA. Thus, the present compositions and methods provide mutant strains of filamentous fungal cells that overproduce recombinant PEPAx enzymes, wherein the corresponding parent strain already includes an inactivated pepA gene, and other inactivated protease genes.

In some embodiments, the host filamentous fungal strain may have been previously manipulated through genetic engineering. In some embodiments, the genetically engineered host cell or strain may be a protease deficient strain. In other embodiments, expression of various native genes of the filamentous fungal host cell will have been reduced or inactivated. These genes include, for example genes encoding proteases and cellulolytic enzymes, such as endoglucanases (EG) and exocellobiohydrolases (CBH) (e.g., derA, derB, htmA, mnn9, mnn10, ochA, dpp4, dpp5, pepF, pepAa, pepAb, pepAc, pepAd, cbh1, cbh2, egl1, egl2 and egl3). U.S. Pat. No. 5,650,322 (which is hereby incorporated by reference) discloses derivative strains of RL-P37 having deletions in the cbh1 gene and the cbh2 gene. Reference is also made to U.S. Pat. No. 5,472,864 and PCT publication WO05/001036, each of which is hereby incorporated by reference.

Filamentous Fungal Host Cells With Inactivated pepA

In some embodiments, the present compositions and methods provide a filamentous fungal cell strain with an inactivated pepA gene into which a recombinant gene comprising a pepA homolog gene is integrated. In one embodiment, the filamentous fungal cell is from the GAP3 strain of A. niger which has an inactivated pepA gene (see e.g., Ward et al. Appl. Microbiol. Biotechnol, 39:738-43 (1993)).

Inactivation of the pepA gene in the parent cell can occur via any suitable means, including deletions, substitutions (e.g., mutations), disruptions, insertions in the nucleic acid gene sequence, and/or gene silencing mechanisms, such as RNA interference (RNAi). In one embodiment, the expression product of an inactivated gene is a truncated protein with a corresponding change in the biological activity of the protein. In some embodiments, the inactivation results in a loss of biological activity of the gene. In some embodiments, the biological activity of the inactivated gene in a recombinant fungal cell will be effectively zero (i.e., unmeasurable). In some embodiments, some residual activity may remain, and often will be less than about 25%, 20%, 15%, 10%, 5%, 2%, or even less compared to the biological activity of the same or homologous gene in a corresponding parent strain.

In some embodiments, inactivation is achieved by deletion and in other embodiments inactivation is achieved by disruption of the protein-coding region of the gene. In some embodiments, the gene is inactivated by homologous recombination.

In some embodiments, the deletion may be partial as long as the sequences left in the chromosome render the gene functionally inactive. In some embodiments, a deletion mutant comprises deletion of one or more genes that results in a stable and non-reverting deletion. Flanking regions of the coding sequence may include from about 1 bp to about 500 bp at the 5′ and 3′ ends. In some embodiments, the flanking region may be even larger than 500 bp. The end result is that the deleted gene is effectively non-functional. While not meant to limit the methods used for inactivation in some embodiments, the pepA gene is inactivated by deletion.

In some embodiments, the disruption sequence comprises an insertion of a selectable marker gene into the protein-coding region. Typically, this insertion is performed in vitro by reversely inserting a gene sequence into the coding region sequence of the gene inactivated by cleaving then ligating at a restriction site. Flanking regions of the coding sequence may include about 1 bp to about 500 bp at the 5′ and 3′ ends. The flanking region may be even larger than 500 bp. The DNA constrict aligns with the homologous sequence of the host chromosome and in a double crossover event the translation or transcription of the gene is disrupted. For example, the apsB chromosomal gene is aligned with a plasmid comprising the gene or part of the gene coding sequence and a selective marker. In some embodiments, the selective marker gene is located within the gene coding sequence or on a part of the plasmid separate from the gene. The vector is chromosomally integrated into the host, and the host's gene is thereafter inactivated by the presence of the marker inserted in the coding sequence.

In some embodiments, inactivation of the gene is by insertion in a single crossover event with a plasmid as the vector. For example, the vector is integrated into the host cell chromosome and the gene is inactivated by the insertion of the vector in the protein-coding sequence of the gene or in the regulatory region of the gene.

In alternative embodiments, inactivation results due to mutation of the gene. Methods of mutating genes are well known in the art and include but are not limited to site-directed mutation, generation of random mutations, and gapped-duplex approaches (See e.g., Moring et al. Biotech. 2:646 (1984); Kramer et al. Nucleic Acids Res. 12:9441 (1984); and U.S. Pat. No. 4,760,025, which is hereby incorporated by reference).

Recombinant DNA Constructs

In some embodiments, the present compositions and methods includes a DNA construct comprising an incoming sequence that is a pepA homolog sequence. The DNA construct is assembled in vitro, followed by direct cloning of the construct into a competent host (e.g., an Aspergillus host), such that the DNA construct is integrated into the host chromosome. For example, PCR fusion and/or ligation can be employed to assemble a DNA construct in vitro.

In some embodiments, the DNA construct is incorporated into a vector which is a plasmid. In some embodiments, circular plasmids are used. In some embodiments, circular plasmids are designed to be linearized upon contacting with an appropriate restriction enzyme.

In some embodiments, the incoming sequence comprises a pepA homolog gene sequence. A homologous sequence is a nucleic acid sequence encoding a protein having similar or identical function to PEPA and having at least about 60%, at least about 70%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or even greater nucleotide sequence identity to the pepA homolog gene.

In some embodiments, the DNA construct comprises a pepA homolog and flanking sequences include a range of about 1 bp to 2500 bp, about 1 bp to 1500 bp, about 1 bp to 1000 bp, about 1 bp to 500 bp, and 1 bp to 250 bp.

In some embodiments, the DNA construct comprises a pepA homolog and a selective marker. In one embodiment, the A. nidulans amdS gene provides a selectable marker system for the transformation of filamentous fungi. The amdS gene codes for an acetamidase enzyme deficient in strains of Aspergillus and provides positive selective pressure for transformants grown on acetamide media. The amdS gene can be used as a selectable marker even in fungi known to contain an endogenous amdS gene or homolog, e.g., in A. nidulans (Tilburn et al. 1983, Gene 26: 205-221) and A. oryzae (Gomi et al. 1991, Gene 108: 91-98). Background amdS activity of non-transformants can be suppressed by the inclusion of CsCl in the selection medium.

Methods for using amdS marker system in the transformation of industrially important filamentous fungi are established in the art (e.g., in Aspergillus niger (see e.g., Kelly and Hynes (1985) EMBO J. 4: 475-79; Wang et al. (2008) Fungal Genet. Biol. 45:17-27; in Penicillium chrysogenum (see, e.g., Beri and Turner (1987) Curr. Genet. 11:639-41); in Trichoderma reesei (see, e.g., Pentilla et al. (1987) Gene 61:155-64); in Aspergillus oryzae (see, e.g., Christensen et al. (1988) Bio/technology 6:1419-22); in Trichoderma harzianum (see, e.g., Pe'er et al. (1991) Soil Biol. Biochem. 23:1043-46); and U.S. Pat. No. 6,548,285, each of which is hereby incorporated by reference).

In one embodiment, the DNA construct comprising the pepA homolog sequence is incorporated in a vector (e.g., in a plasmid) used to transform the filamentous fungal cell. Typically, the DNA construct is stably transformed resulting in chromosomal integration of the pepA homolog gene which is non-revertable.

Methods for in vitro construction and insertion of DNA constructs into suitable vectors for introduction into host cells are well known in the art. Insertion of sequences is generally accomplished by ligation at convenient restriction sites. If such sites do not exist, synthetic oligonucleotide linkers can be prepared and used in accordance with conventional practice. (see, e.g., Sambrook (1989) supra, and Bennett and Lasure, MORE GENE MANIPULATIONS IN FUNGI, Academic Press, San Diego (1991) pp 70-76). Additionally, vectors can be constructed using known recombination techniques (e.g., Invitrogen Life Technologies, Gateway Technology).

Examples of suitable expression and/or integration vectors that may be used in the practice of the compositions and methods are provided in Sambrook et al. (1989) supra, ss Ausubel (1987) supra, van den Hondel et al. (1991) in Bennett and Lasure (eds.) MORE GENE MANIPULATIONS IN FUNGI, Academic Press pp. 396-428 and U.S. Pat. No. 5,874,276. Exemplary vectors useful with the present compositions and methods include pBS-T, pFB6, pBR322, pUC18, pUC100 and pENTR/D.

In some embodiments, at least one copy of a DNA construct is integrated into the host chromosome. In some embodiments, multiple copies of a DNA construct comprising a pepA homolog are integrated into the host chromosome.

Vectors

A DNA construct comprising nucleic acid encoding a pepA homolog gene encompassed by the compositions and methods may be constructed to transfer a pepA homolog into a host cell. In one embodiment, the DNA construct is transferred into a host filamentous fungal cell using a vector which comprises regulatory sequences operably linked to a sequence encoding a PEPAx aspartic protease.

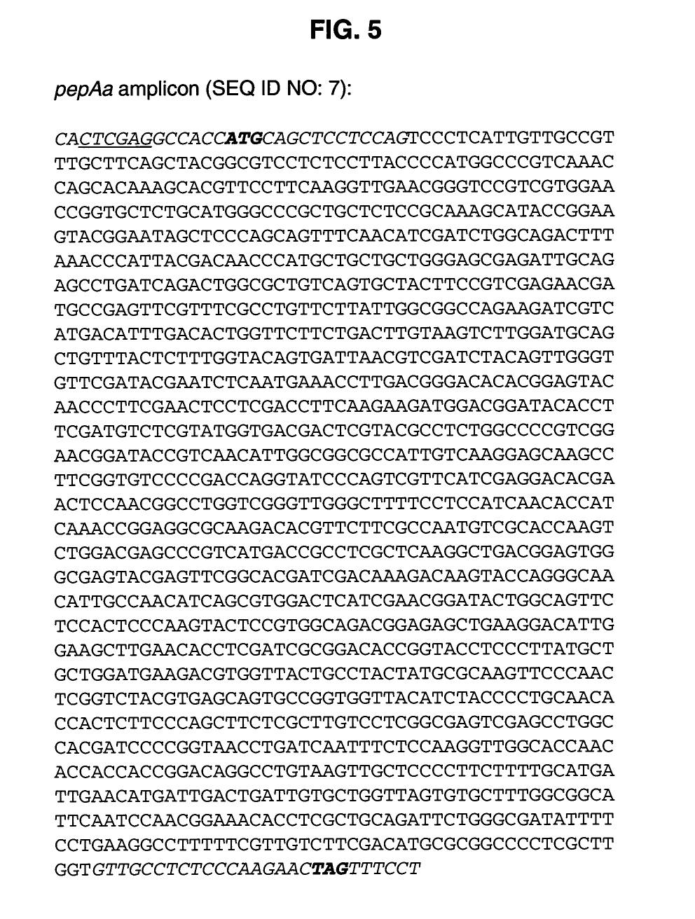

The vector may be any vector which when introduced into a fungal host cell is integrated into the host cell genome and is replicated. Suitable vectors may be found in U.S. Pat. No. 7,332,319, which is hereby incorporated by reference. In one embodiment, the vector used to introduce the pepAx homolog construct into the host cell is the pGAMD vector which comprises the A. niger glucoamylase promoter, a multiple cloning site, and the A. tubingensis glucoamylase terminator, as disclosed in U.S. Pat. No. 7,332,319 (see, pSL898_MunI vector in FIG. 5A). Specific embodiments of pGAMD vectors with pepA homolog gene sequence inserts are described in the following Examples section, including pGAMD-pepAa, pGAMD-pepAa*, pGAMD-pepAb, pGAMD-pepAc, pGAMD-pepAd, pGAMD-pepAd* and pGAMD-pepAd**.

Other suitable vectors are available in the art, as described in, e.g., the Fungal Genetics Stock Center Catalogue of Strains. Additional examples of suitable expression and/or integration vectors are provided in Sambrook et al. (1989) supra, Ausubel (1987) supra, van den Hondel et al. (1991) in Bennett and Lasure (Eds.) MORE GENE MANIPULATIONS IN FUNGI, Academic Press pp. 396-428 and U.S. Pat. No. 5,874,276. Particularly useful vectors include pFB6, pBR322, PUC18, pUC100 and pENTR/D.

In some embodiments, nucleic acid encoding a PEPAx is operably linked to a suitable promoter, which shows transcriptional activity in the filamentous fungal host cell. The promoter may be derived from genes encoding proteins either homologous or heterologous to the host cell. In some embodiments, the promoter is useful in a Trichoderma host or Aspergillus host. Suitable nonlimiting examples of promoters include cbh1, cbh2, egl1, egl2, hfb1, hfb2, xyn1, spt1, pepA, glaA, and amyA.

In some embodiments, the promoter is one that is native to the host cell. For example, when T. reesei is the host, the promoter is a native T. reesei promoter. In a preferred embodiment, the promoter is T. reesei cbh1, which is an inducible promoter and has been deposited in GenBank under Accession No. D86235. In another embodiment, the promoter is one that is heterologous to the fungal host cell.

Other promoters useful with the present compositions and methods include, but are not limited to, those for the following genes: A. awamori and A. niger glucoamylase (glaA) (see, e.g., Nunberg et al. Mol. Cell. Biol. 4:2306-15 (1984); U.S. Pat. Nos. 5,364,770 and 6,590,078 (see, e.g., Example 3), each of which is hereby incorporated by reference herein; Gwynne D. et al. BioTechnol. 5:713-10 (1987); and Boel et al. EMBO J. 3:1581-85 (1984)); Aspergillus niger alpha amylases, Aspergillus oryzae TAKA amylase, T. reesei xln1, T. reesei cellobiohydrolase 1 (see, e.g., EP 0 137 280 A1, which is hereby incorporated by reference), Rhizomucor miehei aspartic proteinase and A. niger neutral alpha amylase.

In some embodiments, the pepA homolog gene coding sequence is operably linked to a signal sequence. The DNA encoding the signal sequence is preferably that which is naturally associated with the particular pepA homolog gene to be expressed.

In some embodiments, the expression vector also includes a termination sequence (i.e., a terminator). The terminator can be native to the host cell or from a different source.

In one embodiment, the vector comprises a terminator and a promoter derived from different sources. In another embodiment, the terminator is homologous to the host cell. Filamentous fungal cell terminators useful with the present include those for the glucoamylase genes from A. niger, A. tubingensis, or A. awamori (see, e.g., Nunberg et al. Mol. Cell. Biol. 4:2306-15 (1984) and Boel et al. EMBO J. 3:1581-85 (1984)).

In some embodiments, the vector can include a selectable marker. Examples of selectable markers include but are not limited to ones that confer antimicrobial resistance (see, e.g., hygromycin, bleomycin, chloroamphenicol and phleomycin). Genes that confer metabolic advantage, such as nutritional selective markers, also find use in the present compositions and methods, including those markers known in the art as amdS, argB and pyr4. Markers useful in vector systems for transformation of Trichoderma and Aspergillus are known in the art (see, e.g., Finkelstein et al. in Chapter 6 of BIOTECHNOLOGY OF FILAMENTOUS FUNGI, Eds. Butterworth-Heinemann, Boston, Mass. (1992); and Kinghorn et al. (1992) APPLIED MOLECULAR GENETICS OF FILAMENTOUS FUNGI, Blackie Academic and Professional, Chapman and Hall, London).

In some embodiments, the selective marker is the amdS gene, which encodes the enzyme acetamidase, allowing transformed cells to grow on acetamide as a nitrogen source. The use of A. nidulans amdS gene as a selective marker (see, e.g., Kelley et al. EMBO J. 4:475-79 (1985); and Penttila et al. Gene 61:155-64 (1987)). In some embodiments, the vector will include the A. niger pyrG gene as a selectable marker and the Aspergillus strain that is transformed using a pyrG marker will be a pyrG mutant strain.

An expression vector comprising a DNA construct with a polynucleotide encoding an aspartic protease may be any vector which is capable of replicating autonomously in a given fungal host organism or of integrating into the DNA of the host. In some embodiments, the expression vector is a plasmid. In preferred embodiments, two types of expression vectors for obtaining expression of genes are contemplated. The first expression vector comprises DNA sequences in which a promoter, a pepA homolog coding region, and a terminator all originate from the gene to be expressed. In some embodiments, gene truncation is obtained by deleting undesired DNA sequences (e.g., DNA encoding unwanted domains) to leave the domain to be expressed under control of its own transcriptional and translational regulatory sequences. The second type of expression vector is preassembled and contains sequences required for high-level transcription and a selectable marker.

In some embodiments, the coding region for an pepA homolog gene or part thereof is inserted into this general-purpose expression vector such that it is under the transcriptional control of the expression construct promoter and terminator sequences.

Methods used to ligate the DNA construct comprising a polynucleotide encoding an pepA homolog, a promoter, a terminator and other sequences and to insert them into a suitable vector are well known in the art. Linking is generally accomplished by ligation at convenient restriction sites. If such sites do not exist, the synthetic oligonucleotide linkers are used in accordance with conventional practice (see, e.g., Sambrook (1989) supra, and Bennett and Lasure, MORE GENE MANIPULATIONS IN FUNGI, Academic Press, San Diego (1991) pp 70-76.). Additionally, vectors can be constructed using known recombination techniques (e.g., Invitrogen Life Technologies, Gateway Technology).

Promoters and Terminators for Recombinant pepA Homolog Expression

In one embodiment, the nucleic acid encoding the pepA homolog will be operably linked to a suitable promoter, which shows transcriptional activity in a fungal host cell. The promoter may be derived from genes encoding proteins either endogenous or heterologous to the host cell. The promoter may be a truncated or hybrid promoter. Further the promoter may be an inducible promoter. Typically, the promoter is useful in a Trichoderma host. Suitable nonlimiting examples of promoters include cbh1, cbh2, egl1, egl2, stp1, and xyn1 (see, e.g., EP 0 137 280 A1, which is hereby incorporated by reference).

In one embodiment, the promoter is one that is native to the host cell. Other examples of useful promoters include promoters from the genes of A. awamori and A. niger glucoamylase genes (glaA) (Nunberg et al. (1984) Mol. Cell. Biol. 4:2306-15 and Boel et al. (1984) EMBO J. 3:1581-85); Aspergillus oryzae TAKA amylase; Aspergillus niger neutral alpha-amylase; Aspergillus niger acid stable alpha-amylase and mutant, truncated and hybrid promoters thereof.

In some embodiments, the polypeptide coding sequence is operably linked to a signal sequence which directs the encoded polypeptide into the cell's secretory pathway. The 5′ end of the coding sequence may naturally contain a signal sequence naturally linked in translation reading frame with the segment of the coding region which encodes the secreted polypeptide. The DNA encoding the signal sequence typically is the sequence which is naturally associated with the polypeptide to be expressed. Typically, the signal sequence is encoded by an Aspergillus niger alpha-amylase, Aspergillus niger neutral amylase or Aspergillus niger glucoamylase. In some embodiments, the signal sequence is the Trichoderma cdh1 signal sequence which is operably linked to a cdh1 promoter.

Transformation of Filamentous Fungal Cells

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; agrobacterium mediated transformation and protoplast fusion. General transformation techniques are known in the art (see, e.g., Ausubel et al. (1987), supra, chapter 9; and Sambrook (1989) supra, Campbell et al. (1989) Curr. Genet. 16:53-56 and THE BIOTECHNOLOGY OF FILAMENTOUS FUNGI, Chap. 6. Eds. Finkelstein and Ball (1992) Butterworth and Heinenmann, each of which is hereby incorporated by reference).

Production of heterologous proteins in filamentous fungal cell expression systems are also known in the art. For example, the expression of heterologous proteins in Trichoderma is described in Harkki et al. (1991) Enzyme Microb. Technol. 13:227-33; Harkki et al. (1989) Bio Technol. 7:596-603; EP 244,234; EP 215,594; and Nevalainen et al. “The Molecular Biology of Trichoderma and its Application to the Expression of Both Homologous and Heterologous Genes”, in MOLECULAR INDUSTRIAL MYCOLOGY, (eds.) Leong and Berka, Marcel Dekker Inc., NY (1992) pp. 129-48; and U.S. Pat. Nos. 6,022,725 and 6,268,328, each of which is hereby incorporated by reference.

The expression of heterologous proteins in Aspergillus spp. is described in Cao et al. (2000) Science 9:991-1001; and U.S. Pat. No. 6,509,171, each of which is hereby incorporated by reference.

In some embodiments, genetically stable transformants are constructed with vector systems, wherein the nucleic acid encoding an aspartic protease (e.g., a PEPA homolog having at least 90% identity to sequence selected from the group consisting of SEQ ID NOs: 1-4, and 21) is integrated into the host strain chromosome. Transformants may then purified by known techniques.

In one nonlimiting example, stable transformants including an amdS marker are distinguished from unstable transformants by their faster growth rate and the formation of circular colonies with a smooth, rather than ragged outline on solid culture medium containing acetamide. Additionally, in some cases a further test of stability may be conducted by growing the transformants on solid non-selective medium (i.e., medium that lacks acetamide), harvesting spores from this culture medium and determining the percentage of these spores which subsequently germinate and grow on selective medium containing acetamide. Alternatively, other methods known in the art may be used to select transformants.

In one embodiment, the preparation of Trichoderma spp. or Aspergillus spp. for transformation involves the preparation of protoplasts from fungal mycelia (see, e.g., Campbell et al. (1989) Curr. Genet. 16:53-56). In some embodiments, the mycelia are obtained from germinated vegetative spores. The mycelia are treated with an enzyme that digests the cell wall resulting in protoplasts. The protoplasts are then protected by the presence of an osmotic stabilizer in the suspending medium. These stabilizers include sorbitol, mannitol, potassium chloride, magnesium sulfate and the like. Usually the concentration of these stabilizers varies between about 0.8 M and about 1.2 M. In some embodiments, it may be preferable to use sorbitol and in other embodiments it may be preferable to use magnesium sulfate (e.g., about a 1.2 M solution of sorbitol in the suspension medium or about a 0.8 M magnesium sulfate solution in the suspension medium).

Uptake of DNA into the host strain may be dependent upon the calcium ion concentration. Generally, between about 10 mM CaCl₂ and 50 mM CaCl₂ may be used in an uptake solution. Besides the need for the calcium ion in the uptake solution, other compounds generally included are a buffering system such as TE buffer (10 mM Tris, pH 7.4; 1 mM EDTA) or 10 mM MOPS, pH 6.0 buffer (morpholinepropanesulfonic acid) and polyethylene glycol (PEG).

Usually, a suspension containing the host cells or protoplasts, such as Trichoderma sp. or Aspergillus sp. protoplasts or cells, which have been subjected to a permeability treatment at a density of about 10⁵ to about 10⁷ per ml, preferably about 2×10⁶ per ml, and also about 1×10⁷ are used in transformations. In some embodiments, a volume of about 100 μL of these protoplasts or cells in an appropriate solution (e.g., 1.2 M sorbitol; 50 mM CaCl₂) are mixed with the desired DNA. In some embodiments, a high concentration of PEG is added to the uptake solution. From 0.1 to 1 volume of 25% PEG 4000 can be added to the protoplast suspension. However, it is preferable to add about 0.25 volumes to the protoplast suspension. Additives such as dimethyl sulfoxide, heparin, spermidine, potassium chloride and the like may also be added to the uptake solution and aid in transformation. Similar procedures are available for other fungal host cells (see, e.g., U.S. Pat. Nos. 6,022,725 and 6,268,328, both of which are hereby incorporated by reference).

Generally, the mixture is then incubated at approximately 0° C. for a period of between 10 to 30 minutes. Additional PEG may be added to the mixture to further enhance the uptake of the desired gene or DNA sequence. In some embodiments, a 25% PEG 4000 is added in volumes of 5 to 15 times the volume of the transformation mixture; however, greater and lesser volumes may be suitable. The 25% PEG 4000 is preferably about 10 times the volume of the transformation mixture. After the PEG is added, the transformation mixture may be incubated either at room temperature or on ice before the addition of a sorbitol and CaCl₂ solution. The protoplast suspension is then further added to molten aliquots of a growth medium. When the growth medium includes a growth selection (e.g., acetamide or an antibiotic) it permits the growth of transformants only.

Cell Culture

The filamentous fungal cells may be grown in conventional culture medium. The culture media for transformed cells may be modified as appropriate for activating promoters and selecting transformants. The specific culture conditions, such as temperature, pH and the like will be apparent to those skilled in the art.

Generally, cells can be cultured in a standard medium containing physiological salts and nutrients (see, e.g., Pourquie, J. et al. BIOCHEMISTRY AND GENETICS OF CELLULOSE DEGRADATION, eds. Aubert, J. P. et al., Academic Press, pp. 71-86, 1988 and Ilmen, M. et al. (1997) Appl. Environ. Microbiol. 63:1298-1306). Common commercially prepared media (e.g., Yeast Malt Extract (YM) broth, Luria Bertani (LB) broth and Sabouraud Dextrose (SD) broth) also find use in the present compositions and methods.

Culture conditions are also standard, (e.g., cultures are incubated at approximately 28° C. in appropriate medium in shake cultures or fermenters until desired levels of PEPA homolog expression are achieved). Culture conditions for a given filamentous fungus are known in the art and may be found in the scientific literature and/or from the source of the fungi such as the American Type Culture Collection and Fungal Genetics Stock Center.

After fungal growth has been established, the cells are exposed to conditions effective to cause or permit the expression and secretion of a PEPA homolog as defined herein. In cases where the pepA homolog sequence is under the control of an inducible promoter, the inducing agent is added to the medium at a concentration effective to induce PEPA homolog expression.

Typical culture conditions for filamentous fungi useful with the present compositions and methods are well known and may be found in the scientific literature such as Sambrook (1982) supra, and from the American Type Culture Collection. Additionally, fermentation procedures for production of heterologous proteins are known per se in the art. For example, proteins can be produced either by solid or submerged culture, including batch, fed-batch and continuous-flow processes. Fermentation temperature can vary somewhat, but for filamentous fungi such as Aspergillus niger the temperature generally will be within the range of about 20° C. to 40° C., typically in the range of about 28° C. to 37° C., depending on the strain of microorganism chosen. The pH range in the aqueous microbial ferment (fermentation admixture) should be in the exemplary range of about 2.0 to 8.0. With filamentous fungi, the pH normally is within the range of about 2.5 to 8.0; with Aspergillus niger the pH normally is within the range of about 4.0 to 6.0, and typically in the range of about 4.5 to 5.5. While the average retention time of the fermentation admixture in the fermentor can vary considerably, depending in part on the fermentation temperature and culture employed, generally it will be within the range of about 24 to 500 hours, typically about 24 to 400 hours. Any type of fermentor useful for culturing filamentous fungi may be employed in the present compositions and methods. One useful embodiment of the present compositions and methods is operation under 15 L Biolafitte (Saint-Germain-en-Laye, France).

### Fermentation

In some embodiments of the present compositions and methods, filamentous fungal cells expressing a recombinant aspartic protease are grown under batch or continuous fermentation conditions. A classical batch fermentation is a closed system, wherein the composition of the medium is set at the beginning of the fermentation and is not subject to artificial alterations during the fermentation. Thus, at the beginning of the fermentation the medium is inoculated with the desired organism(s). In this method, fermentation is permitted to occur without the addition of any components to the system. Typically, a batch fermentation qualifies as a “batch” with respect to the addition of the carbon source and attempts are often made at controlling factors such as pH and oxygen concentration. The metabolite and biomass compositions of the batch system change constantly up to the time the fermentation is stopped. Within batch cultures, cells progress through a static lag phase to a high growth log phase and finally to a stationary phase where growth rate is diminished or halted. If untreated, cells in the stationary phase eventually die. In general, cells in log phase are responsible for the bulk of production of end product.

A variation on the standard batch system is the “fed-batch fermentation” system, which also finds use with the present compositions and methods. In this variation of a typical batch system, the substrate is added in increments as the fermentation progresses. Fed-batch systems are useful when catabolite repression is apt to inhibit the metabolism of the cells and where it is desirable to have limited amounts of substrate in the medium. Measurement of the actual substrate concentration in fed-batch systems is difficult and is therefore estimated on the basis of the changes of measurable factors such as pH, dissolved oxygen and the partial pressure of waste gases such as CO₂. Batch and fed-batch fermentations are common and well known in the art.

Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth and/or end product concentration. For example, in one embodiment, a limiting nutrient such as the carbon source or nitrogen source is maintained at a fixed rate an all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off must be balanced against the cell growth rate in the fermentation. Methods of modulating nutrients and growth factors for continuous fermentation processes as well as techniques for maximizing the rate of product formation are well known in the art of industrial microbiology.

Signal Peptides and Secretion of PEPAx Proteins

The present compositions and methods provide filamentous fungal cells that secrete a recombinant polypeptide encoded by a pepA homolog in an amount at least about 10%, at least about 25%, at least about 50%, at least about 100%, at least about 250%, at least about 500%, at least about 1000%, or even greater than the amount secreted by the corresponding parent strain. In some embodiments, the amount of secreted polypeptide can be determined by comparison of SDS-PAGE gel images of cultured supernatant obtained from the host cell and a cell of the corresponding parent strain.

Secretion of polypeptide by a filamentous fungal cell is typically associated with the presence of a signal peptide at the N-terminal end of the polypeptide. PEPA homolog proteins, PEPAa (SEQ ID NO: 1), PEPAc (SEQ ID NO: 3) and PEPAd (SEQ ID NO: 4) appear to contain a signal peptide comprising the N-terminal amino acids 1-19, 1-20, and 1-19, respectively. Possible KexB cleavage sites (Arginine and Lysine) were detected in PEPAa, PEPAc and PEPAd. A signal peptide sequence could not be identified for PEPAb. The absence of a ‘typical’ signal peptide sequence in a secreted protein, however, is not unprecedented. For example, the superoxide dismutase BcSOD1 in B. cinerea also lacks a signal peptide sequence but was unequivocally demonstrated to be a secreted protein (Rolke et al., “Functional analysis of H₂O₂-generating systems in Botrytis cinerea: the major Cu—Zn-superoxide dismutase (BcSOD1) contributes to virulence on French bean, whereas a glucose oxidase (BcGOD1) is dispensable,” Mol. Plant. Pathol. 5, 17-28 (2004)).

Recombinant Enzyme Recovery

Once the desired recombinant PEPAx enzyme is expressed and secreted by the host filamentous fungal it may be recovered and further purified. The recovery and purification of the protein of interest from a fermentation broth can be done by procedures known in the art. The fermentation broth will generally contain cellular debris, including cells, various suspended solids, and other biomass contaminants, as well as the desired protein product.

Suitable processes for such removal include conventional solid-liquid separation techniques such as, e.g., centrifugation, filtration, dialysis, microfiltration, rotary vacuum filtration, or other known processes, to produce a cell-free filtrate. Often, it may be useful to further concentrate the fermentation broth or the cell-free filtrate prior to crystallization using techniques such as ultrafiltration, evaporation or precipitation.

Precipitating the proteinaceous components of the supernatant or filtrate may be accomplished by means of a salt, followed by purification by a variety of chromatographic procedures, e.g., ion exchange chromatography, affinity chromatography or similar art recognized procedures. When the expressed desired polypeptide is secreted the polypeptide may be purified from the growth media. Typically, the expression host cells are removed from the media before purification of the polypeptide (e.g., by centrifugation).

When the expressed recombinant desired polypeptide is not secreted from the host cell, usually the host cell is disrupted and the polypeptide released into an aqueous “extract” which is the first stage of purification. Typically, the expression host cells are collected from the media before the cell disruption (e.g., by centrifugation).

Recombinant Expression of Enzymes by Host Cells

In some embodiments of the compositions and methods, a filamentous fungal cell is genetically engineered to express a recombinant aspartic protease having at least 80%, 81%, 82%, 83%, 84%, 85%, 86%, 87%, 88%, 89%, 90%, 91%, 92%, 93%, 94%, 95%, 96%, 97%, 98%, 99%, or higher, amino acid sequence identity with any of SEQ ID NOs: 1-4 and 21.

The gene encoding PepA aspartic protease is highly expressed in many strains of Aspergillus niger. In some embodiments, the Aspergillus host strain includes an inactivated pepA aspartic protease gene. Numerous Aspergillus strains can be engineered with pepA or other protease genes inactivated.

In other embodiments, the compositions and methods comprise a nucleotide sequence which encodes a polypeptide having an amino acid sequence of SEQ ID NOs: 1-4, or a truncated version of any one of these polypeptides having aspartic protease activity.

In one embodiment, the polynucleotide encodes a truncated enzyme having amino acid sequence of SEQ ID NO: 21.

In some embodiments, the filamentous fungal cell comprising an inactivated pepA gene and a recombinant gene comprising a pepA homolog has improved properties compared to corresponding parent strains that do not include the inactivated pepA gene and/or the recombinant pepA homolog gene. These improved properties may include for example, increased secreted aspartic protease activity, increased aspartic protease stability at lower pH levels or increased specific activity.

In some embodiments, a recombinant PEPAx aspartic protease (e.g., PEPAa, PEPAa*, PEPAb, PEPAc, PEPAd, PEPAd*, PEPAd**) produced by a filamentous fungal cell may exhibit greater activity at lower pH than corresponding PEPAx endogenously produced from a native host under essentially the same conditions. Thus, in some embodiments, the level of enzyme activity and/or enzyme stability will be at least 0.5, 1.0, 2.0, or 2.5 times greater at a specific pH level compared to an endogenously expressed PEPAx at the same pH.

In some embodiments, a recombinant PEPAx aspartic protease (e.g., PEPAa, PEPAa*, PEPAb, PEPAc, PEPAd, PepAd*, PEPAd**) produced by a filamentous fungal cell may exhibit greater activity at higher temperature than corresponding PEPAx endogenously produced from a native host under essentially the same conditions. Thus, in some embodiments, the level of enzyme activity and/or enzyme stability will be at least 0.5, 1.0, 2.0, or 2.5 times greater at a specific temperature compared to an endogenously expressed PEPAx at the same temperature.

Detection and Measurement of Enzyme activity

In order to evaluate the expression of an aspartic protease (e.g., a PEPA homolog having aspartic protease activity) by a cell line that has been transformed with a heterologous polynucleotide encoding an aspartic protease encompassed by the compositions and methods, assays can be carried out at the protein level, the RNA level or by use of functional bioassays particular to aspartic protease activity and/or production. In general assays employed include, Northern blotting, dot blotting (DNA or RNA analysis), RT-PCR (reverse transcriptase polymerase chain reaction), or in situ hybridization, using an appropriately labeled probe (based on the nucleic acid coding sequence) and conventional Southern blotting and autoradiography.

Various assays are known to those of ordinary skill in the art for detecting and measuring activity of intracellularly and extracellularly expressed polypeptides. Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS). However, other methods are known to those in the art and find use in assessing the protein of interest (see, e.g., Hampton et al., “SEROLOGICAL METHODS, A LABORATORY MANUAL,” APS Press, St. Paul, Minn. (1990) and Maddox et al. J. Exp. Med. 158:1211 (1983), each of which is hereby incorporated by reference).

In addition, the production and/or expression of an aspartic protease enzyme encompassed by the compositions and methods may be measured in a sample directly, for example, by assays directly measuring proteolysis in the culture media and by assays for measuring low pH proteolytic activity, expression and/or production. Substrates useful for assaying aspartic protease activity include casein.

In addition, gene expression may be evaluated by immunological methods, such as immunohistochemical staining of cells, tissue sections or immunoassay of tissue culture medium, e.g., by Western blot or ELISA. Such immunoassays can be used to qualitatively and quantitatively evaluate expression of a PEPA homolog. The details of such methods are known to those of skill in the art and many reagents for practicing such methods are commercially available.

In some embodiments of the compositions and methods, the PEPA homolog produced by a Trichoderma or Aspergillus host will be greater than 1 gram protein per liter (g/L), greater than 2 g/L, greater than 5 g/L, greater than 10 g/L, greater than 20 g/L, greater than 25 g/L, greater than 30 g/L, greater than 50 g/L and even greater than 100 g/L of culture media.

In some embodiments, the amount of secreted PEPA homolog will be at least about 5%, 10%, 15%, 20%, 25%, 30%, 35%, 40%, 45%, 50%, 55%, 60%, 65%, 70%, 75%, 80%, 95%, or an even greater amount of the total secreted protein from the host strain. In other embodiments, the amount of secreted PEPA homolog will be greater than 50% of the total secreted protein. In some embodiments the amount of secreted PEPA homolog will be less than 50% of the total secreted protein. In some embodiments, the amount of secreted PEPA homolog will be greater than the amount of secreted PEPA homolog from an Aspergillus strain.

Enzyme Purification

In general, an aspartic protease enzyme according to the compositions and methods (such as a PEPA homolog) which is produced in filamentous fungal cell culture is secreted into the medium and may be separated or purified, e.g., by removing unwanted components from the cell culture medium. In some cases, an enzyme may be produced in a cellular form necessitating recovery from a cell lysate. In such cases the enzyme is purified from the cells in which it was produced using techniques routinely employed by those of skill in the art. Examples include, but are not limited to, affinity chromatography (see, e.g., Tilbeurgh et al. (1984) FEBS Lett. 16:215); ion-exchange chromatographic methods (see, e.g., Goyal et al. (1991) Biores. Technol. 36: 37; Fliess et al. (1983) Eur. J. Appl. Microbiol. Biotechnol. 17:314; Bhikhabhai et al. (1984) J. Appl. Biochem. 6:336; and Ellouz et al. (1987) Chromatography 396:307), including ion-exchange using materials with high resolution power (see, e.g., Medve et al. (1998) J. Chromatography A 808:153); hydrophobic interaction chromatography (see, e.g., Tomaz and Queiroz, (1999) J. Chromatography A 865:123); two-phase partitioning (see, e.g., Brumbauer et al. (1999) Bioseparation 7:287); ethanol precipitation; reverse phase HPLC; chromatography on silica or on a cation-exchange resin such as DEAE; chromatofocusing; SDS-PAGE; ammonium sulfate precipitation; and gel filtration using, e.g., Sephadex G-75.

Enzyme Compositions

A particularly useful enzyme composition includes one or more proteases and/or one or more starch hydrolyzing enzymes. Protease include one or more aspartic proteases having at least 80%, 81%, 82%, 83%, 84%, 85%, 86%, 87%, 88%, 89%, 90%, 91%, 92%, 93%, 94%, 95%, 96%, 97%, 98%, 99%, or greater sequence identity to a sequence selected from the group consisting of SEQ ID NOs:1, 2, 3, 4, 21, 23, and 28, or a combination thereof. In some embodiments, the aspartic protease (e.g., PEPA homolog) is obtained from the heterologous expression of a pepA homolog gene, such as the heterologous expression of an Aspergillus kawachi acid stable aspartic protease in a Trichoderma reesei or Aspergillus niger host.

In some embodiments, the compositions and methods encompasses a fermentation or culture medium comprising a PEPA homolog enzyme having acid stable protease activity produced from a culture of Aspergillus cells, said Aspergillus cells comprising a heterologous polynucleotide encoding an PEPA homolog which has at least 80%, 81%, 82%, 83%, 84%, 85%, 86%, 87%, 88%, 89%, 90%, 91%, 92%, 93%, 94%, 95%, 96%, 97%, 98%, 99%, or greater sequence identity to a sequence selected from the group consisting of SEQ ID NOs:1, 2, 3, 4, 21, 23, and 28.

In some embodiments, the enzyme composition comprises a PEPA homolog in a cell free filtrate, i.e., a culture medium isolated from the host cell. In some embodiments, the PEPA homolog is co-expressed into the culture medium along with another enzyme. In other embodiments, the aspartic protease is available in a culture medium containing the fungal host cells which express and secrete the aspartic protease.

In a further aspect, the compositions and methods encompasses a fermentation or culture medium comprising a PEPA homolog produced from a culture of Trichoderma cells, said Trichoderma cells comprising a heterologous polynucleotide encoding the PEPA homolog which has at least 80%, 81%, 82%, 83%, 84%, 85%, 86%, 87%, 88%, 89%, 90%, 91%, 92%, 93%, 94%, 95%, 96%, 97%, 98%, 99%, or greater sequence identity to a sequence selected from the group consisting of SEQ ID NOs:1, 2, 3, 4, 21, 23, and 28.

In another embodiment, the compositions and methods encompass a fermentation or culture medium comprising an acid stable aspartic protease (e.g., PEPA homolog) and an alpha-amylase wherein both the aspartic protease and alpha-amylase are co-expressed from a culture of Aspergillus cells, said cells comprising a heterologous polynucleotide encoding an aspartic protease which has at least 80%, 81%, 82%, 83%, 84%, 85%, 86%, 87%, 88%, 89%, 90%, 91%, 92%, 93%, 94%, 95%, 96%, 97%, 98%, 99%, or greater sequence identity to a sequence selected from the group consisting of SEQ ID NOs:1, 2, 3, 4, and 21 and a polynucleotide coding for the alpha-amylase.

In some embodiments, the aspartic proteases in the culture medium are further recovered. The enzymes may be formulated for use in enzyme compositions for numerous applications. Some of these applications and compositions include but are not limited to for example starch proteolysis and hydrolyzing compositions, cleaning and detergent compositions (e.g., laundry detergents, dish washing detergents, and hard surface cleaning compositions), animal feed compositions, baking applications, such as bread and cake production, brewing applications, healthcare applications, textile applications, environmental waste conversion processes, biopulp processing, and biomass conversion applications.

As understood by those in the art, the quantity of aspartic protease (e.g., PEPA homolog) used in the compositions and methods depends on the enzymatic activity of the aspartic protease. In some embodiments, the range of an aspartic protease encompassed in the enzyme compositions is from 0.001 to 80 SSU, 0.001 to 60 SSU, also 0.01 to 40 SSU; also 0.01 to 30 SSU; also 0.01 to 20 SSU; also 0.01 to 15 SSU; also 0.05 to 15 SSU and also 0.01 to 10 SSU per g ds.

Useful enzyme compositions are enzyme compositions as described above which further comprise a secondary acid stable protease. A secondary acid stable protease is a protease obtained from a source that is different from the Aspergillus host, which comprises the heterologous polynucleotide encoding a PEPA homolog, e.g., a polynucleotide encoding an aspartic protease having at least 80%, 81%, 82%, 83%, 84%, 85%, 86%, 87%, 88%, 89%, 90%, 91%, 92%, 93%, 94%, 95%, 96%, 97%, 98%, 99%, or greater sequence identity to a sequence selected from the group consisting of SEQ ID NOs:1, 2, 3, 4, 21, 23, and 28.

These and other aspects and embodiments of the compositions and methods will be apparent from the description. The manner and method of carrying out the compositions and methods may be more fully understood by those of skill in the art by reference to the following examples, which are not intended to in any manner limit the scope of the present compositions or methods, or of the appended claims directed, thereto.

### EXAMPLES

The following Examples are provided to demonstrate and further illustrate specific embodiments and aspects of the present compositions and methods and are not to be construed as limiting the scope thereof.

In the experimental disclosure which follows, the following abbreviations apply: ° C. (degrees Centigrade); H₂O (water); dH₂O (deionized water); HCl (hydrochloric acid); aa (amino acid); by (base pair); kb (kilobase pair); kD (kilodaltons); g (grams); μg (micrograms); mg (milligrams); μl (microliters); ml (milliliters); mm (millimeters); μm (micrometer); M (molar); mM (millimolar); μM (micromolar); MW (molecular weight); s (seconds); min(s) (minute/minutes); hr(s) (hour/hours); NaCl (sodium chloride); PBS (phosphate buffered saline [150 mM NaCl, 10 mM sodium phosphate buffer, pH 7.2]); PCR (polymerase chain reaction); SDS (sodium dodecyl sulfate); w/v (weight to volume); v/v (volume to volume); ATCC (American Type Culture Collection, Rockville, Md.); BD BioSciences (Previously CLONTECH Laboratories, Palo Alto, Calif.); Invitrogen (Invitrogen Corp., San Diego, Calif.); and Sigma (Sigma Chemical Co., St. Louis, Mo.).

### Example 1

### Construction of a Recombinant pepAa Gene and Production of PEPAa Protein by the Transformed GAP3 Strain of A. niger

The pepAa gene encodes the PEPAa protein of 424 amino acids (SEQ ID NO: 1) with a signal sequence of 19 amino acids. This example illustrates: (1) construction of a plasmid vector, pGAMD-pepAa, having a recombinant gene comprising pepAa inserted between the A. niger glucoamylase promoter and an A. tubingensis glucoamylase terminator; (2) transformation of A. niger with this vector resulting in an integrated recombinant gene; (3) selection A. niger strains that overexpress pepAa; and (4) measurement of production of PEPAa protein by the overexpressing strains.

To construct the recombinant expression plasmid for A. niger pepAa gene, two primers CACTCGAGGCCACCATGCAGCTCCTCCAG (SEQ ID NO: 5) and AGGAAACTAGTTCTTGGGAGAGGCAAC (SEQ ID NO: 6) were used in a Pfu PCR reaction with genomic DNA template obtained from A. niger UVK143 strain (Ward et al. Appl. Microbiol. Biotechnol. 39:738-43 (1993)). The nucleotide sequence of the resulting PCR amplicon (SEQ ID NO: 7) is shown in FIG. 5.

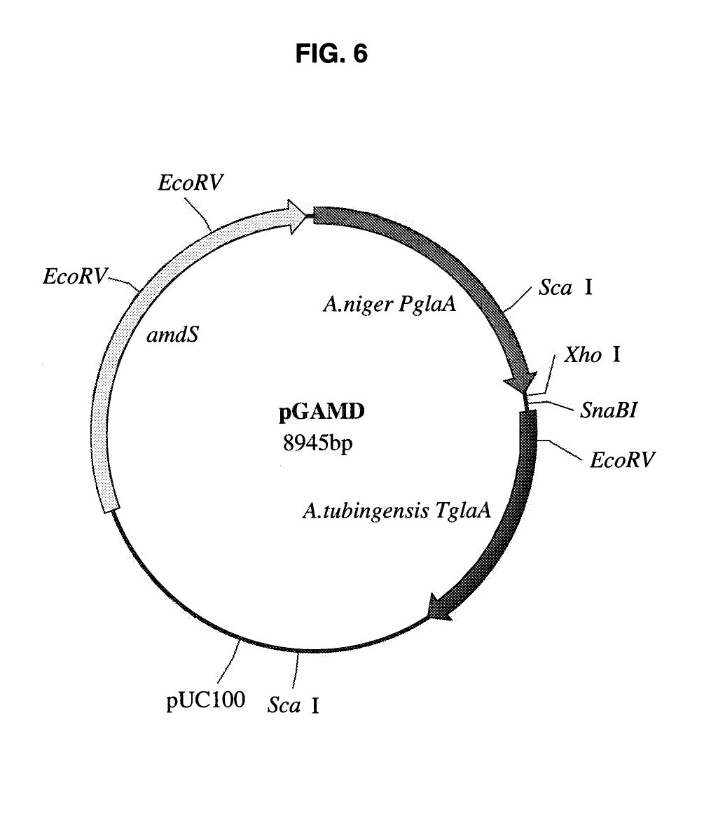

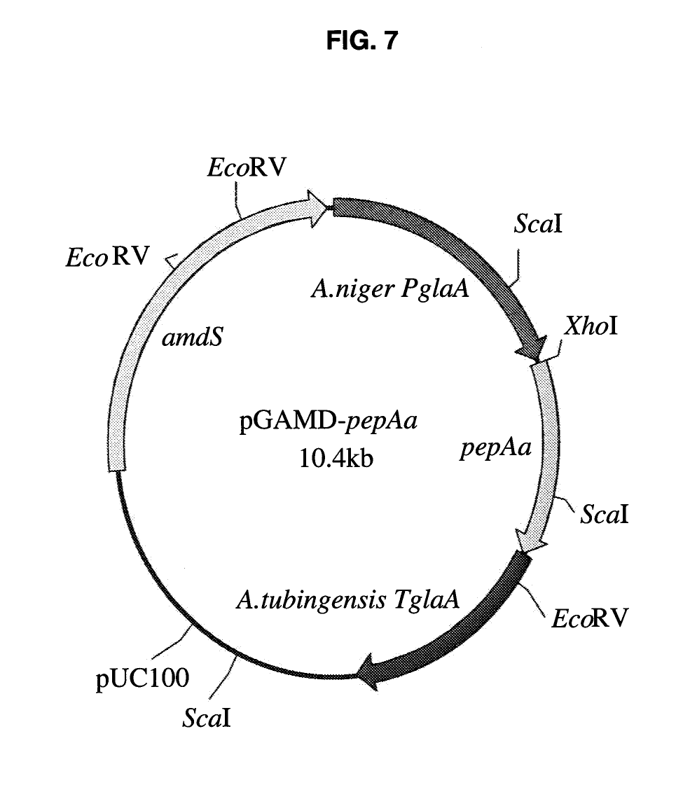

The PCR amplicon (SEQ ID NO: 7) was digested with restriction enzyme XhoI and was cloned into the pGAMD plasmid vector (see, e.g., U.S. Pat. No. 7,332,319, which is hereby incorporated by reference) that had been digested with XhoI and SnaBI. As shown in FIG. 6, the pGAMD plasmid vector has XhoI and SnaBI restriction sites flanked upstream by the A. niger glucoamylase promoter sequence and downstream by the A. tubingensis glucoamylase terminator. The resulting plasmid, pGAMD-pepAa (shown in FIG. 7) was confirmed by DNA sequencing to have a recombinant gene comprising pepAa inserted between the A. niger glucoamylase promoter and an A. tubingensis glucoamylase terminator.

The GAP3 strain of A. niger (Ward et al, Appl. Microbiol. Biotechnol, 39: 738-43, (1993)) was transformed with the pGAMD-pepAa plasmid vector using a PEG-mediated protoplasts fusion transformation protocol. GAP3 strain has an inactivated pepA gene.

The transformation protocol utilized was a modification of the Campbell method (see, Campbell et al., Curr. Genet. 16:53-56 (1989), which is hereby incorporated by reference herein) with beta-D-glucanase G (InterSpex Products, Inc. San Mateo, Calif.) used to produce protoplasts and the pH adjusted to 5.5.

Briefly, protoplast preparation and A. niger transformation were carried out as follows:
(a) a 1-2 ml spore suspension made from fresh slant culture was inoculated into 50 ml liquid medium (soluble starch 3%, yeast extract 2%, KH₂PO₄ 0.5%, corn meal 0.5%, Natural pH), in a shake flask and was cultivated on a rotor shaker at 200 rpm, 30° C. for 13-14 hr;
(b) mycelium was collected by filtrating culture through gauze and washed three times with water, once with 0.8 M MgSO₄ (pH 5.8);
(c) washed mycelium were placed into 100 ml flask, suspended in 15 ml 0.8 M MgSO₄ containing 150 mg of lysing enzyme (Sigma-Aldrich, St. Louis, Mo.) and 15 mg of cellulase R-10 (Yakult Biochemical Co., Ltd., Nishinomiya, Japan);
(d) the mycelium cell wall was digested at 30° C. for 1-2 hrs which flask shaken at 80 rpm, and protoplast formation was monitored under microscope;
(e) protoplasts were harvested from cell lysate by filtering through two layers of 200 mesh nylon membrane to remove cell debris;
(f) protoplasts were collected and washed with sorbitol solution (1.2 M sorbitol, 50 mM CaCl₂, 10 mM Tris pH 7.4) two times by centrifuge at 700g for 6-8 min;
(g) protoplasts were resuspended in 200 μl of sorbitol solution and were counted with a blood counter to determine concentration;
(h) 10 pg transformation vector DNA (pGAMD-pepAa) was mixed with 2−4×10⁷ protoplast;
(i) to the above mixture, 50 μl of PEG6000 (or PEG4000) solution (PEG 50%, 50 mM CaCl₂, 10 mM Tris pH 7.4) were added and mixed gently but thoroughly, and put on ice for 30 min;
(j) 1 ml PEG solution was added, mixed well, and placed at room temperature for 20 min;
(k) 1 ml sorbitol solution was added and mixed well with 56-58° C. molten soft agar and then the whole mixture immediately was poured onto transformation medium plate;
(l) the plate was incubated at 30° C. for 4-8 days.

All solutions and media were either autoclaved or filter sterilized through a 0.2 micron filter.

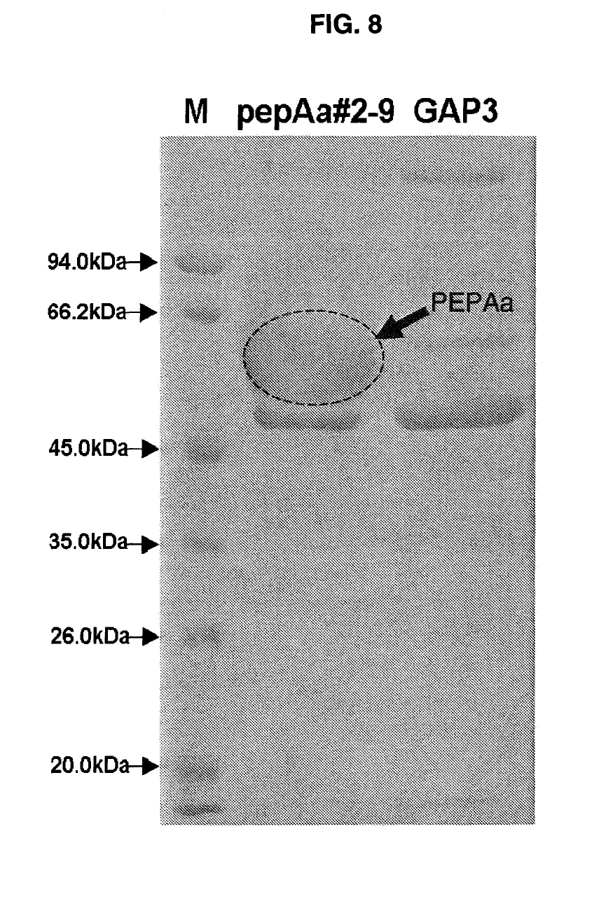

More than sixteen transformants were selected and cultured for 6 days at 28° C., pH 6.2 in 30 ml Promosoy special broth. Culture broths were filtered and supernatant protein content was analyzed using SDS-PAGE analysis. The transformant producing the highest amount of the PEPAa protein was determined to be strain #2. This strain was further spore purified to generate strain #2-9. Production of the PEPAa protein from strain #2-9 was carried out in a shake flask. SDS-PAGE analysis of the #2-9 supernatant was carried out and the PEPAa protein detected at an approximate MW 55 kD as by shown gel image in FIG. 8. The molecular weight of the PEPAa protein (55 kD) on SDS gel is higher than that of the predicted molecular weight (44.9 kD) since the PEPAa protein is glycosylated. Based on the gel image the amount of PEPAa protein produced by strain #2-9 was estimated to be at least about 15% of the total protein secreted by the cell into the supernatant. In contrast, no PEPAa band was detectable in the gel image of the supernatant of the corresponding GAP3 parent strain.

### Example 2

### Construction of a Recombinant Truncated pepAa Gene and Transformation into the GAP3 Strain of A. niger

A recombinant truncated version of pepAa gene (“pepAa*”) was prepared and transformed into GAP3 strain of A. niger to demonstrate the significance of this sequence for secretion of the full-length wild-type PEPAa protein (SEQ ID NO: 1). The full length pepAa gene has a 3′ nucleotide sequence encoding the following C-terminal amino acids beginning at SEQ ID NO: 1 position 382:

LCFGGIQSNGNTSLQILGDIFLKAFFVVFDMRGPSLGVASPKN.

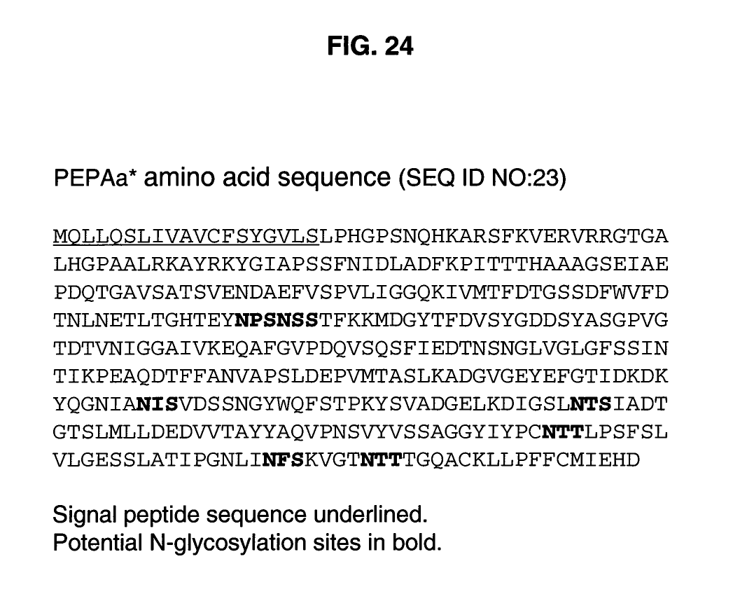

As shown in FIG. 24, the amino acid sequence of the truncated version of the PEPAa* protein (SEQ ID NO: 23) encoded by pepAa*, does not have the C-terminal 43 amino acids of SEQ ID NO: 1, but rather has a different 13 amino acids inserted. Thus, the PEPAa and PEPAa* amino acid sequences from positions 1-381 are identical, but starting at position 382, the PEPAa* sequence (SEQ ID NO: 23) has the C-terminal amino acids:

CKLLPFFCMIEHD.

The methods of preparation used were essentially the same as in Example 1 except as otherwise described below.

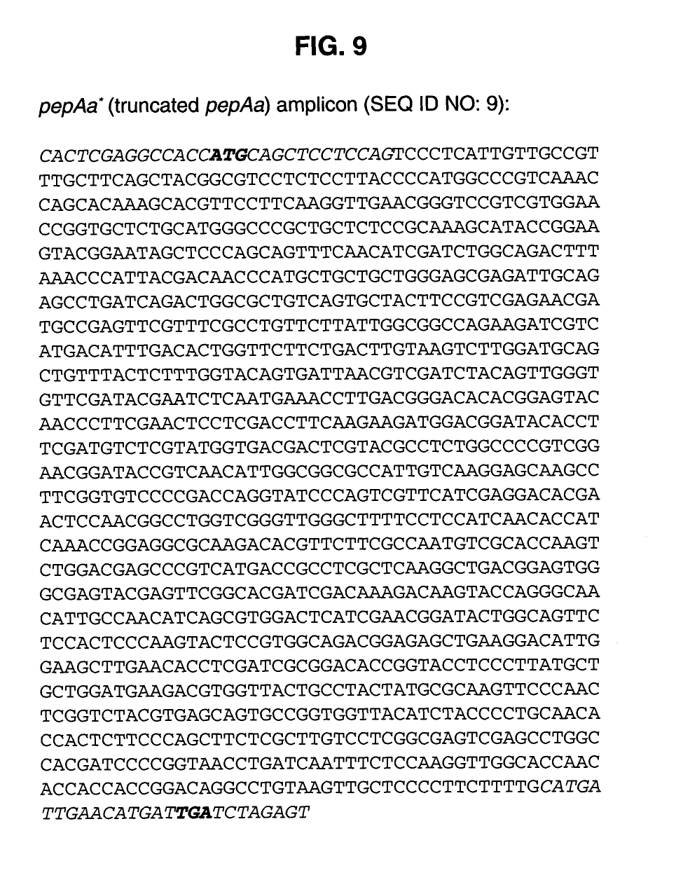

To construct the recombinant expression plasmid for the truncated A. niger pepAa gene (pepAa), the primers CACTCGAGGCCACCATGCAGCTCCTCCAG (SEQ ID NO: 1) and the primer ACTCTAGATCAATCATGTTCAATCATG (SEQ ID NO: 8) were used in the Pfu PCR reaction containing genomic DNA template obtained from the A. niger UVK143 strain. The nucleotide sequence of the resulting PCR amplicon (SEQ ID NO: 9) is shown in FIG. 9.

The PCR amplicon (SEQ ID NO: 9) was digested with restriction enzyme XhoI and was cloned into the pGAMD vector previously digested with XhoI and SnaBI. Fidelity of the resulting plasmid pGAMD-pepAa* was confirmed by DNA sequencing.

Transformation of A. niger GAP3 strain with pGAMD-pepAa* plasmid was carried out as described Example 1. Eight transformants were selected and cultured for 6 days as described in Example 1. SDS-PAGE analysis of the filtered culture broth supernatant to detect secreted protein was carried out as described in Example 1. No PEPAa* was visualized in the gel suggesting that the deleted 43 amino acid sequence plays a critical role in the expression and/or secretion of PEPAa.

### Example 3

Construction of a Recombinant pepAb gene and Production of PEPAb Protein by the Transformed GAP3 Strain of A. niger

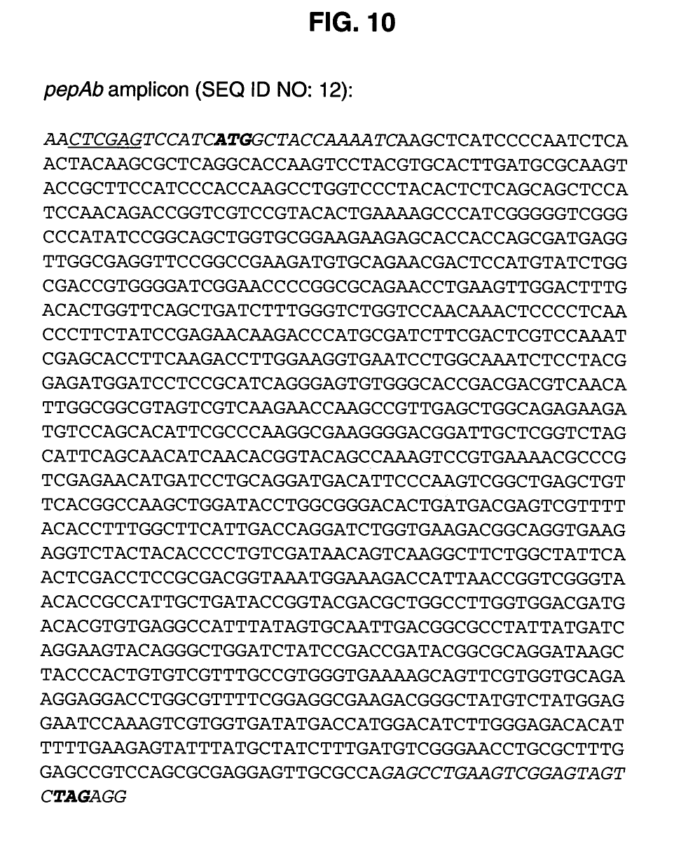

The recombinant expression plasmid for A. niger pepAb gene, was constructed as described for pepAa gene in Example 1 except that the two primers used in the Pfu PCR reaction containing genomic DNA template obtained from UVK143 strain were: AACTCGAGTCCATCATGGCTACCAAAATC (SEQ ID NO: 10) and CCTCTAGACTACTCCGACTTCAGGCTC (SEQ ID NO: 11). The 1418 bp nucleotide sequence of the resulting PCR amplicon (SEQ ID NO: 12) is shown in FIG. 10.

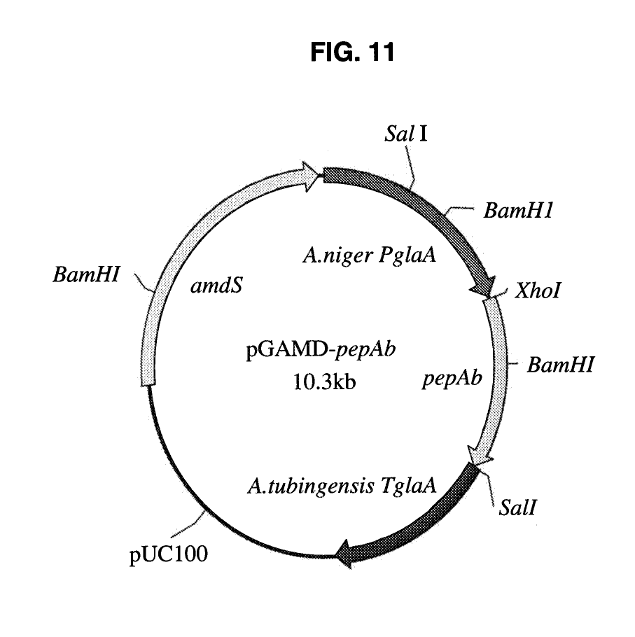

The PCR amplicon (SEQ ID NO: 12) was cloned into the pGAMD vector (as described for the pepAa amplicon in Example 1). The resulting plasmid, pGAMD-pepAb (shown in FIG. 11) was confirmed by DNA sequencing to have a recombinant gene comprising pepAb inserted between the A. niger glucoamylase promoter and an A. tubingensis glucoamylase terminator.

The A. niger GAP3 strain was transformed (as described in Example 1) with the pGAMD-pepAb plasmid vector, eight transformants were selected and cultured, and culture broths supernatants filtered and subjected to SDS-PAGE analysis, as in Example 1.

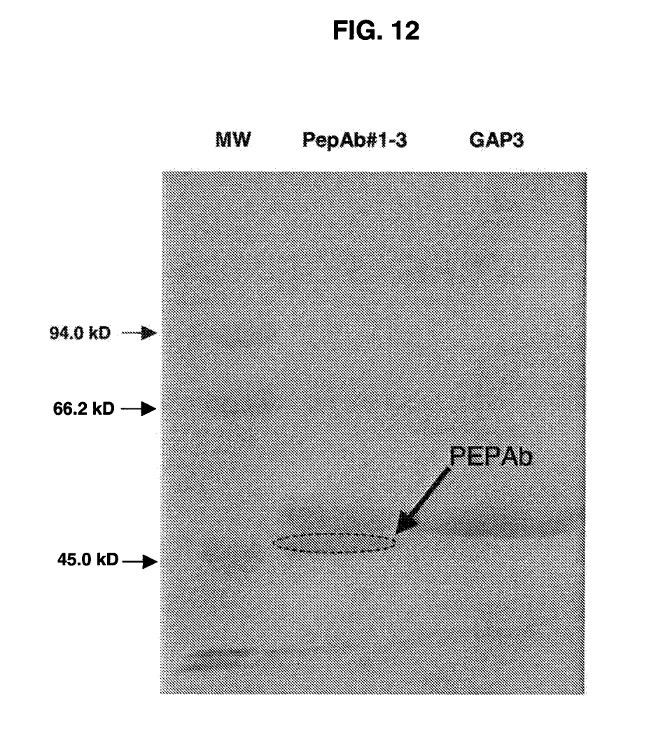

The transformant which produced highest amount of the recombinant pepAb protein (strain #1) was further spore purified to generate the strain #1-3. SDS-PAGE analysis of the GGAb#1-3 supernatant was carried out and the PEPAb protein detected at an approximate MW 47 kD as shown on SDS gel image in FIG. 12. However, mass spectrometric analysis of the GGAb#1-3 supernatant indicated that the PEPAb protein is 38 kD. A smaller 32.7 kD peptide corresponding to the C-terminal portion of PEPAb also detected. Based on the gel image the amount of PEPAb protein produced by #1-3 was estimated to be at least about 10% of the total protein secreted by the cell into the supernatant. In contrast, no PEPAb band was detectable in the gel image of the supernatant of the corresponding GAP3 parent strain.

### Example 4

Construction of a Recombinant pepAc Gene and Production of PEPAc Protein by the Transformed GAP3 Strain of A. niger

The recombinant expression plasmid for A. niger pepAc gene, was constructed as described for pepAa gene in Example 1 except as otherwise described below.

Two pairs of primers were used in the Pfu PCR reaction containing genomic DNA template obtained from UVK143 strain. CACTCGAGTGCCGCCATGTATATCCCCGTC (SEQ ID NO: 13) and TTGCTCAGTTCGAGGTACTTGGAGGAG (SEQ ID NO: 14) were used to amplify DNA fragment encoded for the n-terminus of the protein. AGTACCTCGAACTGAGCAAGACCAAG (SEQ ID NO: 15) and TCTAGTAGGTGTCGTTGGAGGTGTAG (SEQ ID NO: 16) were used to amplify DNA fragment encoded for the c-terminus of the protein.

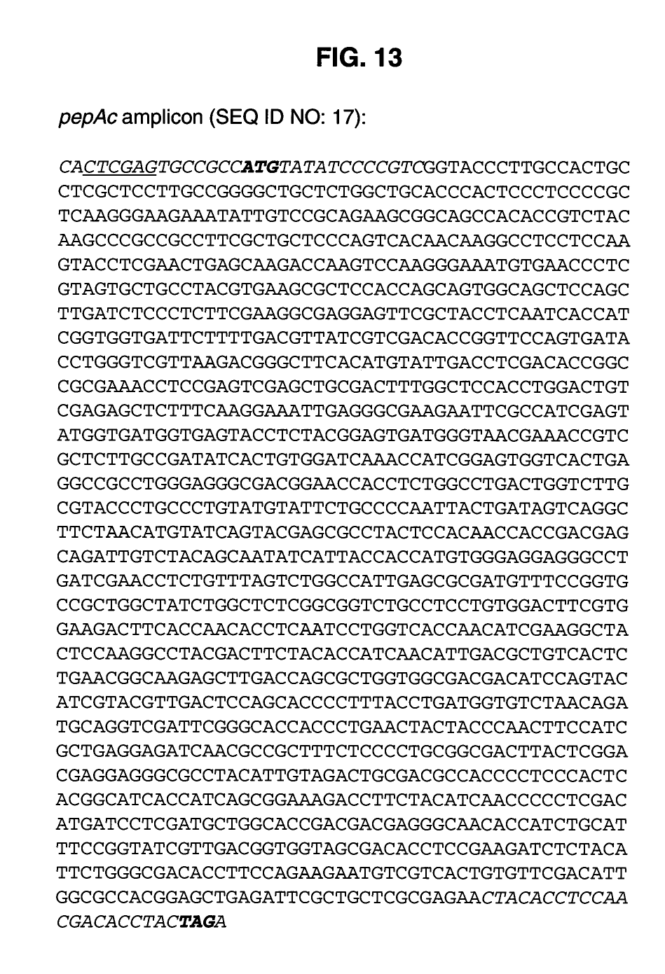

Fusion PCR was used to amplify the pepAc gene fragment with the primers CACTCGAGTGCCGCCATGTATATCCCCGTC (SEQ ID NO: 13) and TCTAGTAGGTGTCGTTGGAGGTGTAG (SEQ ID NO: 16) and the N- and C-terminal PCR amplicon as the DNA template. In the fusion PCR amplicon, the 61st codon of the pepAc gene was changed from GAG to GAC, which removed the internal XhoI restriction site without altering the encoded amino acid sequence. The nucleotide sequence of the resulting PCR amplicon (SEQ ID NO: 17) is shown in FIG. 13.

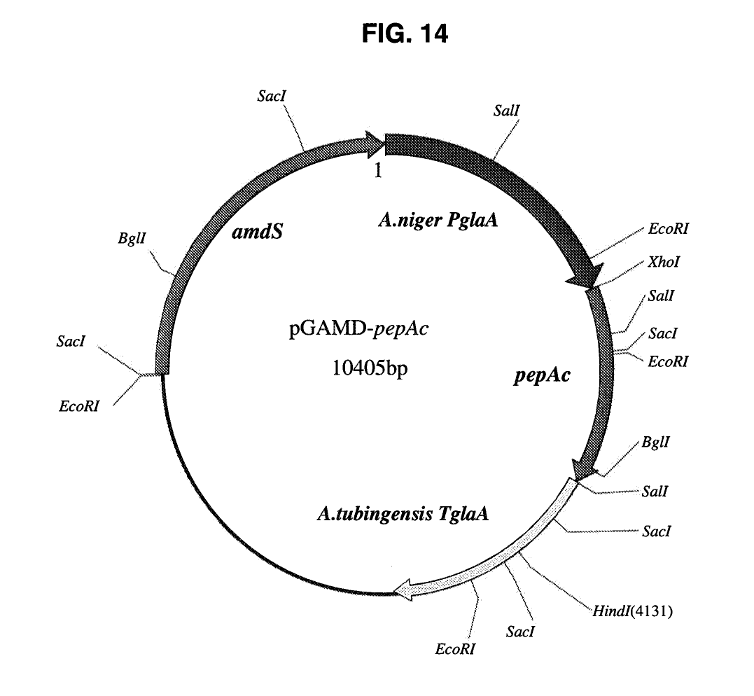

The PCR amplicon (SEQ ID No. 17) was digested with restriction enzyme XhoI and cloned into XhoI and SnaBI digested pGAMD vector. The resulting plasmid, pGAMD-pepAc (shown in FIG. 14) was confirmed by DNA sequencing to have a recombinant gene comprising pepAc inserted between the A. niger glucoamylase promoter and an A. tubingensis glucoamylase terminator.

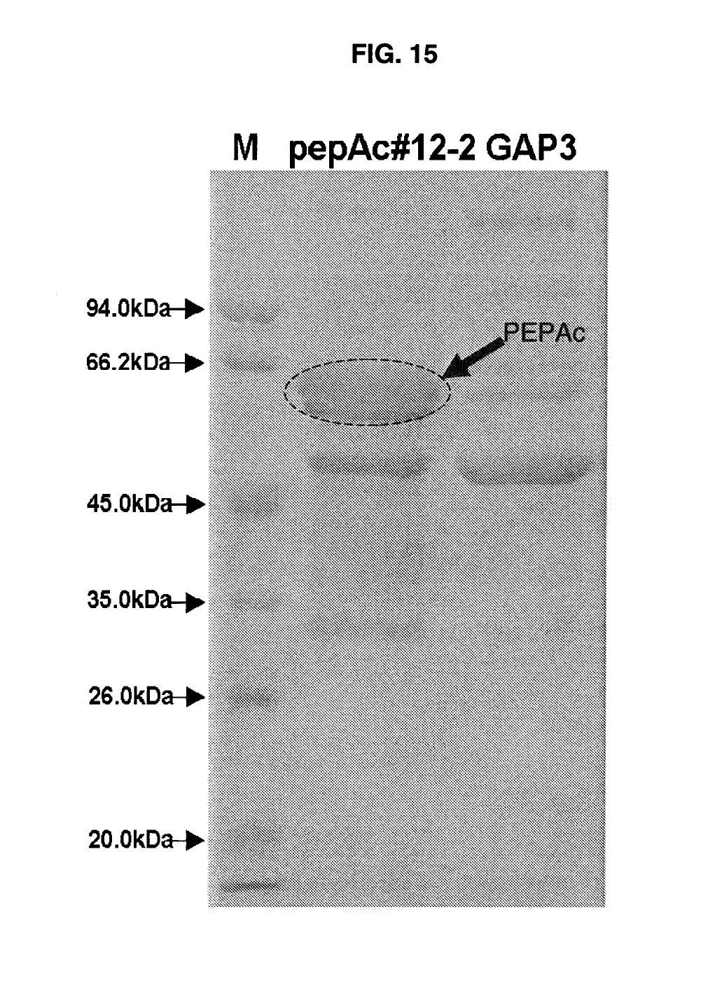

The A. niger GAP3 strain was transformed (as described in Example 1) with the pGAMD-pepAc plasmid vector. Twenty-nine transformants were selected and cultured in 30 ml Promosoy special broth (pH 6.2) for 6 days at 28° C. Culture broths were filtered and supernatant protein content was analyzed using SDS-PAGE. The transformant which produced highest amounts of the PEPAc protein was determined to be strain #12. This strain was further spore purified to generate the strain #12-2, SDS-PAGE analysis of the strain #12-2 supernatant was carried out and the PEPAc protein detected at about 60 kD as shown in FIG. 15. The molecular weight of the PEPAc protein (60 kD) on SDS gel is higher than that of the predicted molecular weight (46.2 kD) since the PEPAc protein is glycosylated. Based on the gel image the amount of PEPAc protein produced by strain #12-2 was estimated to be at least about 10% of the total protein secreted by the cell into the supernatant. In contrast, no PEPAc band was detectable in the gel image of the supernatant of the corresponding GAP3 parent strain.

### Example 5

### Construction of a Recombinant Truncated pepAd Gene (“pepAd”) and Production of Truncated Protein “PepAd” by the Transformed GAP3 Strain of A. niger

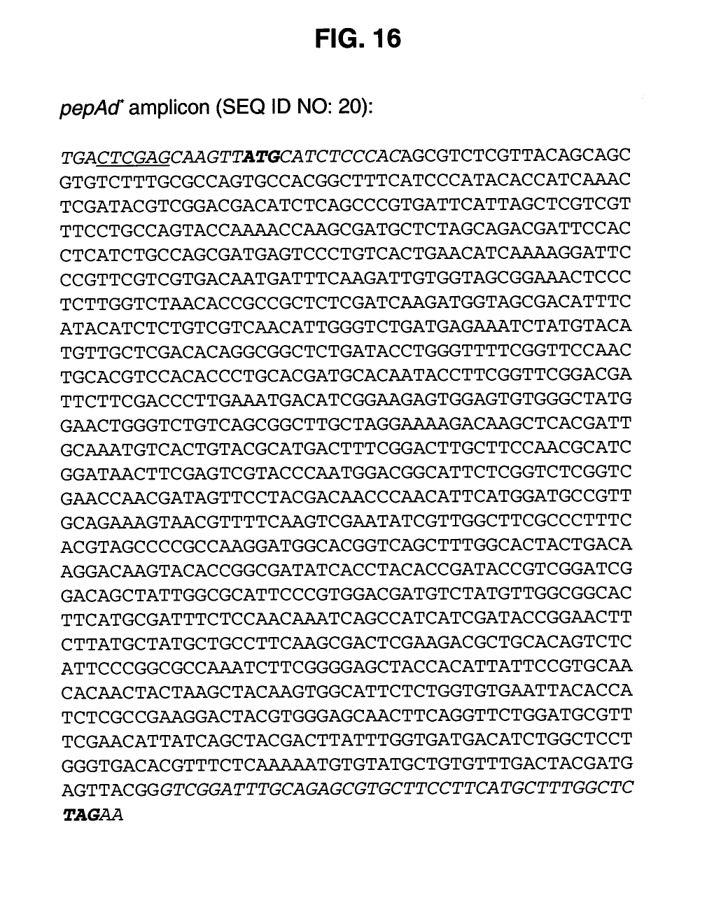

The recombinant expression plasmid for a truncated version of the A. niger pepAd* gene, was constructed as described for pepAa gene in Example 1 except that the two primers used in the Pfu PCR reaction containing genomic DNA template obtained from UVK143 strain were: TGACTCGAGCAAGTTATGCATCTCCCAC (SEQ ID NO: 18) and TTCTAGAGCCAAAGCATGAAGGAAGCACGCTCTGCAAATCCGAC (SEQ ID NO: 19). The nucleotide sequence of the resulting PCR amplicon (SEQ ID NO: 20) is shown in FIG. 16.

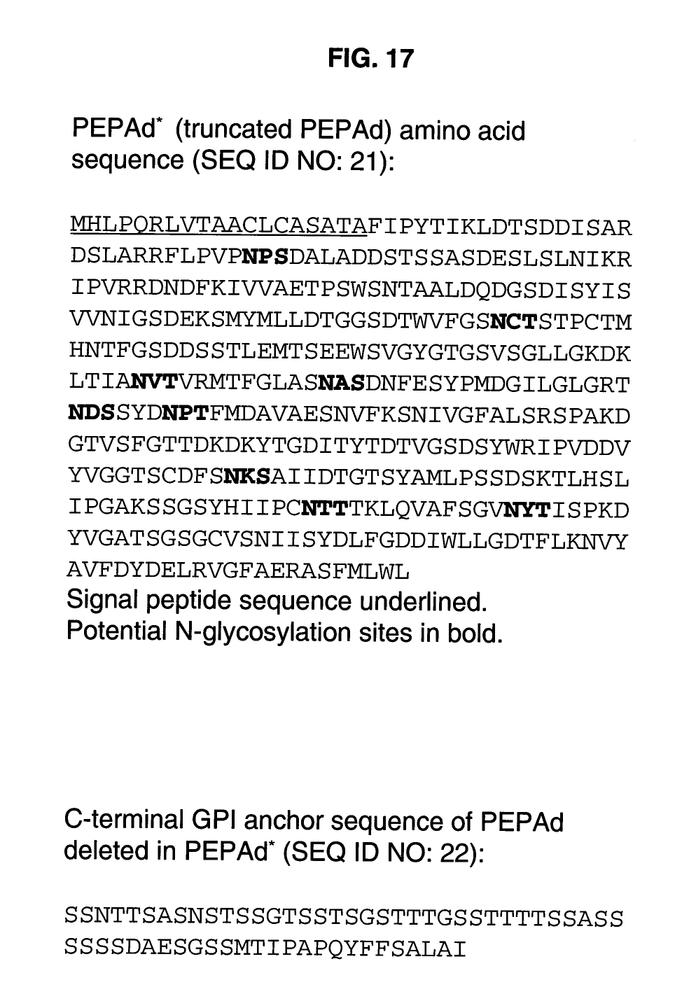

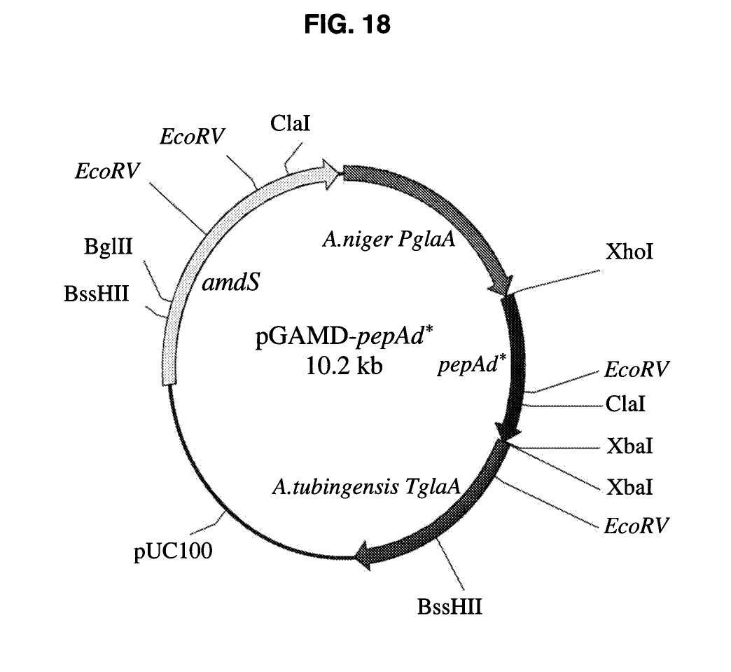

The PCR amplicon (SEQ ID NO: 20) encodes a truncated version of the PEPAd protein, referred to as “PEPAd*”. The amino acid sequence of PEPAd* (SEQ ID NO: 21), shown in FIG. 17, does not include the serine-rich region at the C-terminus (SEQ ID NO: 22) of the native PEPAd sequence (SEQ ID NO: 4), which is predicted to be a GPI anchor sequence. The PCR amplicon (SEQ ID NO: 20) was cloned into the pGAMD vector (as described for pepAa in Example 1). The resulting plasmid vector, pGAMD-pepAd* (shown in FIG. 18) was confirmed by DNA sequencing to have a recombinant gene comprising pepAd* inserted between the A. niger glucoamylase promoter and an A. tubingensis glucoamylase terminator.

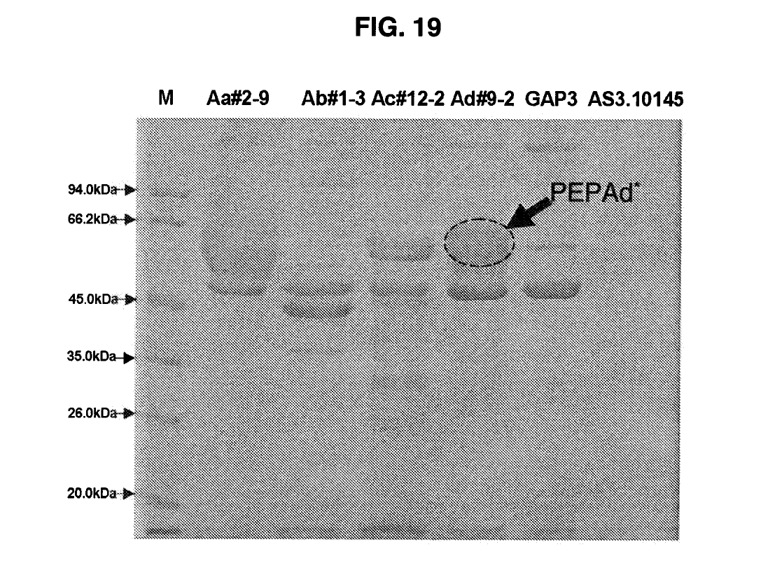

The A. niger GAP3 strain was transformed (as described in Example 1) with the pGAMD-pepAd* plasmid vector, nine transformants were selected and cultured, and culture broths supernatants filtered and subjected to SDS-PAGE analysis, as in Example 1, to determine the strains producing the greatest amount of PEPAd* protein. The transformant which produced highest amount of the recombinant PEPAd* protein (strain #9) was further spore purified to generate strain #9-2. SDS-PAGE analysis of the strain #9-2 supernatant was carried out and the PEPAd* protein detected at about 60 kD. FIG. 19 shows the SDS-PAGE gel image with the PEPAd* band as well as lanes with the spore-purified strains for PEPAa, PEPAb, and PEPAc. The molecular weight of the PEPAd* protein (60 kD) on SDS gel is higher than that of the predicted molecular weight (43 kD) since the PEPAd* protein is glycosylated. The visualization by SDS-PAGE of the overproduction of the PEPAd* protein in the strain #9-2 supernatant indicated that the truncation of the predicted GPI anchor sequence results in a protein capable of secretion into the culture medium. Based on the gel image the amount of PEPAd* protein produced by strain #9-2 was estimated to be at least about 10% of the total protein secreted by the cell into the supernatant. In contrast, no PEPAd band was detectable in the gel image of the supernatant of the corresponding GAP3 parent strain.

### Example 6

### Construction of a Recombinant pepAd Gene and Production of the PEPAd Protein by the Transformed GAP3 Strain of A. niger

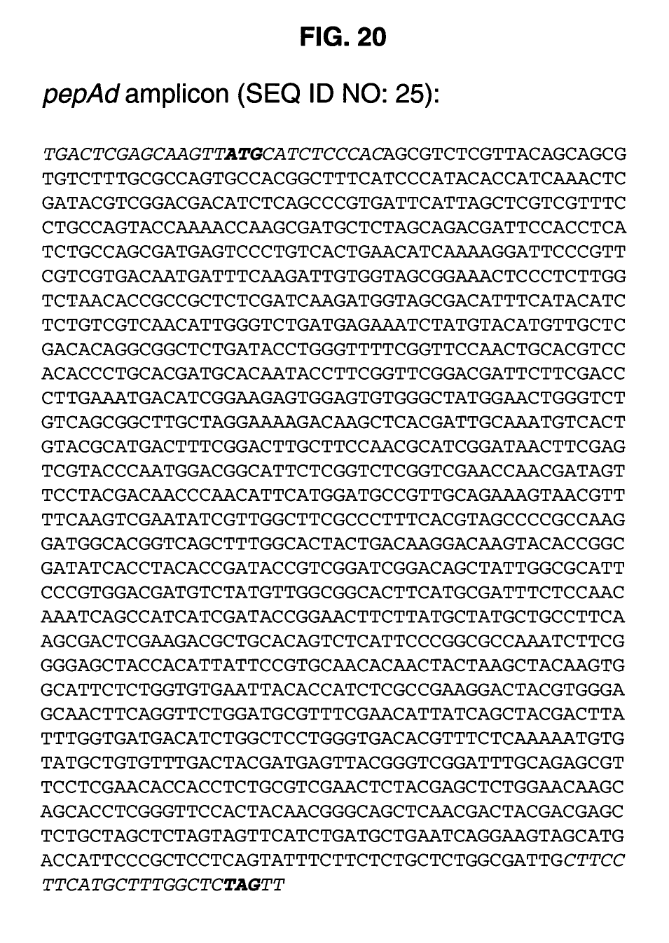

A recombinant expression plasmid for the full length A. niger pepAd gene was constructed as described for pepAa gene in Example 1 except that the two primers used in the Pfu PCR reaction containing genomic DNA template obtained from UVK143 strain were: TGACTCGAGCAAGTTATGCATCTCCCAC (SEQ ID NO: 18) and AACTAGAGCCAAAGCATGAAGGAAG (SEQ ID NO: 24) were used in a Pfu PCR reaction containing genomic DNA template obtained from UVK143 strain. The nucleotide sequence of the resulting PCR amplicon (SEQ ID NO: 25) is shown in FIG. 20 and encodes the full length PEPAd protein (SEQ ID NO: 4).

As in Example 1, the PCR amplicon was digested with restriction enzyme XhoI and cloned into XhoI and SnaBI digested pGAMD vector. The resulting plasmid vector, pGAMD-pepAd was confirmed by DNA sequencing to have a recombinant gene comprising pepAd inserted between the A. niger glucoamylase promoter and an A. tubingensis glucoamylase terminator.

The A. niger GAP3 strain was transformed (as described in Example 1) with the pGAMD-pepAd plasmid vector. Six transformants were selected and cultured in 30 ml Promosoy special broth (pH 6.2) for 6 days at 28° C. Culture broths were filtered and SDS-PAGE analysis of supernatants was used to detect presence of PEPAd protein.

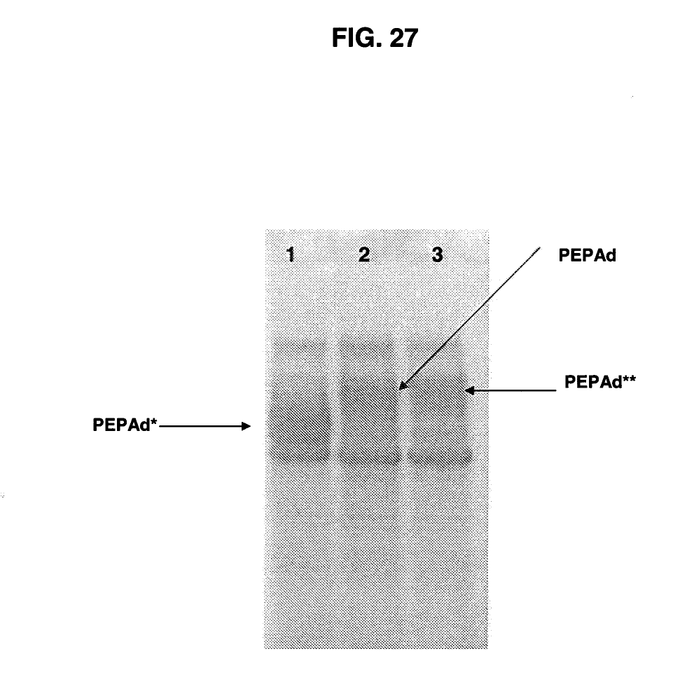

No protein of the expected MW of PEPAd was visualized in the gel. This result further confirms the conclusion of Example 5, that the truncated serine-rich C-terminal amino acid sequence (SEQ ID NO: 22) is a GPI anchor sequence that prevents secretion of the full length PEPAd protein (SEQ ID NO: 4) into the culture medium. Notably, upon screening additional transformants, two transformants were obtained that secreted full length PEPAd at a low level (see lane 2 in FIG. 27 for SDS gel analysis of strain #5). This “secreted” full length protein may result from the over-expression of PEPAd protein and subsequent leaking out through the cell wall, despite the presence of the GPI anchor sequence.

### Example 7

### Construction of a Recombinant Mutant pepAd Gene (pepAd**) and Production of the PEPAd** Protein by the Transformed GAP3 Strain of A. niger

A recombinant expression plasmid was constructed for a mutant of the A. niger pepAd gene (referred to herein as “pepAd”) in which a single glycine residue (G456) was deleted in the GPI anchor region. This mutation should eliminate anchoring of the expressed “PEPAd**” protein to the cell wall thereby allowing it to be secreted.

The mutant plasmid was constructed as described for pepAa in Example 1 except that four primers used in a Pfu PCR reaction containing genomic DNA template obtained from UVK143 strain. Two of the primers were: TGACTCGAGCAAGTTATGCATCTCCCAC (SEQ ID NO: 18) and ATGCTACTTGATTCAGCATCAGATGAAC (SEQ ID NO: 26). These were used to amplify DNA fragment encoded for the n-terminus of the protein. The other two primers were: TGCTGAATCAAGTAGCATGACCATTCC (SEQ ID NO: 29) and TAACTAGAGCCAAAGCATGAAGGAA (SEQ ID NO: 30). These were used to amplify DNA fragment encoded for the C-terminus of the protein.

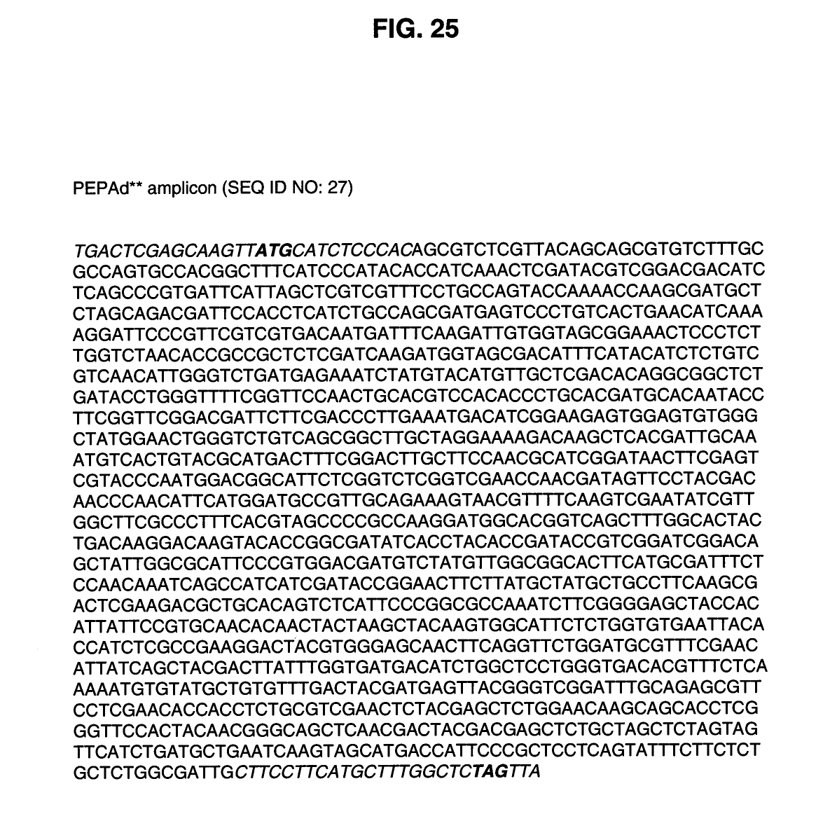

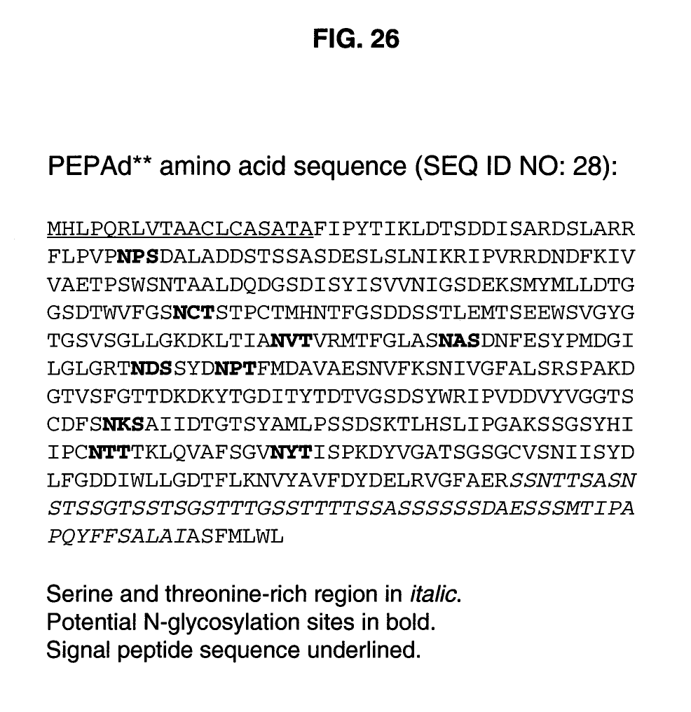

Fusion PCR was used to amplify the pepAd** gene fragment with the primers TGACTCGAGCAAGTTATGCATCTCCCAC (SEQ ID NO: 18) and TAACTAGAGCCAAAGCATGAAGGAA (SEQ ID NO: 30) and the N and C-terminal PCR amplicon as the DNA template. The nucleotide sequence of the resulting PCR amplicon (SEQ ID NO: 27) is shown in FIG. 25 and encodes the PEPAd** protein (SEQ ID NO: 28).

As in Example 1, the PCR amplicon was digested with restriction enzyme XhoI and cloned into XhoI and SnaBI digested pGAMD vector. The resulting plasmid vector, pGAMD-pepAd** was confirmed by DNA sequencing to have a recombinant gene comprising pepAd** inserted between the A. niger glucoamylase promoter and an A. tubingensis glucoamylase terminator.

The A. niger GAP3 strain was transformed (as described in Example 1) with the pGAMD-pepAd** plasmid vector. Twelve transformants were selected and cultured in 30 ml Promosoy special broth (pH 6.2) for 6 days at 28° C. Culture broths were filtered and SDS-PAGE analysis of supernatants was used to detect presence of PEPAd** protein. Two transformants obtained produced PEPAd** at low levels (see lane 3 of FIG. 27 for SDS gel analysis of strain #3-4). These results suggested that deletion of one amino acid at the GPI anchor site did not abolish the function of GPI anchor. However, as before, over-expression of the PEPAd** protein may lead to leakage out of cell wall, which is responsible for the observed low levels of “secreted” protein.

In a related experiment two adjacent amino acids in the GPI binding site (S455 and G456) were deleted. Again, the deletion did not completely abolish the normal function of the GPI anchor (not shown).

### Example 8

### Casein Proteolysis Activity Assay of Recombinant pepAa, pepAb, pepAc and Truncated pepAd

The four spore purified strains that produced the largest amount of their respective protein were selected for activity assay: strain #2-9 (PEPAa), strain #1-3 (PEPAb), strain #12-2 (PEPAc), and strain #9-2 (PEPAd*) (see Examples 1, 3, 4 and 5). Activity assays were also carried out using the corresponding parent strain (GAP3) and A. niger wild-type strain 13528 (CGMCC No. AS3.10145) as controls. All strains were cultured in 30 ml promosoy special broth for 6 days at 28° C. and supernatants from all flasks were used for proteases activity assay.

The casein activity assay was carried out as follows:

A casein substrate solution (0.5% w/v casein) was prepared in pH 3.0 Na₂HPO₄-citric acid buffer (4.11 ml 0.2 M Na₂HPO₄ and 15.89 ml 0.1 M citric acid per 20 ml).

After 10 min preincubation at 37° C., a PEPAx sample aliquot of 0.1 ml culture filtrate was added to the mixture of 0.9 ml H₂O and 2 ml casein solution and incubated at 37° C. for 20 min. The casein proteolysis reaction was quenched by addition of 3 ml 10% TCA.

A “blank” control was also assayed. The blank was prepared by first adding 3 ml 10% TCA to the mixture of 0.1 ml culture filtrate sample and 0.9 ml H₂O before adding 2 ml casein solution.

After TCA quenching of the casein proteolysis, samples and blanks were incubated at 37° C. for 30 min then centrifuged at 12000 rpm at RT for 2 min.

After centrifugation, 0.5 ml supernatant was mixed with 0.5 ml H₂O, added to 5 ml Folin-phenol reagent A (4% Na₂CO₄: 0.2 M NaOH: 1% CuSO₄: 2% potassium sodium tartrate, 50:50:1:1) and cultured at RT for 10 min. After addition of 0.5 ml Folin-phenol reagent B (1 N Folin phenol), the above solution was incubated at RT for 30 min.

The OD₆₆₀ of the quenched sample and blank after the folin-phenol treatment was determined using a 725C spectrophotometer. The blank OD₆₆₀ was subtracted from the sample OD₆₆₀ to determine the final value of OD₆₆₀ increase used to determine the amount of casein proteolysis.

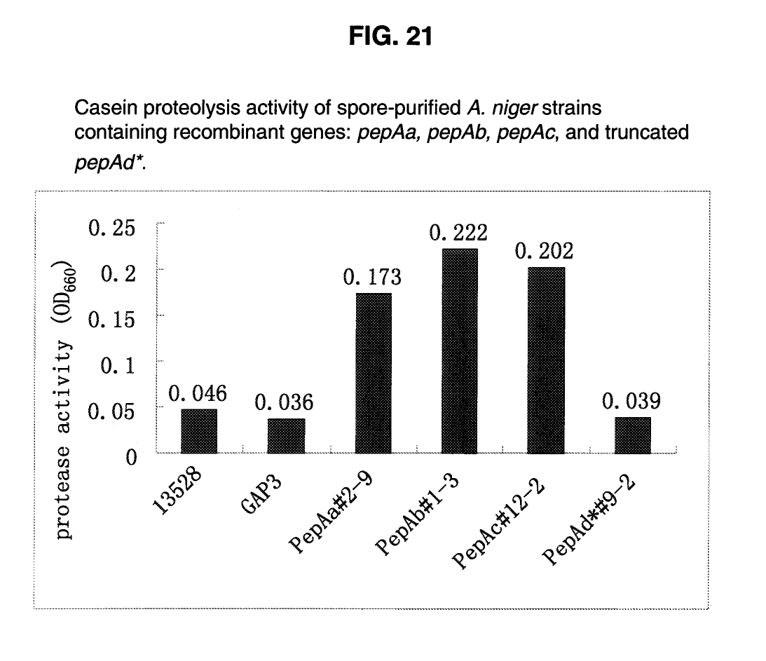

The casein proteolysis activities of the recombinant PEPAa, PEPAb, PEPAc and truncated PEPAd (PEPAd*), and the GAP3 and 13528 controls, are depicted in the chart shown in FIG. 21.

Relative to the assay activity of GAP3 (0.036) supernatant, the supernatant of strains recombinantly producing PEPAa, PEPAb, and PEPAc enzymes exhibited relative casein proteolysis activities increased 4.8-fold (0.173), 6.2-fold (0.222), and 5.6-fold (0.202), respectively.

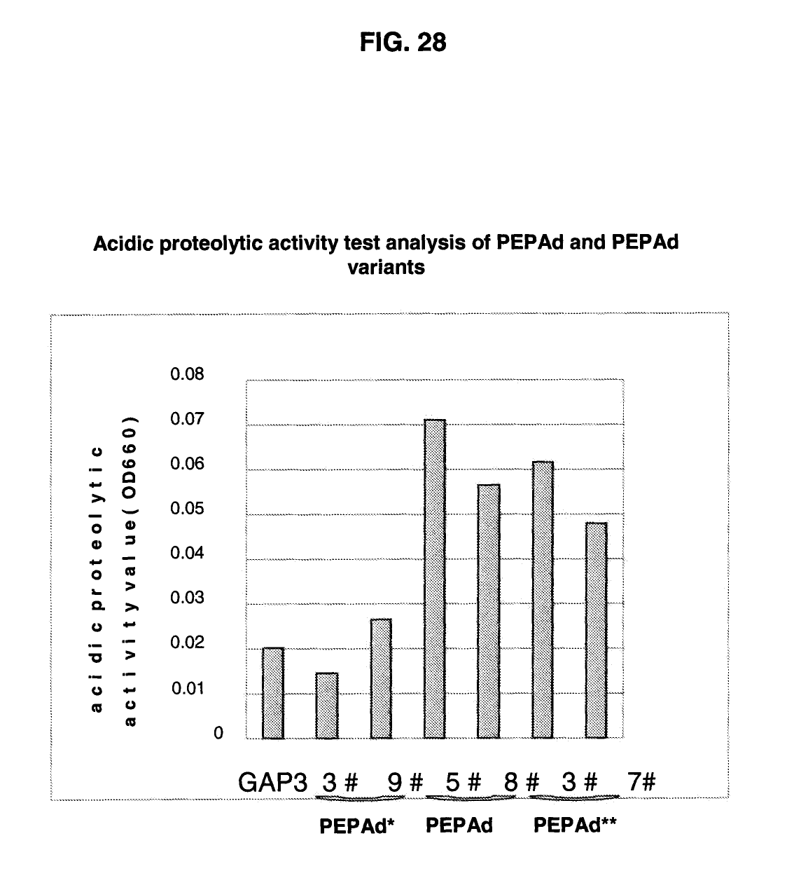

The casein proteolysis activities of the recombinant PEPAd was compared to GAP3 produced by the control strain. The casein proteolysis activities of the supernatants from strains #5 and #8 recombinant producing full length of PEPAd protein were 2.8 to 3.5 times of GAP3 supernatant, depicted in the chart shown in FIG. 28.

Despite SDS-PAGE showing that it overproduces the PEPAd* enzyme, the casein proteolysis activity of the supernatant from the strain #9-2 recombinantly producing PEPAd* was about the same as the GAP3 supernatant. The relatively low (or lacking) activity of the PEPAd* protein is possibly due to the truncation of the C-terminal GPI anchor sequence or result of proteolytic degradation by unknown proteases. Other mutations in the C-terminal GPI anchor sequence of PEPAd may result in a truncated PEPAd protein that is secreted in comparable amounts but has significant casein proteolysis activity.

As described, above, PEPAd** is a mutant variant of PEPAd wherein the amino acid glycine in the GPI anchor region was deleted. The casein proteolysis activities of recombinant PEPAd** was compared to that of PEPAd, PEPAd* and GAP3. The results are depicted in the chart shown in FIG. 28. The casein proteolysis activities of the supernatants from strain #3 and #7 recombinant producing PEPAd** protein were 2.3 to 3 times of GAP3 supernatant.

### Example 9

### Determination of pH for Optimal Activity of the Recombinantly Produced Proteins: PEPAa, PEPAb, PEPAc and PEPAd*

Protease activities of the four spore purified strains were determined as in Example 8, except that the casein solution was buffered at one of the following pH values: 2.0, 3.0, 4.0, 5.0, 6.0, and 7.0. The buffers used were as follows:

pH 2.0 buffer solution contained: 1.06 ml of 0.2 M Na₂HPO₄ and 18.94 ml of 0.1 M citric acid per 20 ml.

pH 3.0 buffer solution contained: 4.11 ml of 0.2 M Na₂HPO₄ and 15.89 ml of 0.1 M citric acid per 20 ml.

pH 4.0 buffer solution contained: 7.71 ml of 0.2 M Na₂HPO₄ and 12.29 ml of 0.1 M citric acid per 20 ml.

pH 5.0 buffer solution contained: 10.3 ml of 0.2 M Na₂HPO₄ and 9.7 ml of 0.1 M citric acid per 20 ml.

pH 6.0 buffer solution contained: 12.63 ml of 0.2 M Na₂HPO₄ and 7.37 ml of 0.1 M citric acid per 20 ml.

pH 7.0 buffer solution contained: 16.47 ml of 0.2 M Na₂HPO₄ and 3.53 ml of 0.1 M citric acid per 20 ml.

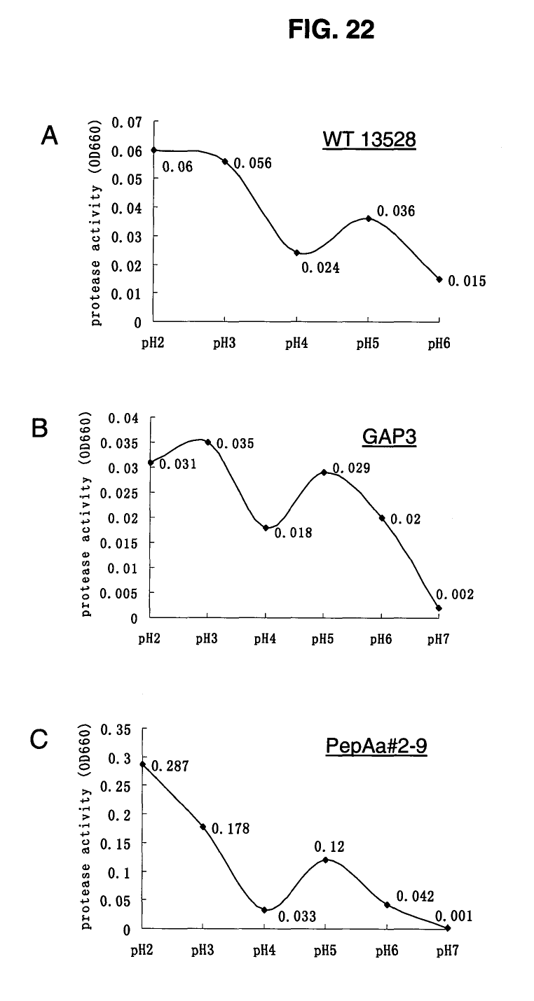

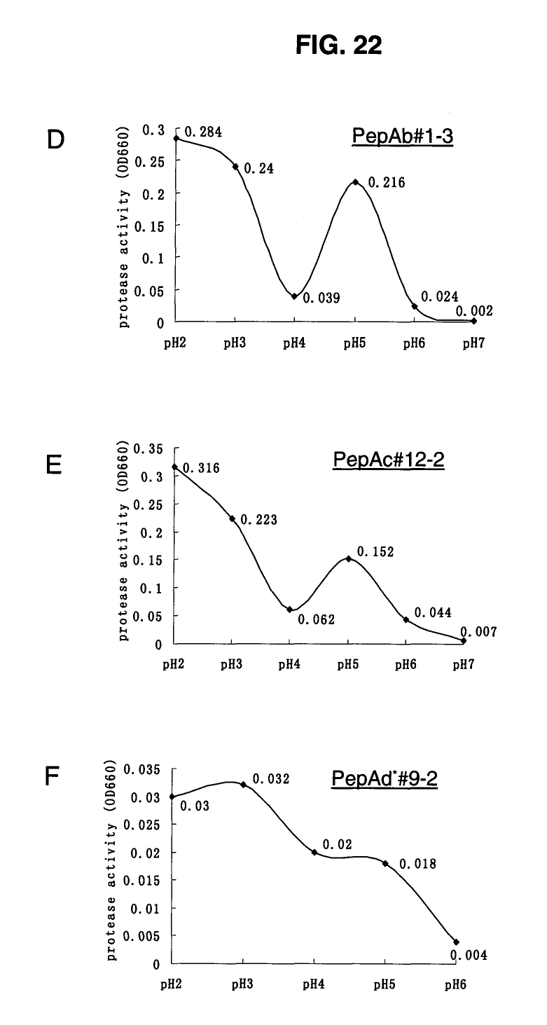

The resulting protease activity versus buffer solution pH for each of the four recombinantly produced protein supernatants was plotted in FIGS. 22 A-F. The pH profiles of the four recombinantly overproduced PEPAx proteins exhibit activity peaks at ˜pH 5 and ˜pH 3 (appears as a shoulder profile in PEPAa, Ab, and Ac). The similarity of these pH profiles indicates that the three recombinant enzymes share the same activity and mechanism.

### Example 10

### Determination of Temperature of Optimal Activity of the Recombinant Proteins: pepAa, pepAb, pepAc and Truncated pepAd*

Protease activities of the four spore-purified strains were determined as in Example 7, except that the PEPAx sample aliquot was incubated with the casein substrate solution for 20 minutes at each of the following temperatures: 28° C., 37° C., 50° C., 60° C., and 70° C.

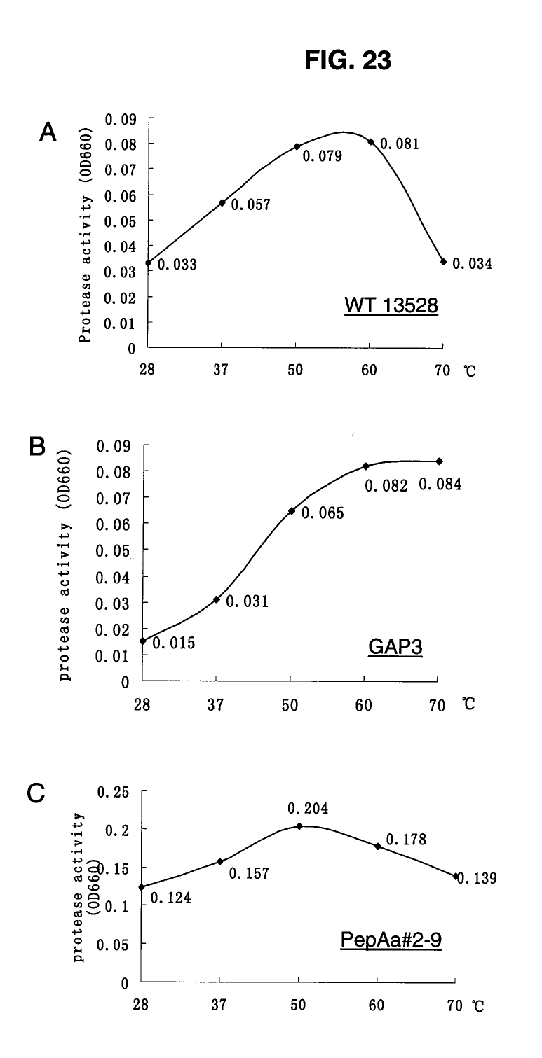

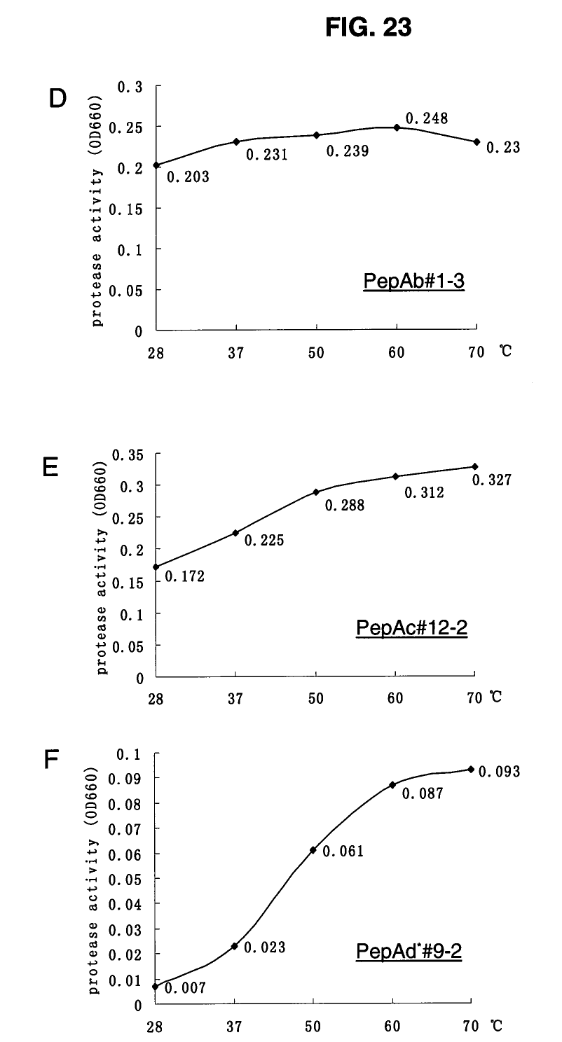

The resulting protease activity versus temperature for each of the four recombinantly produced protein supernatants was plotted in FIGS. 23 A-F.

The PEPAa activity-temperature profile shows a peak at about 50° C. and drops at higher temperature. PEPAb activity is relatively insensitive to temperature between about 37° C. and about 70° C. PEPAc activity increased continuously, nearly two-folds, between about 28° C. and 70° C. PEPAd* activity did not have any activity above the background and it is very similar to that of the GAP3 strain.

Those of skill in the art readily appreciate that the present compositions and methods are well adapted to carry out the objects and obtain the ends and advantages mentioned, as well as those inherent therein. The compositions and methods described herein are representative, exemplary embodiments, and are not intended as limitations on the scope of the compositions and methods.

While particular embodiments of the present compositions and methods have been illustrated and described, it will be apparent to those skilled in the art that various other changes and modifications can be made without departing from the spirit and scope of the compositions and methods. It is therefore intended to cover in the appended claims all such changes and modifications that are within the scope of the compositions and methods.

The compositions and methods illustratively described herein suitably may be practiced in the absence of any element(s) or limitation(s) which is not specifically disclosed herein. The terms and expressions which have been employed are used as terms of description and not of limitation, and there is no intention that in the use of such terms and expressions of excluding any equivalents of the features shown and described or portions thereof.

The compositions and methods have been described broadly and generically herein. Each of the narrower species and subgeneric groupings falling within the generic disclosure also form part of the compositions and methods. This includes the generic description of the compositions and methods with a proviso or negative limitation removing any subject matter from the genus, regardless of whether or not the excised material is specifically recited herein.

All patents and publications are herein incorporated by reference to the same extent as if each individual publication was specifically and individually indicated to be incorporated by reference.

What is claimed is:

1. A filamentous fungal cell comprising an inactivated pepA gene and a recombinant gene comprising a pepA homolog selected from the group consisting of pepAa, pepAb, pepAc, and pepAd.

2. The filamentous fungal cell of claim 1, wherein the recombinant gene comprises a promoter and a terminator.

3. The filamentous fungal cell of claim 1, wherein the recombinant gene comprises an A. niger glucoamylase promoter and an A. tubingensis glucoamylase terminator.

4. The filamentous fungal cell of claim 1, wherein the recombinant gene comprises a nucleotide sequence selected from SEQ ID NOs: 7, 12, 17, and 20.

5. The filamentous fungal cell of claim 1, wherein said filamentous fungus is selected from the group consisting of an Aspergillus spp., a Rhizopus spp., a Trichoderma spp., and a Mucor spp.

6. The filamentous fungal cell of claim 4, wherein said filamentous fungus is an Aspergillus spp. selected from the group consisting of A. oryzae, A. niger, A. awamori, A. nidulans, A. sojae, A. japonicus, A. kawachi, and A. aculeatus.

7. The filamentous fungal cell of claim 5, wherein the filamentous fungus is A. niger.

8. The filamentous fungal cell of claim 1, wherein the pepA homolog encodes a polypeptide secreted by the cell.

9. The filamentous fungal cell of claim 1, wherein the pepA homolog encodes a polypeptide having at least 85% identity to an amino acid sequence selected from the group consisting of SEQ ID NOs: 1, 2, 3, 4, 21, 23, and 28.

10. The filamentous fungal cell of claim 1, wherein the cell secretes the polypeptide encoded by the pepA homolog in an amount at least about 10% to about 1000% greater than a corresponding parent strain.

11. The filamentous fungal cell of claim 1, wherein the polypeptide encoded by the pepA homolog is at least about 10% of the total protein produced by the cell.

12. The filamentous fungal cell of claim 1, wherein the secreted protease activity of the cell is at least about 0.5-fold to about 100-fold greater than the secreted protease activity of a corresponding parent strain.

13. An isolated nucleic acid comprising an A. niger glucoamylase promoter sequence, a pepA homolog sequence, and an A. tubingensis glucoamylase terminator sequence, wherein the pepA homolog sequence encodes a polypeptide having at least 85% identity to an amino acid sequence selected from the group consisting of SEQ ID NOs: 1, 2, 3, 4, 21, 23, and 28.

14. The isolated nucleic acid of claim 13, wherein the pepA homolog comprises a nucleotide sequence selected from the group consisting of SEQ ID NOs: 7, 9, 12, 17, 20, 25, and 27.

15. A vector comprising an A. niger glucoamylase promoter sequence, a pepA homolog sequence, and an A. tubingensis glucoamylase terminator sequence, wherein the pepA homolog sequence encodes a polypeptide having at least 85% identity to an amino acid sequence selected from the group consisting of SEQ ID NOs: 1, 2, 3, 4, 21, 23, and 28.

16. The vector of claim 15, wherein the pepA homolog sequence comprises a nucleotide sequence selected from the group consisting of SEQ ID NOs: 7, 9, 12, 17, 20, 25, and 27.

17. The vector of claim 15, wherein the vector is a plasmid.

18. The vector of claim 15, wherein the plasmid comprises the pGAMD with a pepA homolog sequence insert.

19. The vector of claim 15, wherein the plasmid is selected from the group consisting of pGAMD-pepAa, pGAMD-pepAb, pGAMD-pepAc, pGAMD-pepAd, pGAMD-pepAd*, pGAMD-pepAa*, and pGAMD-pepAd*.

20. A method for producing an acidic protease comprising:
a) introducing a nucleic acid into a filamentous fungal cell, wherein the cell comprises an inactivated pepA gene and wherein the nucleic acid comprises a promoter sequence, a pepA homolog sequence, and a terminator sequence; and
b) growing the cell under conditions suitable for producing the acidic protease.

21. The method according to claim 20, wherein the promoter comprises an A. niger glucoamylase promoter sequence and the terminator comprises an A. tubingensis glucoamylase terminator sequence.

22. The method according to claim 20, wherein the pepA homolog sequence encodes a polypeptide having at least 85% identity to an amino acid sequence selected from the group consisting of SEQ ID NOs: 1, 2, 3, 4, 21, 23, and 28.

23. The method according to claim 20, wherein the pepA homolog sequence comprises a nucleotide sequence selected from the group consisting of SEQ ID NOs: 7, 9, 12, 17, 20, 25, and 27.

24. The method according to claim 20, wherein introducing the nucleic acid into the filamentous fungal cell comprises transforming the cell with a vector.

25. The method according to claim 20, wherein the vector is a plasmid.

26. The method according to claim 20, wherein the plasmid comprises the pGAMD with a pepA homolog sequence insert.

27. The method according to claim 20, wherein the plasmid is selected from the group consisting of pGAMD-pepAa, pGAMD-pepAb, pGAMD-pepAc, pGAMD-pepAd, pGAMD-pepAd*, pGAMD-pepAa*, and pGAMD-pepAd*.

28. The method according to claim 20, wherein the method further comprises recovering the protein.

29. An isolated nucleic acid comprising a sequence of SEQ ID NO: 20.

30. An isolated nucleic acid comprising a sequence encoding a polypeptide having at least 85% amino acid sequence identity to SEQ ID NO: 21.

31. An isolated polypeptide having at least 85% amino acid sequence identity to SEQ ID NO: 21.

32. An enzyme composition comprising a polypeptide having at least 85% amino acid sequence identity to SEQ ID NO: 21.

33. A vector comprising a nucleotide sequence of SEQ ID NO: 20.

34. A vector comprising a nucleotide sequence encoding a polypeptide having at least 85% amino acid sequence identity to SEQ ID NO: 21.
